(19) United States
(12) Patent Application Publication
(10) Pub. No.: US 2020/0157088 A1
(43) Pub. Date: May 21, 2020

(54) BENZENE FUSED HETEROCYCLIC COMPOUND AND USE THEREOF

(71) Applicant: TAIWANJ PHARMACEUTICALS CO., LTD., Zhubei City, Hsinchu County (TW)

(72) Inventors: Syaulan S. YANG, Zhubei City (TW); Kuang Yuan LEE, Hsinchu City (TW); Yan-Feng JIANG, Kaohsiung City (TW); Yu-Shiou FAN, Taipei City (TW); Meng Hsien LIU, Toufen City (TW); Sheng Hung LIU, Zhubei City (TW); Jhih-Liang HUANG, Puli (TW)

(73) Assignee: TAIWANJ PHARMACEUTICALS CO., LTD., Zhubei City, Hsinchu County (TW)

(21) Appl. No.: 16/620,702

(22) PCT Filed: Nov. 30, 2018
(86) PCT No.: PCT/US2018/063324

§ 371 Date: Dec. 9, 2019

Related U.S. Application Data
(60) Provisional application No. 62/593,411, filed on Dec. 1, 2017.

(51) Int. Cl. C07D 417/06; C07D 401/06; C07D 413/06; C07D 211/70; C07D 403/06; C07D 417/14; C07D 405/14; C07D 405/10

(52) U.S. Cl. CPC C07D 417/06; C07D 401/06; C07D 413/06; C07D 405/10; C07D 403/06; C07D 417/14; C07D 405/14; C07D 211/70

(57) ABSTRACT

The present disclosure provides a benzene fused heterocyclic compound of Formula (I): wherein (A) is a single or double bond; n is 0 or 1; X is —CH₂—, O, NR₁, or S; A is —C(Rₐ₁)(Rₐ₂)(Rₐ₃) or —N(Rₐ₁)(Rₐ₂), wherein Rₐ₁, Rₐ₂ and Rₐ₃ are independently selected from a group consisting of: H, alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂ RₐₐR_{bb} and —SO₂ NRₐₐR_{bb} which are optionally substituted by at least one substituent independently selected from a group consisting of: alkyl, cycloalkyl, heterocyclic alkyl, aryl, —Y_{bb}, —Ar_{bb}Y_{bb}, —OR_{cc}, and —OAr_{bb}Y_{bb}, wherein Rₐₐ, R_{bb} and R_{cc} independently are nil, H, halogen, alkyl, or aryl, Y_{bb} is CN or halogen, and Arₐₐ and Ar_{bb} independently are aryl or heteroaryl; R₁ is H or alkyl; R₂ is alkyl, cycloalkyl, heterocylic alkyl, aryl, heteroaryl, C₁-C₆ hydrocarbon optionally substituted by at least one substituent independently selected from a group consisting of: —R₂ₐOR_{2b}, —R₂ₐC(O)OR_{2b}R_{2c}, —R₂ₐC(O)R_{2b}R_{2c}, —R₂ₐC(O)NR_{2b}R_{2c}, —R₂ₐNR_{2b}C(O)NR_{2c}R_{2d}, —R₂ₐNR_{2b}C(O)R_{2c}R_{2d}, —R₂ₐNR_{2b}C(O)OR_{2c}R_{2d}, —R₂ₐSO₂R_{2b}R_{2c}, —R₂ₐNR_{2b}SO₂NR_{2c}R_{2d} and —R₂ₐSO₂NR_{2b}R_{2c}, optionally substituted by at least one substituent independently selected from a group consisting of alkyl, cycloalkyl, heterocyclic alkyl, heteroaryl, and aryl, wherein R₂ₐ, R_{2b}, R_{2c} and R_{2d} are independently selected from nil, H, halogen, alkyl, cycloalkyl, heterocyclic alkyl, heteroaryl, aryl or C₁-C₆ hydrocarbons, optionally substituted by at least one substituent independently selected from a group consisting of —OR₂ₑ, ═O, ═S, —SO₂R₂ₑ, —SO₂NR₂ₑR_{2f}, —NR_{2g}SO₂NR₂ₑR_{2f}, —NR_{2g}C(O)NR₂ₑR_{2f}, —C(O)NHR₂ₑ, —NHC(O)R₂ₑ, —NHC(O)OR₂ₑ, —NO₂, —CO₂R₂ₑ and —C(O)R₂ₑ, wherein R₂ₑ, R_{2f}, and R_{2g} independently are H or alkyl.

[symbol]

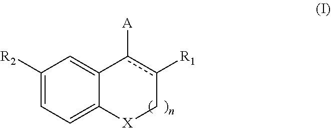

### TECHNICAL FIELD

The technical field relates to benzene fused heterocyclic compounds and their uses, and in particular to the pharmaceutical compositions comprising the same and their use as autotaxin inhibitors.

### BACKGROUND

Autotaxin (ATX) is a secreted enzyme of about 120 kDa in humans and is encoded by the ENPP2 gene. Autotaxin is also known as ectonucleotide pyrophosphatase/phosphodiesterase family member 2 (NPP2 or ENPP2) or lysophospholipase D.

Lysophosphatidic acid (LPA) activates at least six G-protein coupled receptors, which promote cell proliferation, survival, migration and muscle contraction while autotaxin has lysophospholipase D activity that converts lysophosphatidylcholine into lysophosphatidic acid. Autotaxin is important in generating the lipid signaling molecule LPA.

Non-alcoholic fatty liver disease (NAFLD) is the buildup of extra fat in liver cells that is not caused by alcohol. Non-alcoholic steatohepatitis (NASH) is the most extreme form of NAFLD. Moreover, NASH is regarded as a major cause of cirrhosis of the liver of unknown cause, and ATX-LPA signaling has been implicated in hepatic fibrogenesis.

Idiopathic pulmonary fibrosis (IPF) is a chronic, relentlessly progressive fibrotic disorder of the lungs occurring mainly in older adults. It is reported that in both murine and human fibrotic lungs, increased concentrations of ATX can be detected.

Autotaxin and LPA are also involved in numerous inflammatory-driven diseases such as asthma and arthritis. In addition, Autotaxin and LPA have been shown to be involved in many cancers.

Accordingly, development of autotaxin inhibitors for treating diseases such as cancer, NAFLD, IPF, etc. is needed.

### SUMMARY

According to some embodiments, the present disclosure provides a benzene fused heterocyclic compound of Formula (I), or a pharmaceutical acceptable salt, solvate, hydrate, geometric isomer, enantiomer, diastereoisomer or racemate thereof:

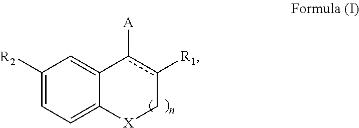

wherein [symbol] is a single or double bond; n is 0 or 1; X is —CH₂—, O, NR₁, or S; A is —C(Rₐ₁)(Rₐ₂)(Rₐ₃) or —N(Rₐ₁)(Rₐ₂), wherein Rₐ₁, Rₐ₂ and Rₐ₃ are independently selected from a group consisting of: H, alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂ RₐₐR_{bb} and —SO₂ NRₐₐR_{bb} which are optionally substituted by at least one substituent independently selected from a group consisting of: alkyl, cycloalkyl, heterocyclic alkyl, aryl, —Y_{bb}, —Ar_{bb}Y_{bb}, —OR_{cc}, and —OAr_{bb}Y_{bb}, wherein Rₐₐ, R_{bb} and R_{cc} independently are nil, H, halogen, alkyl, or aryl, Y_{bb} is CN or halogen, and Arₐₐ and Ar_{bb} independently are aryl or heteroaryl; R₁ is H or alkyl; R₂ is alkyl, cycloalkyl, heterocylic alkyl, aryl, heteroaryl, C₁-C₆ hydrocarbon optionally substituted by at least one substituent independently selected from a group consisting of: —R₂ₐOR_{2b}, —R₂ₐC(O)OR_{2b}R_{2c}, —R₂ₐC(O)R_{2b}R_{2c}, —R₂ₐC(O)NR_{2b}R_{2c}, —R₂ₐNR_{2b}C(O)NR_{2c}R_{2d}, —R₂ₐNR_{2b}C(O)R_{2c}R_{2d}, —R₂ₐNR_{2b}C(O)OR_{2c}R_{2d}, —R₂ₐSO₂R_{2b}R_{2c}, —R₂ₐNR_{2b}SO₂NR_{2c}R_{2d} and —R₂ₐSO₂NR_{2b}R_{2c}, optionally substituted by at least one substituent independently selected from a group consisting of alkyl, cycloalkyl, heterocyclic alkyl, heteroaryl, and aryl, wherein R₂ₐ, R_{2b}, R_{2c} and R_{2d} are independently selected from nil, H, halogen, alkyl, cycloalkyl, heterocyclic alkyl, heteroaryl, aryl or C₁-C₆ hydrocarbons, optionally substituted by at least one substituent independently selected from a group consisting of —OR₂ₑ, ═O, ═S, —SO₂R₂ₑ, —SO₂NR₂ₑR_{2f}, —NR_{2g}SO₂NR₂ₑR_{2f}, —NR_{2g}C(O)NR₂ₑR_{2f}, —C(O)NHR₂ₑ, —NHC(O)R₂ₑ, —NHC(O)OR₂ₑ, —NO₂, —CO₂R₂ₑ and —C(O)R₂ₑ, wherein R₂ₑ, R_{2f}, and R_{2g} independently are H or alkyl.

According to other embodiments, the present disclosure also provides a pharmaceutical composition, comprising: a therapeutically effective amount of the benzene fused heterocyclic compound of the present disclosure; and a pharmaceutically acceptable carrier.

According to still other embodiments, the present disclosure further provides a method for inhibiting the activity of autotaxin in environment, comprising: contacting the environment an effective amount of the benzene fused heterocyclic compound of the present disclosure or the pharmaceutical composition of the present disclosure.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

Novel Compounds

The present disclosure provides a benzene fused heterocyclic compound of Formula (I), or a pharmaceutical acceptable salt, solvate, hydrate, enantiomer, or diastereoisomer thereof:

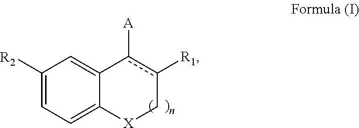

wherein [symbol] is a single or double bond; n is 0 or 1; X is —CH₂—, O, NR₁, or S; A is —C(Rₐ₁)(Rₐ₂)(Rₐ₃) or —N(Rₐ₁)(Rₐ₂), wherein Rₐ₁, Rₐ₂ and Rₐ₃ are independently selected from a group consisting of: H, alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂ RₐₐR_{bb} and —SO₂ NRₐₐR_{bb} which are optionally substituted by at least one substituent independently selected from a group consisting of: alkyl, cycloalkyl, heterocyclic alkyl, aryl, —Y_{bb}, —Ar_{bb}Y_{bb}, —OR_{cc}, and —OAr_{bb}Y_{bb}, wherein Rₐₐ, R_{bb} and R_{cc} independently are nil, H, halogen, alkyl, or aryl, Y_{bb} is CN or halogen, and Arₐₐ and Ar_{bb} independently are aryl or heteroaryl; R₁ is H or alkyl; R₂ is alkyl, cycloalkyl, heterocylic alkyl, aryl, heteroaryl, C₁-C₆ hydrocarbon optionally substituted by at least one substituent independently selected from a group consisting of: —R₂ₐOR_{2b}, —R₂ₐC(O)OR_{2b}R_{2c}, —R₂ₐC(O)R_{2b}R_{2c}, —R₂ₐC(O)NR_{2b}R_{2c}, —R₂ₐNR_{2b}C(O)NR_{2c}R_{2d}, —R₂ₐNR_{2b}C(O)R_{2c}R_{2d}, —R₂ₐNR_{2b}C(O)OR_{2c}R_{2d}, —R₂ₐSO₂R_{2b}R_{2c}, —R₂ₐNR_{2b}SO₂NR_{2c}R_{2d} and —R₂ₐSO₂NR_{2b}R_{2c}, optionally substituted by at least one substituent independently selected from a group consisting of alkyl, cycloalkyl, heterocyclic alkyl, heteroaryl, and aryl, wherein R₂ₐ, R_{2b}, R_{2c} and R_{2d} are independently selected from nil, H, halogen, alkyl, cycloalkyl, heterocyclic alkyl, heteroaryl, aryl or C₁-C₆ hydrocarbons, optionally substituted by at least one substituent independently selected from a group consisting of —OR₂ₑ, ═O, ═S, —SO₂R₂ₑ, —SO₂NR₂ₑR_{2f}, —NR_{2g}SO₂NR₂ₑR_{2f}, —NR_{2g}C(O)NR₂ₑR_{2f}, —C(O)NHR₂ₑ, —NHC(O)R₂ₑ, —NHC(O)OR₂ₑ, —NO₂, —CO₂R₂ₑ and —C(O)R₂ₑ, wherein R₂ₑ, R_{2f}, and R_{2g} independently are H or alkyl.

In some embodiments of the present disclosure, the benzene fused heterocyclic compound of Formula (I) can be Formula (II), or a pharmaceutical acceptable salt, solvate, hydrate, enantiomer, or diastereoisomer thereof:

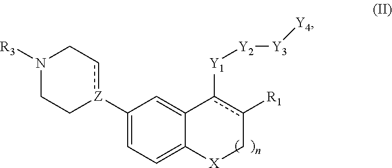

wherein - - - is a single or double bond;

n is 0 or 1;

X is —CH₂—, O, NR₁, or S;

Y₁ is —C(Rₐ₁)(Rₐ₂)— or —N(Rₐ₁)—, wherein Rₐ₁ and Rₐ₂ are independently selected from a group consisting of:

H, alkyl, cycloalkyl, heterocyclic alkyl, aryl, heteroaryl and C₁-C₃ hydrocarbons;

Y₂ is alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂RₐₐR_{bb} or —SO₂NRₐₐR_{bb}, wherein Rₐₐ and R_{bb} independently are nil, H, halogen, alkyl, or aryl;

Y₃ is nil, H, CN, halogen, alkyl, cycloalkyl, heterocyclic alkyl, aryl, heteroaryl or C₁-C₃ hydrocarbon, optionally substituted by at least one substituent independently selected from a group consisting of H, alkyl, and halogen;

Y₄ is nil, H, halogen, aryl or heteroaryl, optionally substituted by at least one substituent independently selected from a group consisting of H, alkyl, and halogen;

R₁ is H or alkyl;

Z is C or N;

R₃ is —R₃ₐOR_{3b}, —R₃ₐC(O)OR_{3b}R_{3c}, —R₃ₐC(O)R_{3b}R_{3c}, —R₃ₐC(O)NR_{3b}R_{3c}, —R₃ₐNR_{3b}C(O)NR_{3c}R_{3d}, —R₃ₐNR_{3b}C(O)R_{3c}R_{3d}, —R₃ₐNR_{3b}C(O)OR_{3c}R_{3d}, —R₃ₐSO₂ R_{3b}R_{3c}, —R₃ₐNR_{3b}SO₂NR_{3c}R_{3d}, or —R₃ₐSO₂NR_{3b}R_{3c}, optionally substituted by at least one substituent independently selected from a group consisting of alkyl, cycloalkyl, heterocyclic alkyl, heteroaryl, and aryl,

wherein R₃ₐ, R_{3b}, R_{3c} and R_{3d} are independently selected from a group consisting of nil, H, halogen, alkyl, cycloalkyl, heterocyclic alkyl, heteroaryl, aryl and C₁-C₆ hydrocarbon, optionally substituted by at least one substituent independently selected from a group consisting of —OR₃ₑ, ═O, ═S, —SO₂R₃ₑ, —SO₂NR₃ₑR_{3f}, —NR_{3g}SO₂NR₃ₑR_{3f}, —NR_{3g}C(O)NR₃ₑR_{3f}, —C(O)NHR₃ₑ, —NHC(O)R₃ₑ, —NHC(O)OR₃ₑ, —NO₂, —CO₂R₃ₑ and —C(O)R₃ₑ,

wherein R₃ₑ, R_{3f}, and R_{3g} independently are H or alkyl.

### Definitions of Terms

Unless otherwise indicated, the term “alkyl” means a straight chain, branched and/or cyclic hydrocarbon having from 1 to 10 (e.g., 1 to 10, 1 to 9, 1 to 8, 1 to 7, 1 to 6, 1 to 5, 1 to 4, 1 to 3, 1 to 2, or 1) carbon atoms. Alkyl moieties having from 1 to 4 carbons (C₁₋₄ alkyl) are referred to as “lower alkyl.” Examples of alkyl groups include methyl, ethyl, propyl, isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is substituted C₂₋₁₀ alkyl.

The term “cycloalkyl” refers to a saturated hydrocarbon mono- or multi-ring (e.g., fused, bridged, or spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, and adamantyl.

“Heterocyclic alkyl” refers to a radical of a 3- to 10-membered non-aromatic ring system having ring carbon atoms and 1 to 4 ring heteroatoms, wherein each heteroatom is independently selected from nitrogen, oxygen, sulfur, phosphorus, and silicon (“3-10 membered heterocyclic alkyl”). In heterocyclyl groups that contain one or more nitrogen atoms, the point of attachment can be a carbon or nitrogen atom, as valency permits. Unless otherwise specified, each instance of heterocyclic alkyl is independently optionally substituted, i.e., unsubstituted (an “unsubstituted heterocyclic alkyl”) or substituted (a “substituted heterocyclic alkyl”) with one or more substituents. In some embodiments, a heterocyclyl group is a 5-8 membered non-aromatic ring system having ring carbon atoms and 1-4 ring heteroatoms, wherein each heteroatom is independently selected from nitrogen, oxygen, and sulfur. In some embodiments, a heterocyclic alkyl group is a 5-6 membered non-aromatic ring system having ring carbon atoms and 1-4 ring heteroatoms, wherein each heteroatom is independently selected from nitrogen, oxygen, and sulfur. In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclic alkyl has one ring heteroatom selected from nitrogen, oxygen, and sulfur. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl.

Unless otherwise indicated, the term “aryl” means an aromatic ring or a partially aromatic ring system composed of carbon and hydrogen atoms. An aryl moiety may comprise multiple rings bound or fused together. Examples of aryl moieties include naphthyl, and phenyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is a substituted phenyl.

Unless otherwise indicated, the term “heteroaryl” means an aryl moiety wherein at least one of its carbon atoms has been replaced with a heteroatom (e.g., N, O or S). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl.

Unless otherwise indicated, the term “alkoxy” or “alkoxyl” means an —O— alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃. The term “lower alkoxy” refers to —O-(lower alkyl), such as —OCH₃ and —OCH₂CH₃.

Unless otherwise indicated, the terms “halogen” and “halo” encompass fluoro, chloro, bromo, and iodo.

The term “amino” refers to a moiety of the formula: —N(R)₂, wherein each instance of R is independently a substituent described herein, or two instances of R are connected to form substituted or unsubstituted heterocyclyl. In certain embodiments, the amino is unsubstituted amino (i.e., —NH₂). In certain embodiments, the amino is a substituted amino group, wherein at least one instance of R is not hydrogen.

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, —OH, —CHO, alkoxy, alkanoyloxy (e.g., —OAc), alkenyl, alkyl (e.g., methyl, ethyl, propyl, t-butyl), aryl, aryloxy, halo, or haloalkyl (e.g., —CCl₃, —CF₃, —C(CF₃)₃).

Unless otherwise indicated, one or more adjectives immediately preceding a series of nouns is to be construed as applying to each of the nouns. For example, the phrase “optionally substituted alky, cycloalkyl, heterocyclic alkyl, aryl, or heteroaryl” has the same meaning as “optionally substituted alky, optionally substituted cycloalkyl, optionally substituted heterocyclic alkyl, optionally substituted aryl, or optionally substituted heteroaryl.”

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

Unless otherwise indicated, “an effective amount” of a compound is an amount sufficient to provide a therapeutic or positive benefit in the treatment or management of a disease, environment or condition, or to delay or minimize one or more symptoms associated with the disease, environment or condition. An effective amount of a compound is an amount of therapeutic agent, alone or in combination with other therapies, which provides a therapeutic benefit in the treatment or management of the disease, environment or condition. The term “effective amount” can encompass an amount that improves overall therapy, reduces or avoids symptoms or causes of a disease, environment or condition, or enhances the therapeutic efficacy of another therapeutic agent.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4- salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “pharmaceutically acceptable carrier” refers to a carrier, whether diluent or excipient, that is compatible with the other ingredients of a formulation and not deleterious to the recipient thereof. A usable pharmaceutically acceptable carrier are disclosed in various references including Handbook of Pharmaceuticals Excipients edited by Raymond C Rowe, Paul J Sheskey, and Marian E Quinn. In a unlimited embodiment, said pharmaceutically acceptable carrier can be selected from the group consisting of inert diluents, dispersing and/or granulating agents, surface active agents and/or emulsifiers, disintegrating agents, binding agents, preservatives, buffering agents, lubricating agents, and/or oils. Said compositions optionally further comprise at least one of additional biologically active compounds or agents.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture” or “racemate”.

Pharmaceutical Formulation

In some embodiments, the benzene fused heterocyclic compounds of the present disclosure are useful as the pharmaceutical active agents. More preferably, the compounds of the present disclosure are formulated into pharmaceutical formulations for administration. In some embodiments, the compounds of the present disclosure may be formulated for administering to an environment (such as a cell). In some embodiments, the pharmaceutical composition comprises a therapeutically effective amount of a compound of Formula (I) or Formula (II) of the present disclosure.

In some embodiments, the compound of Formula (I) is present at a level of about 0.1% to 99% by weight, based on the total weight of the pharmaceutical composition. In some embodiments, the compound of Formula (I) is present at a level of at least 1% by weight, based on the total weight of the pharmaceutical composition. In certain embodiments, the compound of Formula (I) is present at a level of at least 5% by weight, based on the total weight of the pharmaceutical composition. In still other embodiments, the compound of Formula (I) is present at a level of at least 10% by weight, based on the total weight of the pharmaceutical composition. In still yet other embodiments, the compound of Formula (I) is present at a level of at least 25% by weight, based on the total weight of the pharmaceutical composition.

In general, the pharmaceutical composition of the present disclosure are prepared by uniformly and intimately admixing the compounds of the present disclosure with a liquid or finely divided solid carrier, or both, and then, if necessary, shaping the resulting mixture. In some embodiments, the pharmaceutically acceptable carrier is selected from the group consisting of inert diluents, dispersing and/or granulating agents, surface active agents and/or emulsifiers, disintegrating agents, binding agents, preservatives, buffering agents, lubricating agents, and oils.

In some embodiments, the present disclosure provides the pharmaceutical compositions comprising the compounds of Formula (I) or Formula (II) described herein, or pharmaceutically acceptable salts thereof, in pharmaceutically acceptable carriers for any suitable route of administration, including but not limited to, oral, intravenous, intramuscular, cutaneous, subcutaneous, intrathecal, intradermal, transdermal, implantation, sublingual, buccal, rectal, vaginal, ocular, otic, nasal, inhalation, topical, buccal, parenteral and nebulization administration.

Synthesis of Novel Compounds

The benzene fused heterocyclic compounds in the present disclosure can be prepared by any of the methods known in the art. For example, the following schemes illustrate the typical synthetic routes for preparing the benzene fused heterocyclic compounds in the present disclosure.

### EXAMPLES

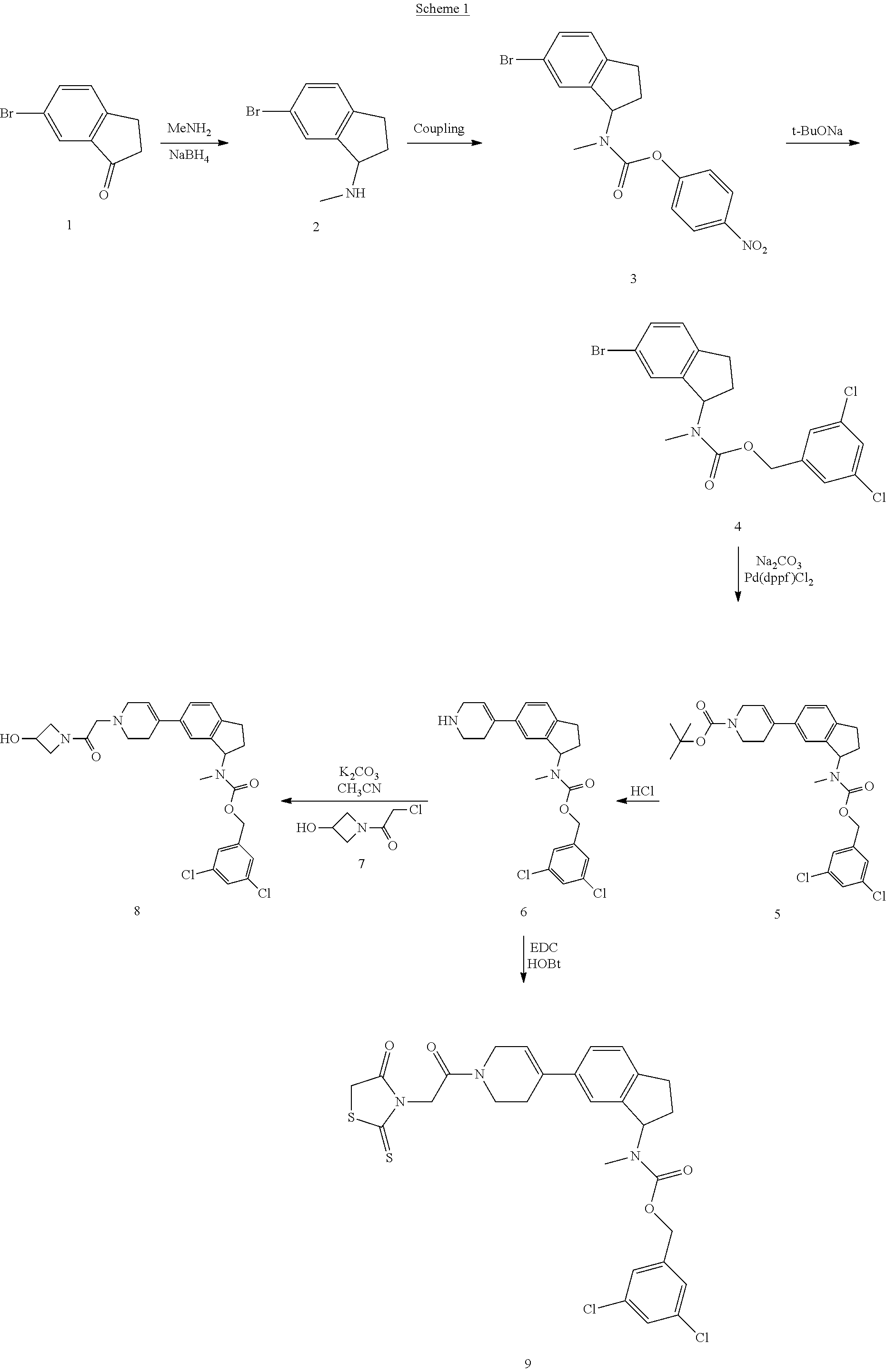

For the preparation of Compounds 8-9, please refer to Scheme 1 and the following details:

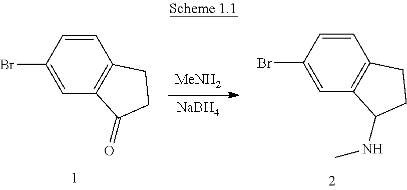

6-bromoindanole (4.3 g/20.3 mmole) (Compound 1) and methylamine (20 mL, 9.8 M in MeOH), in methanol (50 mL) were charged into a round bottom flask and stirred for about 3.5 hours at room temperature to form a solution. Sodium borohydride (1.2 g) was slowly added to the solution at room temperature to form a mixture, and then the mixture is stirred and maintained for completion of the reaction overnight. After that, the solvent and excess methylamine in the mixture was removed under vacuum to produce a residue. Ice-water was added to the residue and then a brown black solid was found, filtered, collected and washed by NaHCO₃ (aq). Next, the solid was dried under vacuum to afford a product (4.04 g, 87% yield). The product was Compound 2 (6-bromo-N-methyl-2,3-dihydro-1H-inden-1-amine). The product was used in the next step without further purification.

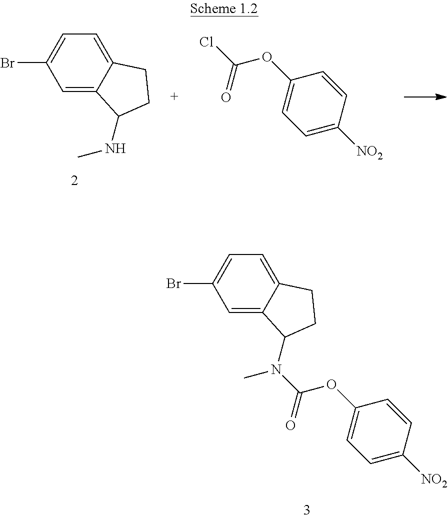 [SMILES: CNC1CCc2ccc(Br)cc21 ; O=C(Cl)Oc1ccc([N+](=O)[O-])cc1 ; CN(C(=O)Oc1ccc([N+](=O)[O-])cc1)C1CCc2ccc(Br)cc21]

TEA (1.88 mL, 2.0 eq.) and 4-nitrophenyl chloroformate (2.1 g, 1.5 eq.) were added to a mixture of Compound 2 (6-bromo-N-methyl-2,3-dihydro-1H-inden-1-amine) (1.526 g, 6.7487 mmol) and dry CH₂Cl₂ (10.0 mL) at 0° C. The resulting reaction mixture was stirred at room temperature overnight and the completion of reaction was confirmed by TLC. The resulting crude product was purified by column chromatography (EtOAc/Hexanes=1/4) to give a yellow oil. The yellow oil was Compound 3 (4-nitrophenyl (6-bromo-2,3-dihydro-1H-inden-1-yl)(methyl) carbamate) (817 mg, 31%).

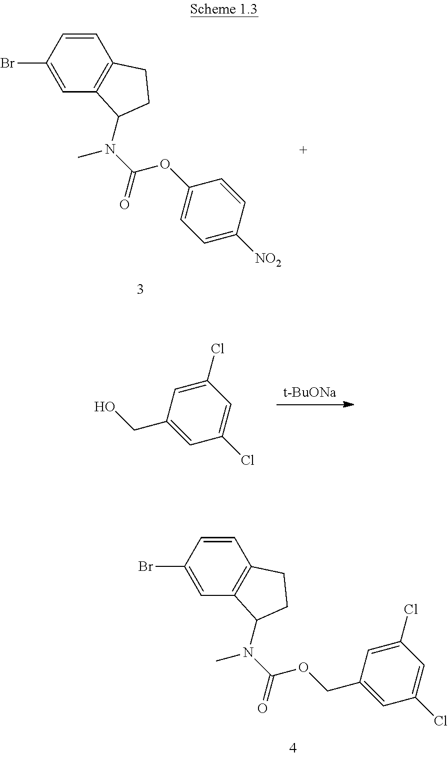 [SMILES: "t-BuONa" ; CN(C(=O)Oc1ccc([N+](=O)[O-])cc1)C1CCc2ccc(Br)cc21 ; OCc1cc(Cl)cc(Cl)c1 ; CN(C(=O)OCc1cc(Cl)cc(Cl)c1)C1CCc2ccc(Br)cc21]

3,5-dichlorobenzyl alcohol (2929.3 mg, 2.0 eq.) and sodium tert-butoxide (1558.3 mg, 2.0 eq.) was added to a mixture of Compound 3 (4-nitrophenyl (6-bromo-2,3-dihydro-1H-inden-1-yl)(methyl) carbamate) (3.172 g, 8.1079 mmol) and dry THF (20.0 mL) at 0° C. The resulting mixture was stirred at room temperature overnight. When the reaction was completed (note: the color of the mixture solution converted from yellow to orange), the resulting mixture was acidified with 2 N HCl_{(aq.)} (note: the color of the mixture solution converted from orange to white) and extracted with EtOAc. The organic phase was dried with Na₂SO₄ and concentrated under reduced pressure to give a crude mixture. The crude mixture was purified by column chromatography (EtOAc/Hexanes=1/5) to give yellow oil. The yellow oil was Compound 4 (3,5-dichlorobenzyl (6-bromo-2,3-dihydro-1H-inden-1-yl)(methyl)carbamate) (3.117 g, 89.6%).

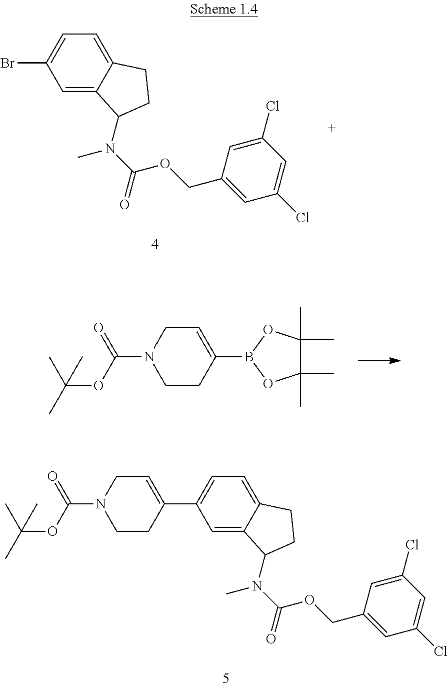

Na₂CO₃ (274 mg, 3.0 eq.), Pd(dppf)Cl₂ (31.5 mg, 0.05 eq.) and N-Boc-1,2,3,6-tetrahydropyridine-4-boronic acid pinacol ester (533 mg, 2.0 eq.) were added to a mixture of Compound 4 (3,5-dichlorobenzyl (6-bromo-2,3-dihydro-1H-inden-1-yl)(methyl) carbamate) (370 mg, 0.8622 mmol) and dry DMF (6.0 mL) at room temperature to form a reaction mixture. The reaction mixture was degassed and stirred under Ar_{(g)} for 15 minutes at room temperature. After that, the reaction mixture was stirred at 100° C. overnight. TLC was used to confirm the completion of reaction. After that, water was added to the reaction mixture and then the reaction mixture was extracted with EtOAc to obtain an organic phase. The organic phase was dried with Na₂SO₄ and concentrated under reduced pressure to give a crude mixture. The crude mixture was purified by column chromatography (EtOAc/Hexanes=1/8 to EtOAc/Hexanes=1/4) to give a green product. The green product was Compound 5, tert-butyl 4-(3-((((3,5-dichlorobenzyl)oxy)carbonyl)(methyl)amino)-2,3-dihydro-1H-inden-5-yl)-3,6-dihydropyridine-1(2H)-carboxylate (280 mg, 61%).

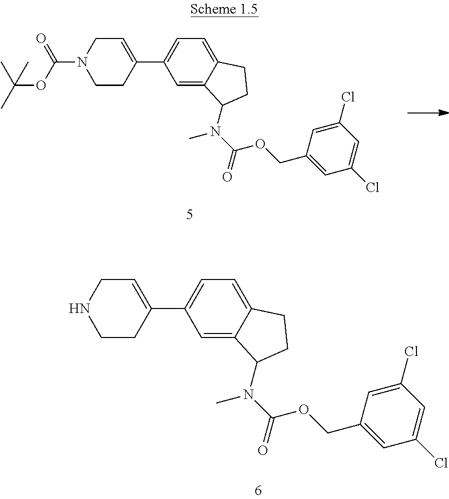 [SMILES: CN(C(=O)OCc1cc(Cl)cc(Cl)c1)C1CCc2ccc(C3=CCN(C(=O)OC(C)(C)C)CC3)cc21 ; CN(C(=O)OCc1cc(Cl)cc(Cl)c1)C1CCc2ccc(C3=CCNCC3)cc21]

4 N HCl (in 1,4-dioxane, 2.5 mL) was added to a mixture of Compound 5 (tert-butyl 4-(3-((((3,5-dichlorobenzyl)oxy)carbonyl)(methyl)amino)-2,3-dihydro-1H-inden-5-yl)-3,6-dihydropyridine-1(2H)-carboxylate) (280 mg, 0.5268 mmol) and dry CH₂Cl₂ (6.0 mL) at 0° C. to form a reaction mixture. The reaction mixture was stirred at room temperature overnight. TLC was used to confirm the completion of reaction. Saturated NaHCO₃ was added to the reaction mixture and then the reaction mixture was extracted with CH₂Cl₂ to obtain an organic phase. The organic phase was dried with Na₂SO₄ and concentrated under reduced pressure to give a brown product. The brown product was Compound 6, 3,5-dichlorobenzyl methyl(6-(1,2,3,6-tetrahydropyridin-4-yl)-2,3-dihydro-1H-inden-1-yl)carbamate (165 mg, 73%).

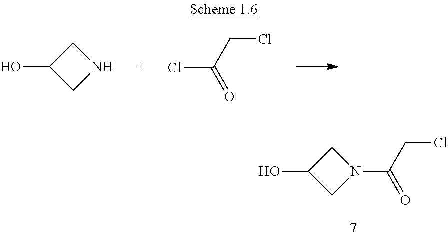 [SMILES: OC1CNC1 ; O=C(Cl)CCl ; O=C(CCl)N1CC(O)C1]

Hydroxyazetidine hydrochloride (4.8 g, 43.8 mmol, 1 eq.) was added to a suspension of potassium carbonate (13.3 g, 96 mmol, 2.2 eq.) in water (32 mL) to form a reaction mixture. The reaction mixture is stirred at room temperature until complete dissolution, and then diluted with 35 mL of DCM and cooled to 0° C. prior to the dropwise introduction of chloroacetyl chloride (4.2 mL, 1.2 eq.) over 30 minutes. After 2 hours of stirring at room temperature, the reaction mixture is filtered, the organic layer is separated and saved, and the aqueous phase is extracted with a mixture of EtOAc and nBuOH (1:1) (6×16 mL), and the organic layer is obtained. The two organic layers were combined. The combined organic layers were dried over MgSO₄, filtered and concentrated in vacuo. The residue was suspended in acetone (48 mL) and stirred vigorously for 20 minutes and then was filtered. The filtrate was concentrated in vacuo to afford Compound 7, 2-chloro-1-(3-hydroxyazetidin-1-yl)ethan-1-one (4.2 g, 64%).

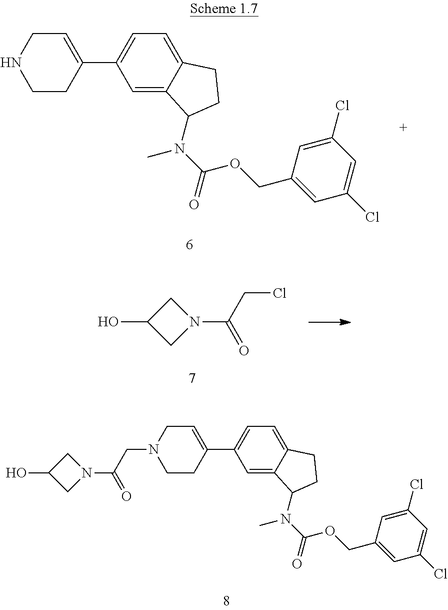 [SMILES: CN(C(=O)OCc1cc(Cl)cc(Cl)c1)C1CCc2ccc(C3=CCNCC3)cc21 ; O=C(CCl)N1CC(O)C1 ; CN(C(=O)OCc1cc(Cl)cc(Cl)c1)C1CCc2ccc(C3=CCN(CC(=O)N4CC(O)C4)CC3)cc21]

K₂CO₃ (106 mg, 2.0 eq.) and Compound 7 (2-chloro-1-(3-hydroxyazetidin-1-yl)ethan-1-one) (74 mg, 1.3 eq.) were added to a mixture of Compound 6 (3,5-dichlorobenzyl methyl(6-(1,2,3,6-tetrahydropyridin-4-yl)-2,3-dihydro-1H-inden-1-yl)carbamate) (165 mg, 0.3825 mmol) and dry MeCN (2.5 mL) at room temperature to form a reaction mixture. The reaction mixture was stirred at 80° C. for 5 hours and allowed to cool to room temperature. After that, the reaction mixture was stirred at room temperature overnight. TLC was used to confirm the completion of reaction. The solvent in the reaction mixture was removed, and then water was added to the reaction mixture. After that, the reaction mixture was extracted with EtOAc. The resulting organic phase was dried with Na₂SO₄ and concentrated under reduced pressure to give a crude mixture. The crude mixture was purified by column chromatography (MeOH/EtOAc=1/15 to MeOH/EtOAc=1/10) to give a white product. The white product was Compound 8, 3,5-dichlorobenzyl(6-(1-(2-(3-hydroxyazetidin-1-yl)-2-oxoethyl)-1,2,3,6-tetrahydropyridin-4-yl)-2,3-dihydro-1H-inden-1-yl)(methyl)carbamate (49 mg, 23.5%).

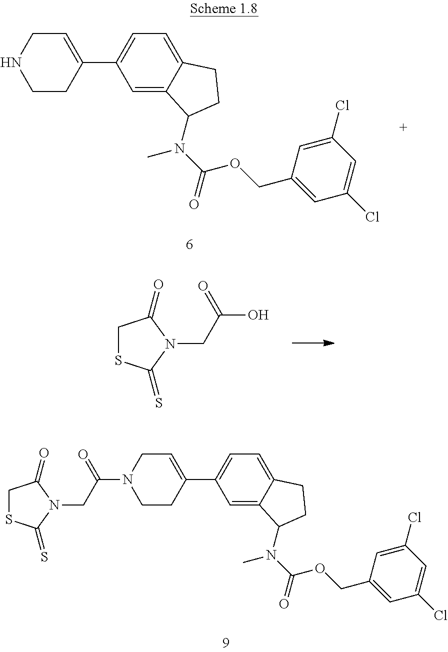 [SMILES: CN(C(=O)OCc1cc(Cl)cc(Cl)c1)C1CCc2ccc(C3=CCNCC3)cc21 ; O=C(O)CN1C(=O)CSC1=S ; CN(C(=O)OCc1cc(Cl)cc(Cl)c1)C1CCc2ccc(C3=CCN(C(=O)CN4C(=O)CSC4=S)CC3)cc21]

HOBt (35.5 mg, 0.5 eq.), EDC (133 mg, 1.5 eq.), NMM (0.1 mL, 2.0 eq.), and 4-oxo-2-thioxo-3-thiazolidinylacetic acid (133 mg, 1.5 eq.) were added to a mixture of Compound 6 (3,5-dichlorobenzyl methyl(6-(1,2,3,6-tetrahydropyridin-4-yl)-2,3-dihydro-1H-inden-1-yl)carbamate) (200 mg, 0.4637 mmol) and dry CH₂Cl₂ (6.0 mL). The reaction mixture was stirred at room temperature overnight. TLC was used to confirm the completion of reaction. After that, water was added to the mixture and the mixture was extracted with CH₂Cl₂. The resulting organic phase was dried with Na₂SO₄ and concentrated under reduced pressure to give a crude mixture. The crude mixture was purified by column chromatography (EtOAc/Hexanes=1/1 to EtOAc/Hexanes=2/1) to give an orange product. The orange product was Compound 9, 3,5-dichlorobenzyl methyl(6-(1-(2-(4-oxo-2-thioxothiazolidin-3-yl)acetyl)-1,2,3,6-tetrahydropyridin-4-yl)-2,3-dihydro-1H-inden-1-yl)carbamate (190 mg, 68%).

Spectral Data of Compound 8 and 9

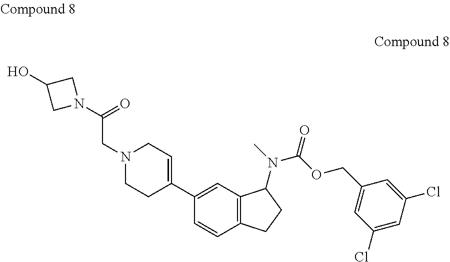 [SMILES: CN(C(=O)OCc1cc(Cl)cc(Cl)c1)C1CCc2ccc(C3=CCN(CC(=O)N4CC(O)C4)CC3)cc21]

Compound 8, ¹H-NMR (400 MHz, CDCl₃): 7.32-7.24 (m, 4H), 7.19 (d, 1H), 7.13 (d, 1H), 6.02-5.90 (m, 1H), 5.88-5.73 (m, 1H), 5.21-5.10 (m, 3H), 4.68 (br, 1H) 4.52-4.48 (m, 1H), 4.33-4.29 (m, 1H), 4.13 (dd, 1H), 3.91 (dd, 1H), 3.22 (s, 2H), 3.18 (s, 2H), 3.00-2.95 (m, 1H), 2.89-2.83 (m, 1H), 2.81-2.74 (m, 2H), 2.76 (d, 3H), 2.42 (s, 2H), 2.22 (s, 1H), 2.05-1.96 (m, 1H). ESI-MS m/z calcd for C₂₈H₃₁Cl₂N₃O₄ 543.17, found 544.3 [M+H]⁺.

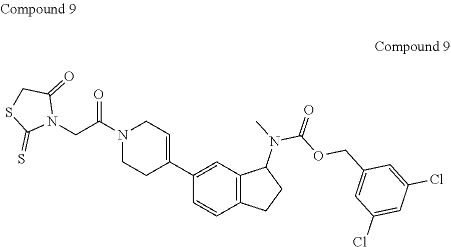 [SMILES: CN(C(=O)OCc1cc(Cl)cc(Cl)c1)C1CCc2ccc(C3=CCN(C(=O)CN4C(=O)CSC4=S)CC3)cc21]

Compound 9, ¹H-NMR (400 MHz, CDCl₃): 7.36-7.18 (m, 5H), 7.13-7.05 (m, 1H), 6.05-5.95 (m, 1H), 5.93-5.70 (m, 1H), 5.22-5.08 (m, 2H), 4.87 (d, 2H), 4.22 (s, 2H), 4.15-4.09 (m, 2H), 3.85-3.77 (m, 1H), 3.75-3.70 (m, 1H), 3.05-2.95 (m, 1H), 2.93-2.82 (m, 1H), 2.68 (d, 3H), 2.60-2.35 (m, 2H), 2.05-1.90 (m, 2H). ESI-MS m/z calcd for C₂₈H₂₇Cl₂N₃O₄S₂ 603.08, found 604.2 [M+H]⁺.

### Example 2. Compounds 10-30

Compounds 10-17 and 19-21 were produced by using the same methods of Schemes 1.1 to 1.7. Compounds 18, 22, 24-30 were produced by using the same methods of Schemes 1.1 to 1.6 and Scheme 1.8. Compound 23 was produced by using the same methods for Compound 10 by replacing chloroacetyl chloride with oxalyl chloride.

Spectral Data of Compounds 10-30

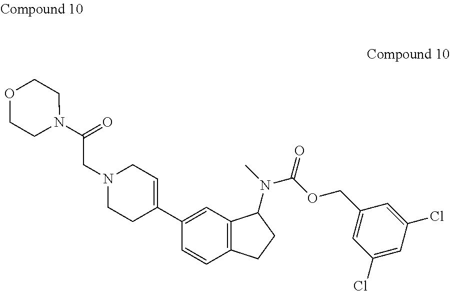 [SMILES: CN(C(=O)OCc1cc(Cl)cc(Cl)c1)C1CCc2ccc(C3=CCN(CC(=O)N4CCOCC4)CC3)cc21]

Compound 10, ¹H-NMR (400 MHz, CDCl₃): 7.32-7.24 (m, 4H), 7.19 (d, 1H), 7.13 (d, 1H), 6.02-5.90 (m, 1H), 5.89-5.71 (m, 1H), 5.21-5.10 (m, 2H), 3.83-3.55 (m, 8H), 3.32 (m, 2H), 3.23-3.19 (m, 2H), 3.02-2.95 (m, 1H), 2.90-2.83 (m, 1H), 2.77 (t, 1H), 2.71 (m, 1H), 2.70-2.62 (m, 3H), 2.57-2.48 (m, 2H), 2.53-2.41 (m, 1H), 2.01-1.93 (m, 1H). ESI-MS m/z calcd for C₂₉H₃₃Cl₂N₃O₄ 557.18, found 558.3 [M+H]⁺.

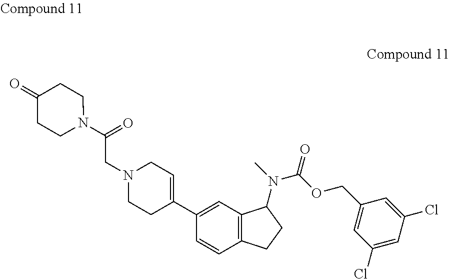 [SMILES: CN(C(=O)OCc1cc(Cl)cc(Cl)c1)C1CCc2ccc(C3=CCN(CC(=O)N4CCC(=O)CC4)CC3)cc21]

Compound 11, ¹H-NMR (400 MHz, CDCl₃): 7.31-7.26 (m, 4H), 7.19 (d, 1H), 7.13 (d, 1H), 6.02-5.90 (m, 1H), 5.85-5.70 (m, 1H), 5.21-5.09 (m, 2H), 3.91-3.87 (m, 4H), 3.49 (s, 2H), 3.31-3.23 (m, 2H), 3.06-2.92 (m, 1H), 2.89-2.83 (m, 1H), 2.79 (t, 2H), 2.70-2.69 (m, 4H), 2.58-2.44 (m, 5H), 2.43-2.38 (m, 1H), 2.04-1.89 (m, 1H). ESI-MS m/z calcd for C₃₀H₃₃Cl₂N₃O₄ 569.18, found 570.3 [M+H]⁺.

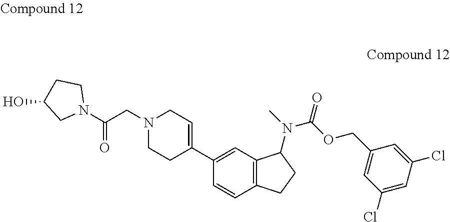 [SMILES: CN(C(=O)OCc1cc(Cl)cc(Cl)c1)C1CCc2ccc(C3=CCN(CC(=O)N4CC[C@@H](O)C4)CC3)cc21]

Compound 12, ¹H-NMR (300 MHz, CDCl₃): 7.32-7.09 (m, 6H), 6.05-5.98 (m, 1H), 5.95-5.70 (m, 1H), 5.15 (m, 2H), 4.58-4.49 (m, 1H), 3.72-3.52 (m, 5H), 3.35-3.27 (m, 3H), 3.06-2.92 (m, 1H), 2.90-2.78 (m, 2H), 2.70-2.62 (m, 4H), 2.61-2.51 (m, 2H), 2.48-2.32 (m, 1H), 2.19-1.89 (m, 4H). ESI-MS m/z calcd for C₂₉H₃₃Cl₂N₃O₄ 557.18, found 558.3 [M+H]⁺.

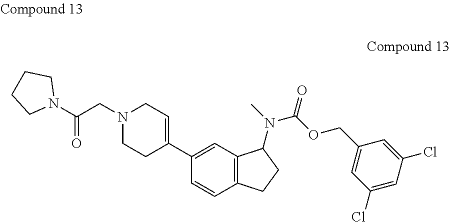 [SMILES: CN(C(=O)OCc1cc(Cl)cc(Cl)c1)C1CCc2ccc(C3=CCN(CC(=O)N4CCCC4)CC3)cc21]

Compound 13, ¹H-NMR (400 MHz, CDCl₃): 7.38-7.20 (m, 4H), 7.16 (d, 1H), 7.10 (d, 1H), 6.05-5.98 (m, 1H), 5.89-5.71 (m, 1H), 5.20-5.10 (m, 2H), 3.52-3.40 (m, 4H), 3.38 (s, 3H), 3.01-2.94 (m, 1H), 2.92-2.81 (m, 3H), 2.71-2.62 (m, 3H), 2.61-2.52 (m, 2H), 2.48-2.35 (m, 1H), 2.05-1.91 (m, 2H), 1.89-1.78 (m, 4H). ESI-MS m/z calcd for C₂₉H₃₃Cl₂N₃O₃ 541.19, found 542.3 [M+H]⁺.

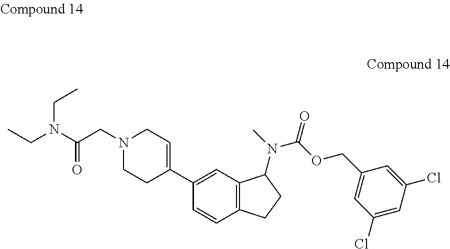 [SMILES: CCN(CC)C(=O)CN1CC=C(c2ccc3c(c2)C(N(C)C(=O)OCc2cc(Cl)cc(Cl)c2)CC3)CC1]

Compound 14, ¹H-NMR (300 MHz, CDCl₃): 7.32-7.22 (m, 4H), 7.18 (d, 1H), 7.14 (d, 1H), 6.02-5.97 (m, 1H), 5.90-5.84 (m, 1H), 5.21-5.09 (m, 2H), 3.45-3.34 (m, 4H), 3.29 (m, 2H), 3.25-3.22 (m, 2H), 3.02-2.91 (m, 1H), 2.90-2.84 (m, 1H), 2.80-2.77 (m, 2H), 2.70-2.61 (m, 3H), 2.59-2.50 (m, 2H), 2.48-2.28 (m, 1H), 2.09-1.88 (m, 1H), 1.27-1.16 (m, 6H). ESI-MS m/z calcd for C₂₉H₃₅Cl₂N₃O₃ 543.21, found 544.3 [M+H]⁺.

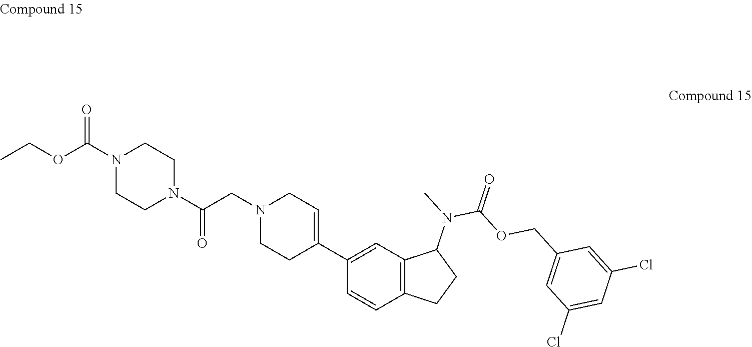 [SMILES: CCOC(=O)N1CCN(C(=O)CN2CC=C(c3ccc4c(c3)C(N(C)C(=O)OCc3cc(Cl)cc(Cl)c3)CC4)CC2)CC1]

Compound 15, ¹H-NMR (300 MHz, CDCl₃): 7.32-7.22 (m, 4H), 7.19 (d, 1H), 7.13 (d, 1H), 6.04-5.98 (m, 1H), 5.91-5.68 (m, 1H), 5.21-5.09 (m, 2H), 4.12 (q, 2H), 3.63-3.60 (m, 4H), 3.54-3.47 (m, 4H), 3.32 (s, 2H), 3.22-3.19 (m, 2H), 3.03-2.94 (m, 1H), 2.93-2.82 (m, 1H), 2.78 (t, 2H), 2.69-2.62 (m, 3H), 2.58-2.49 (m, 2H), 2.47-2.33 (m, 1H), 2.07-1.88 (m, 1H), 1.26 (t, 3H). ESI-MS m/z calcd for C₃₂H₃₈Cl₂N₄O₅ 628.22, found 629.3 [M+H]⁺.

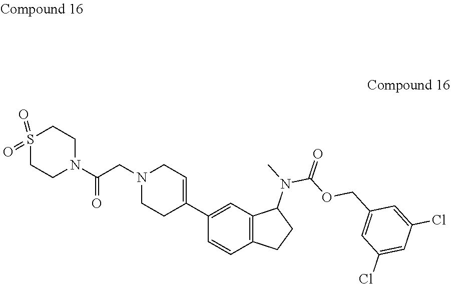 [SMILES: CN(C(=O)OCc1cc(Cl)cc(Cl)c1)C1CCc2ccc(C3=CCN(CC(=O)N4CCS(=O)(=O)CC4)CC3)cc21]

Compound 16, ¹H-NMR (300 MHz, CDCl₃): 7.33-7.22 (m, 4H), 7.20 (d, 1H), 7.13 (d, 1H), 6.04-5.98 (m, 1H), 5.92-5.69 (m, 1H), 5.22-5.09 (m, 2H), 4.16-4.05 (m, 4H), 3.37 (s, 2H), 3.21-3.16 (m, 2H), 3.12-3.01 (m, 4H), 3.00-2.80 (m, 3H), 2.76 (t, 1H), 2.74-2.66 (m, 3H), 2.60-2.49 (m, 2H), 2.47-2.32 (m, 1H), 2.08-1.92 (m, 1H). ESI-MS m/z calcd for C₂₉H₃₃Cl₂N₃O₅S 605.15, found 606.3 [M+H]⁺.

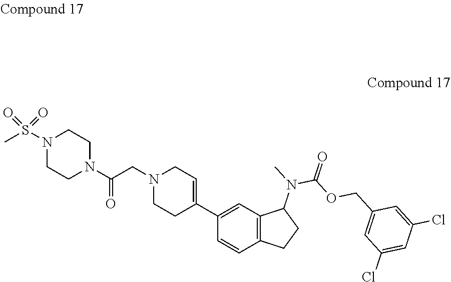 [SMILES: CN(C(=O)OCc1cc(Cl)cc(Cl)c1)C1CCc2ccc(C3=CCN(CC(=O)N4CCN(S(C)(=O)=O)CC4)CC3)cc21]

Compound 17, ¹H-NMR (400 MHz, CDCl₃): 7.33-7.21 (m, 4H), 7.19 (d, 1H), 7.13 (d, 1H), 6.04-5.98 (m, 1H), 5.90-5.70 (m, 1H), 5.22-5.08 (m, 2H), 3.82-3.68 (m, 4H), 3.33 (s, 2H), 3.27-3.14 (m, 6H), 3.03-2.93 (m, 1H), 2.91-2.82 (m, 1H), 2.78 (s, 3H), 2.76 (t, 2H), 2.70-2.65 (m, 3H), 2.57-2.48 (m, 2H), 2.46-2.35 (m, 1H), 2.05-1.90 (m, 1H). ESI-MS m/z calcd for C₃₀H₃₆Cl₂N₄O₅S 634.18, found 635.3 [M+H]⁺.

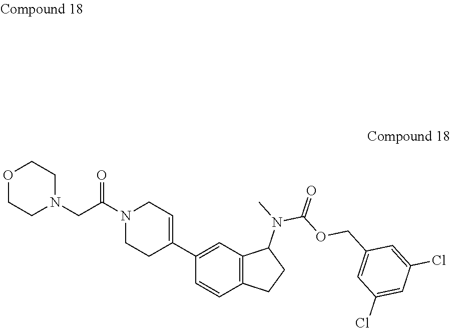 [SMILES: CN(C(=O)OCc1cc(Cl)cc(Cl)c1)C1CCc2ccc(C3=CCN(C(=O)CN4CCOCC4)CC3)cc21]

Compound 18, ¹H-NMR (400 MHz, CDCl₃): 7.34-7.19 (m, 5H), 7.16-7.08 (m, 1H), 6.09-5.94 (m, 1H), 5.92-5.70 (m, 1H), 5.22-5.09 (m, 2H), 4.21 (dd, 2H), 3.87-3.68 (m, 6H), 3.24 (d, 2H), 3.03-2.95 (m, 1H), 2.93-2.80 (m, 1H), 2.76 (d, 3H), 2.63-2.49 (m, 6H), 2.47-2.33 (m, 1H), 2.08-1.90 (m, 1H). ESI-MS m/z calcd for C₂₉H₃₃Cl₂N₃O₄ 557.18, found 558.3 [M+H]⁺.

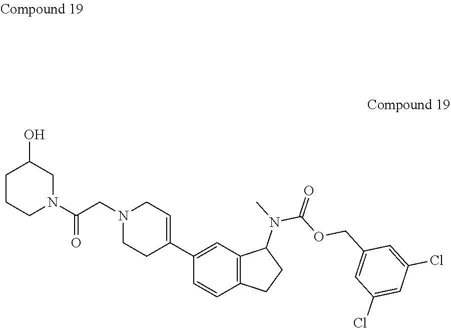 [SMILES: CN(C(=O)OCc1cc(Cl)cc(Cl)c1)C1CCc2ccc(C3=CCN(CC(=O)N4CCCC(O)C4)CC3)cc21]

Compound 19, ¹H-NMR (300 MHz, CDCl₃): 7.34-7.22 (m, 4H), 7.20-7.11 (m, 2H), 6.06-5.99 (m, 1H), 5.90-5.66 (m, 1H), 5.22-5.08 (m, 2H), 4.52 (d, 1H), 3.95 (s, 1H), 3.83 (d, 1H), 3.54 (d, 1H), 3.35-3.23 (m, 3H), 3.04-2.92 (m, 2H), 2.83 (t, 1H), 2.82-2.72 (m, 1H), 2.71-2.54 (m, 5H), 2.50-2.30 (m, 1H), 2.01-1.82 (m, 3H), 1.67-1.40 (m, 5H). ESI-MS m/z calcd for C₃₀H₃₅Cl₂N₃O₄ 571.20, found 572.3 [M+H]⁺.

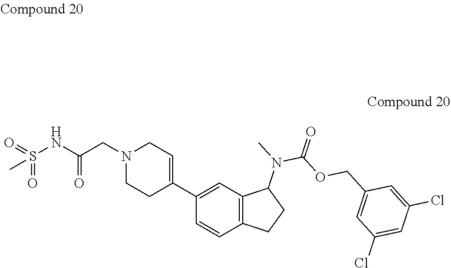 [SMILES: CN(C(=O)OCc1cc(Cl)cc(Cl)c1)C1CCc2ccc(C3=CCN(CC(=O)NS(C)(=O)=O)CC3)cc21]

Compound 20, ¹H-NMR (300 MHz, CDCl₃): 7.31-7.20 (m, 5H), 7.14 (s, 1H), 5.98 (s, 1H), 5.88 (t, 1H), 5.73 (s, 1H), 5.20-5.11 (m, 2H), 3.57-3.45 (m, 4H), 3.17 (m, 4H), 3.06-2.80 (m, 2H), 2.67 (m, 6H), 2.48-2.38 (m, 1H), 2.05-2.01 (m, 1H). ESI-MS m/z calcd for C₂₆H₂₉Cl₂N₃O₅S 565.12, found 566.2 [M+H]⁺.

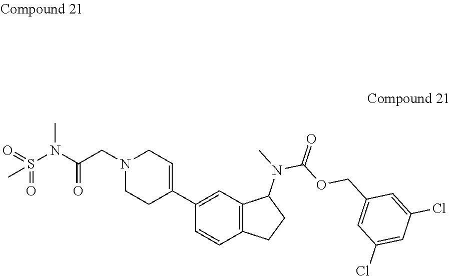 [SMILES: CN(C(=O)OCc1cc(Cl)cc(Cl)c1)C1CCc2ccc(C3=CCN(CC(=O)N(C)S(C)(=O)=O)CC3)cc21]

Compound 21, ¹H-NMR (300 MHz, CDCl₃): 7.29-7.23 (m, 5H), 7.14 (s, 1H), 6.00 (s, 1H), 5.87 (t, 1H), 5.22-5.08 (m, 2H), 4.70-4.65 (m, 1H), 4.43-4.32 (m, 2H), 4.13-3.96 (m, 1H), 3.66-3.49 (m, 4H), 3.13 (s, 3H), 3.03-2.84 (m, 2H), 2.67 (m, 6H), 2.42-2.27 (m, 1H), 2.08-2.04 (m, 1H). ESI-MS m/z calcd for C₂₇H₃₁Cl₂N₃O₅S 579.14, found 580.2 [M+H]⁺.

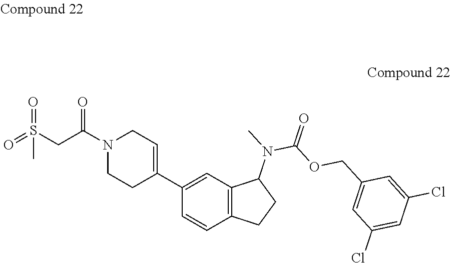 [SMILES: CN(C(=O)OCc1cc(Cl)cc(Cl)c1)C1CCc2ccc(C3=CCN(C(=O)CS(C)(=O)=O)CC3)cc21]

Compound 22, ¹H-NMR (400 MHz, CDCl₃): 7.34-7.19 (m, 5H), 7.11 (d, 1H), 6.05-5.93 (m, 1H), 5.92-5.68 (m, 1H), 5.21-5.10 (m, 2H), 4.30-4.26 (m, 2H), 4.12 (d, 2H), 3.87-3.78 (m, 2H), 3.15 (d, 3H), 3.08-2.95 (m, 1H), 2.93-2.78 (m, 1H), 2.67 (d, 4H), 2.58-2.35 (m, 2H), 2.04-1.90 (m, 1H). ESI-MS m/z calcd for C₂₆H₂₈Cl₂N₂O₅S 550.11, found 573.2 [M+Na]⁺.

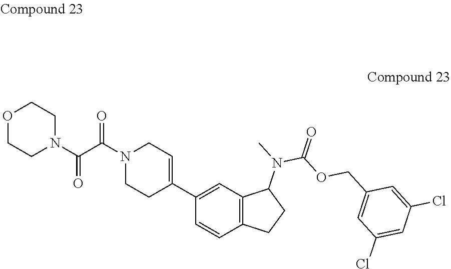 [SMILES: CN(C(=O)OCc1cc(Cl)cc(Cl)c1)C1CCc2ccc(C3=CCN(C(=O)C(=O)N4CCOCC4)CC3)cc21]

Compound 23, ¹H-NMR (300 MHz, CDCl₃): 7.32-7.09 (m, 6H), 6.04-5.98 (m, 1H), 5.90-5.72 (m, 1H), 5.22-5.10 (m, 2H), 4.27-4.10 (m, 2H), 3.87-3.85 (m, 1H), 3.76-3.64 (m, 7H), 3.49-3.43 (m, 2H), 3.03-2.83 (m, 2H), 2.69-2.60 (m, 5H), 2.45-2.17 (m, 2H). ESI-MS m/z calcd for C₂₉H₃₁Cl₂N₃O₅ 571.16, found 594.3[M+Na]⁺.

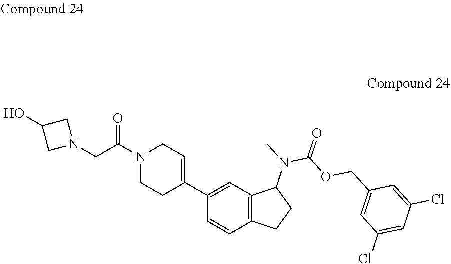 [SMILES: CN(C(=O)OCc1cc(Cl)cc(Cl)c1)C1CCc2ccc(C3=CCN(C(=O)CN4CC(O)C4)CC3)cc21]

Compound 24, ¹H-NMR (300 MHz, CDCl₃): 7.32-7.08 (m, 6H), 6.03-5.95 (m, 1H), 5.91-5.70 (m, 1H), 5.22-5.09 (m, 3H), 4.53-4.47 (m, 1H), 4.19-4.09 (m, 2H), 3.88-3.79 (m, 3H), 3.67-3.61 (m, 1H), 3.52-3.47 (m, 2H), 3.27-3.22 (m, 2H), 3.04-2.80 (m, 2H), 2.69-2.66 (m, 3H), 2.55-2.36 (m, 3H), 2.01-1.89 (m, 1H). ESI-MS m/z calcd for C₂₈H₃₁Cl₂N₃O₄ 543.17, found 544.3 [M+H]⁺.

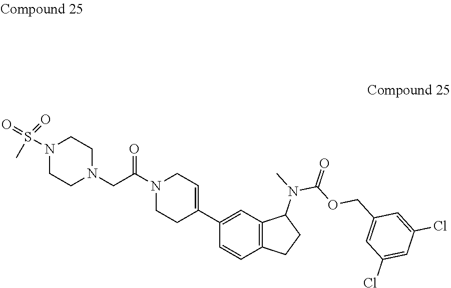 [SMILES: CN(C(=O)OCc1cc(Cl)cc(Cl)c1)C1CCc2ccc(C3=CCN(C(=O)CN4CCN(S(C)(=O)=O)CC4)CC3)cc21]

Compound 25, ¹H-NMR (400 MHz, CDCl₃): 7.32-7.09 (m, 6H), 6.05-5.98 (m, 1H), 5.90-5.72 (m, 1H), 5.23-5.09 (m, 2H), 4.22-4.18 (m, 2H), 3.83-3.70 (m, 2H), 3.29-3.24 (m, 6H), 3.04-2.85 (m, 3H), 2.81-2.79 (m, 3H), 2.69-2.67 (m, 6H), 2.64-2.52 (m, 3H), 2.01-1.90 (m, 1H). ESI-MS m/z calcd for C₃₀H₃₆Cl₂N₄O₅S 634.18, found 635.3 [M+H]⁺.

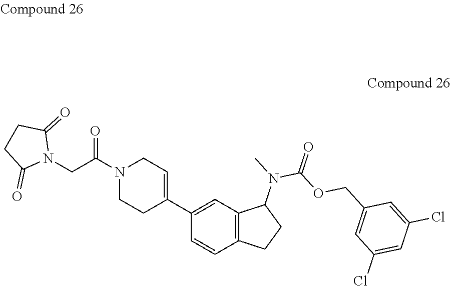 [SMILES: CN(C(=O)OCc1cc(Cl)cc(Cl)c1)C1CCc2ccc(C3=CCN(C(=O)CN4C(=O)CCC4=O)CC3)cc21]

Compound 26, ¹H-NMR (300 MHz, CDCl₃): 7.35-7.19 (m, 5H), 7.17-7.08 (m, 1H), 6.02-5.95 (m, 1H), 5.92-5.70 (m, 1H), 5.23-5.08 (m, 2H), 4.37 (d, 2H), 4.18 (d, 2H), 3.85-3.72 (m, 1H), 3.68 (t, 1H), 3.05-2.78 (m, 6H), 2.76-2.60 (m, 4H), 2.58-2.50 (m, 1H), 2.49-2.38 (m, 1H), 2.10-1.92 (m, 1H). ESI-MS m/z calcd for C₂₉H₂₉Cl₂N₃O₅ 569.15, found 592.2[M+Na]⁺.

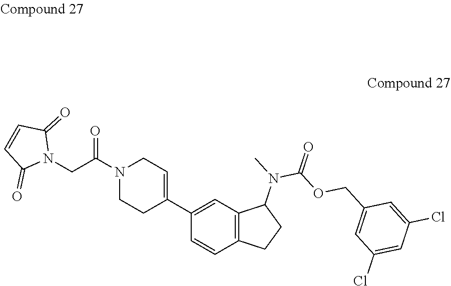 [SMILES: CN(C(=O)OCc1cc(Cl)cc(Cl)c1)C1CCc2ccc(C3=CCN(C(=O)CN4C(=O)C=CC4=O)CC3)cc21]

Compound 27, ¹H-NMR (400 MHz, CDCl₃): 7.34-7.20 (m, 5H), 7.16-7.08 (m, 1H), 6.80 (s, 2H), 6.03-5.96 (m, 1H), 5.91-5.72 (m, 1H), 5.22-5.10 (m, 2H), 4.42 (s, 1H), 4.37 (s, 1H), 4.14 (dd, 2H), 3.85-3.72 (m, 1H), 3.68 (t, 1H), 3.05-2.94 (m, 1H), 2.91-2.81 (m, 1H), 2.75-2.67 (m, 2H), 2.66-2.38 (m, 4H), 2.10-1.90 (m, 1H). ESI-MS m/z calcd for C₂₉H₂₇Cl₂N₃O₅ 567.13, found 590.3 [M+Na]⁺.

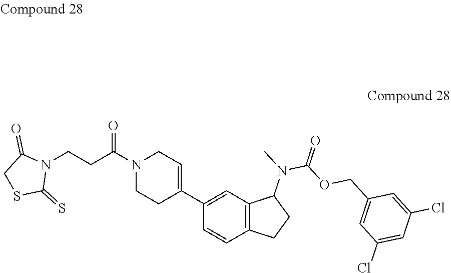 [SMILES: CN(C(=O)OCc1cc(Cl)cc(Cl)c1)C1CCc2ccc(C3=CCN(C(=O)CCN4C(=O)CSC4=S)CC3)cc21]

Compound 28, ¹H-NMR (400 MHz, CDCl₃): 7.36-7.20 (m, 5H), 7.13-7.08 (m, 1H), 6.04-5.95 (m, 1H), 5.90-5.71 (m, 1H), 5.21-5.09 (m, 2H), 4.40-4.33 (m, 2H), 4.21 (d, 1H), 4.10 (d, 1H), 4.07 (s, 1H), 3.96 (s, 1H), 3.88-3.72 (m, 1H), 3.63 (t, 1H), 3.02-2.92 (m, 1H), 2.90-2.81 (m, 1H), 2.78-2.71 (m, 2H), 2.70-2.63 (m, 3H), 2.61-2.48 (m, 2H), 2.47-2.32 (m, 1H), 2.08-1.90 (m, 1H). ESI-MS m/z calcd for C₂₉H₂₉Cl₂N₃O₄S₂ 617.10, found 618.2 [M+H]⁺.

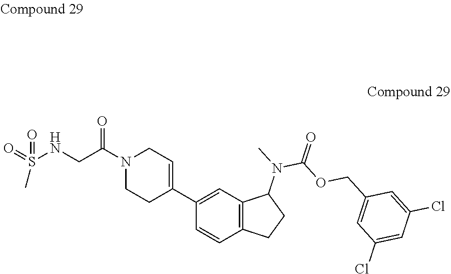 [SMILES: CN(C(=O)OCc1cc(Cl)cc(Cl)c1)C1CCc2ccc(C3=CCN(C(=O)CNS(C)(=O)=O)CC3)cc21]

Compound 29, ¹H-NMR (400 MHz, CDCl₃): 7.34-7.20 (m, 5H), 7.12-7.03 (m, 1H), 6.00-5.92 (m, 1H), 5.91-5.70 (m, 1H), 5.42-5.31 (m, 1H), 5.24-5.08 (m, 2H), 4.26 (s, 1H), 4.09-4.00 (m, 3H), 3.90-3.80 (m, 1H), 3.59 (t, 1H), 3.03-2.93 (m, 4H), 2.91-2.82 (m, 1H), 2.68 (d, 3H), 2.59-2.54 (m, 2H), 2.51-2.38 (m, 1H), 2.10-1.92 (m, 1H). ESI-MS m/z calcd for C₂₆H₂₉Cl₂N₃O₅S 565.12, found 588.2[M+Na]⁺.

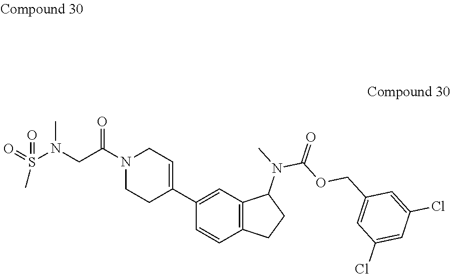 [SMILES: CN(C(=O)OCc1cc(Cl)cc(Cl)c1)C1CCc2ccc(C3=CCN(C(=O)CN(C)S(C)(=O)=O)CC3)cc21]

Compound 30, ¹H-NMR (400 MHz, CDCl₃): 7.37-7.21 (m, 5H), 7.13-7.10 (m, 1H), 6.05-5.95 (m, 1H), 5.91-5.72 (m, 1H), 5.22-5.09 (m, 2H), 4.21 (d, 2H), 4.20-4.19 (m, 1H), 4.07-4.06 (m, 1H), 3.85-3.75 (m, 1H), 3.62 (t, 1H), 3.07-3.01 (m, 6H), 3.00-2.96 (m, 1H), 2.91-2.83 (m, 1H), 2.69-2.67 (m, 3H), 2.62-2.50 (m, 2H), 2.50-2.33 (m, 1H), 2.05-1.95 (m, 1H). ESI-MS m/z calcd for C₂₇H₃₁Cl₂N₃O₅S 579.14, found 580.2[M+H]⁺.

### Example 3. Compounds 33 and 34

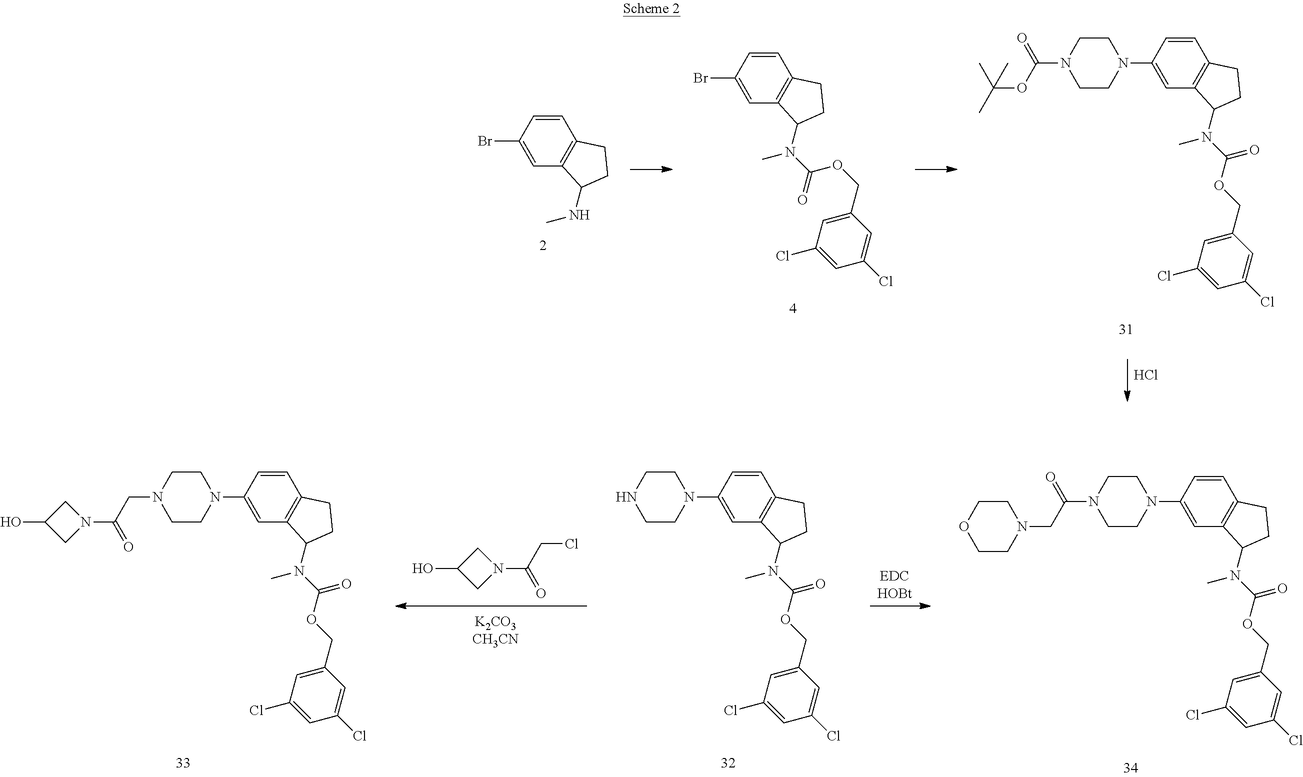

For the preparation of Compounds 33 and 34, please refer to Scheme 2 and the following details:

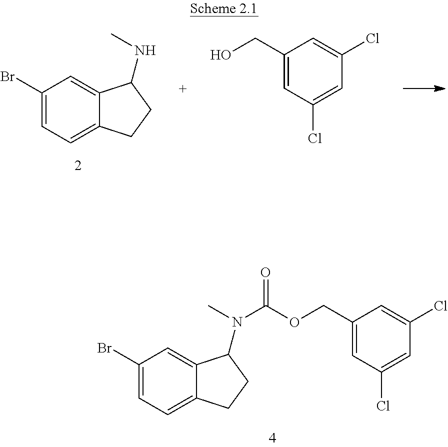 [SMILES: CNC1CCc2ccc(Br)cc21 ; OCc1cc(Cl)cc(Cl)c1 ; CN(C(=O)OCc1cc(Cl)cc(Cl)c1)C1CCc2ccc(Br)cc21]

DIPEA (2.06 g, 15.93 mmol) and triphosgene (1.89 g, 6.37 mmol) were added to a solution of 3,5-dichlorobenzyl alcohol (2.82 g, 15.93 mmol) in DCM (100 mL) at 0° C. The reaction mixture was stirred at the same temperature for 30 minutes. After the 3,5-dichlorobenzyl alcohol was consumed, a solution of Compound 2 (6-bromo-N-methyl-2,3-dihydro-1H-inden-1-amine) (3.00 g, 13.27 mmol) and DIPEA (2.06 g, 15.93 mmol) in DCM (30 mL) was added into the reaction mixture. Then the reaction mixture was slowly warmed to room temperature and stirred overnight. After the reaction was completed, the solvent in the reaction mixture was removed under reduced pressure. The residue was diluted with saturated NH₄Cl and extracted with EtOAc, and then resulting organic layers were combined. The combined organic layers were dried over MgSO₄ and concentrated in vacuo to obtain a crude product. The crude product was used as Compound 4 in the next step without further purification, and the yield thereof was 5.06 g (11.79 mmol).

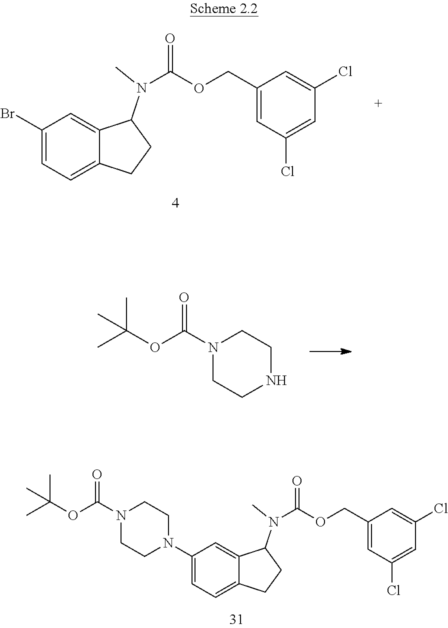 [SMILES: CN(C(=O)OCc1cc(Cl)cc(Cl)c1)C1CCc2ccc(Br)cc21 ; CC(C)(C)OC(=O)N1CCNCC1 ; CN(C(=O)OCc1cc(Cl)cc(Cl)c1)C1CCc2ccc(N3CCN(C(=O)OC(C)(C)C)CC3)cc21]

CsCO₃ (0.15 g, 0.45 mmol), Boc-piperazine (0.08 g, 0.45 mmol), 2-(di-t-butylphosphino)biphenyl (0.01 g, 0.03 mmol) and Pd(OAc)₂ (7.0 mg, 0.03 mmol) were added to a solution of Compound 4 (3,5-dichlorobenzyl (6-bromo-2,3-dihydro-1H-inden-1-yl)(methyl)carbamate) (0.13 g, 0.30 mmol) in toluene (5 mL). The mixture was degassed with argon for 15 minutes, and then heated to reflux overnight. After the reaction was completed, the solvent in the mixture was removed under reduced pressure, and then the residue was filtered through celite and washed with EtOAc. After concentration in vacuo, a resulting crude product was purified via flash column chromatography on a silica gel column using 4:1 hexane-EtOAc as the eluent to give Compound 31, tert-butyl 4-(3-((((3,5-dichlorobenzyl)oxy)carbonyl)(methyl)amino)-2,3-dihydro-1H-inden-5-yl)piperazine-1-carboxylate, and the yield thereof was 0.12 g (0.22 mmol).

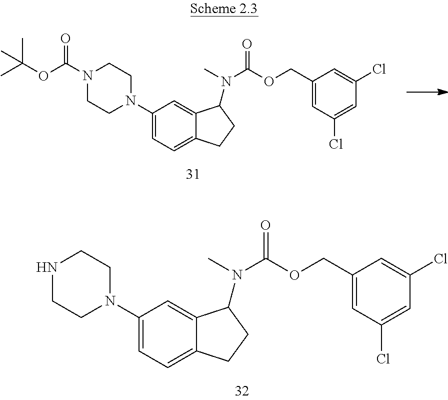 [SMILES: CN(C(=O)OCc1cc(Cl)cc(Cl)c1)C1CCc2ccc(N3CCN(C(=O)OC(C)(C)C)CC3)cc21 ; CN(C(=O)OCc1cc(Cl)cc(Cl)c1)C1CCc2ccc(N3CCNCC3)cc21]

The Compound 31 (tert-butyl 4-(3-((((3,5-dichlorobenzyl)oxy)carbonyl)(methyl)amino)-2,3-dihydro-1H-inden-5-yl)piperazine-1-carboxylate) (0.12 g, 0.22 mmol) was added to a solution of 4N HCl in dioxane (5 mL) to form a mixture, and then stirred for 3 hours. After the reaction was completed, the solvent in the mixture was removed under reduced pressure, and then a crude product was obtained. The crude product was used as Compound 32 (3,5-dichlorobenzyl methyl(6-(piperazin-1-yl)-2,3-dihydro-1H-inden-1-yl)carbamate) (yield, 0.10 g) in the next step without further purification.

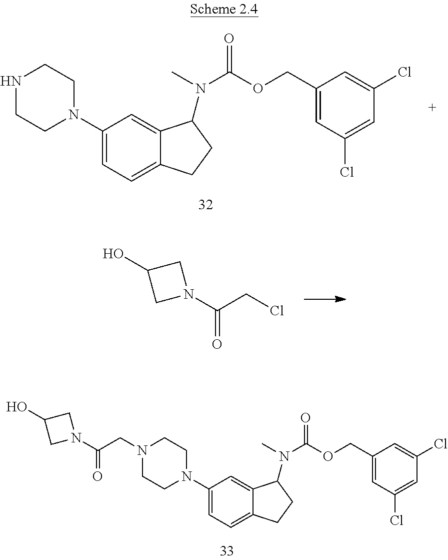 [SMILES: CN(C(=O)OCc1cc(Cl)cc(Cl)c1)C1CCc2ccc(N3CCNCC3)cc21 ; O=C(CCl)N1CC(O)C1 ; CN(C(=O)OCc1cc(Cl)cc(Cl)c1)C1CCc2ccc(N3CCN(CC(=O)N4CC(O)C4)CC3)cc21]

K₂CO₃ (0.18 g, 1.3 mmol), 2-chloro-1-(3-hydroxyazetidin-1-yl)ethan-1-one (0.04, 0.28 mmol) and a catalytic amount of KI were added to a solution of Compound 32 (3,5-dichlorobenzyl methyl(6-(piperazin-1-yl)-2,3-dihydro-1H-inden-1-yl)carbamate) (0.10 g) in CH₃CN (5 mL), and then the reaction mixture was heated to reflux overnight. After reaction was completed, the solvent in the reaction mixture was removed under reduced pressure. The residue was diluted with water and extracted with EtOAc, and the resulting organic layers were combined. The combined organic layers were dried over MgSO₄ and concentrated in vacuo to obtain a crude product. The crude product was purified via flash column chromatography on a silica gel column using 10:1 DCM-MeOH as the eluent to give Compound 33, 3,5-dichlorobenzyl (6-(4-(2-(3-hydroxyazetidin-1-yl)-2-oxoethyl)piperazin-1-yl)-2,3-dihydro-1H-inden-1-yl)(methyl)carbamate, and the yield thereof was 0.06 g (0.11 mmol).

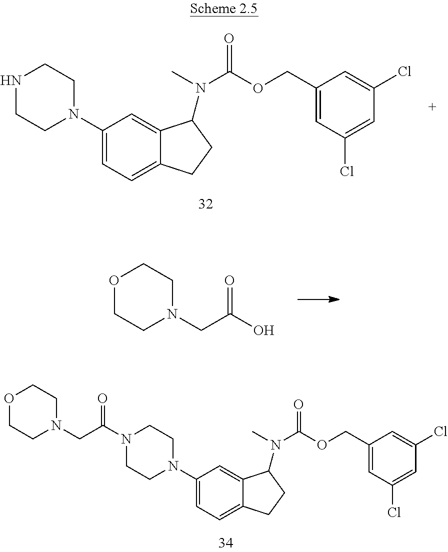 [SMILES: CN(C(=O)OCc1cc(Cl)cc(Cl)c1)C1CCc2ccc(N3CCNCC3)cc21 ; O=C(O)CN1CCOCC1 ; CN(C(=O)OCc1cc(Cl)cc(Cl)c1)C1CCc2ccc(N3CCN(C(=O)CN4CCOCC4)CC3)cc21]

NMM (0.159 g, 1.58 mmol) and EDCI (0.11 g, 0.59 mmol) were added to a mixture of Compound 32 (3,5-dichlorobenzyl methyl(6-(piperazin-1-yl)-2,3-dihydro-1H-inden-1-yl)carbamate) (0.2 g, 0.39 mmol), morpholin-4-ylacetic acid (0.09 g, 0.59 mmol), HOBt (0.01 g, 0.08 mmol) in DCM (20 mL) at 0° C. After addition, the reaction mixture was slowly warmed to room temperature and stirred overnight. After the reaction was completed, the solvent in the reaction mixture was removed under reduced pressure. The residue was diluted with saturated NH₄Cl and extracted with EtOAc, and the resulting organic layers were combined. The combined organic layers were dried over MgSO₄ and concentrated in vacuo. The crude product was purified via flash column chromatography on a silica gel column using 4:1 DCM-EtOAc as the eluent to give Compound 34, 3,5-dichlorobenzyl methyl(6-(4-(2-morpholinoacetyl)piperazin-1-yl)-2,3-dihydro-1H-inden-1-yl)carbamate, and the yield thereof was 0.19 g (0.34 mmol).

Spectral data of Compounds 33 and 34

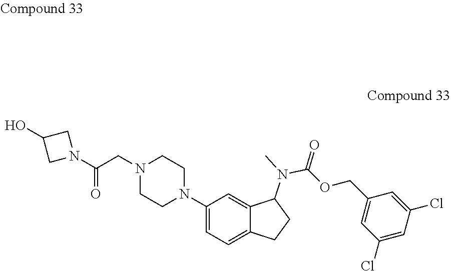 [SMILES: CN(C(=O)OCc1cc(Cl)cc(Cl)c1)C1CCc2ccc(N3CCN(CC(=O)N4CC(O)C4)CC3)cc21]

Compound 33, ¹H-NMR (400 MHz, CDCl₃): 7.31-7.24 (m, 3H), 7.14-7.12 (d, 1H), 6.85-6.83 (d, 1H), 6.70-6.66 (d, 1H), 5.85-5.68 (dt, 1H), 5.22-5.04 (m, 3H), 4.70-4.66 (m, 1H), 4.50-4.46 (m, 1H), 4.32-4.28 (m, 1H), 4.15-4.12 (m, 1H), 3.94-3.90 (m, 1H), 3.19-3.18 (m, 4H), 3.13-3.12 (d, 2H), 2.95-2.77 (m, 2H), 2.76-2.73 (m, 4H), 2.68-2.65 (d, 3H), 2.42-2.37 (m, 1H), 2.05-1.91 (m, 1H), ESI-MS m/z calcd for C₂₇H₃₂Cl₂N₄O₄ 546.18, found 547.3 [M+H]⁺.

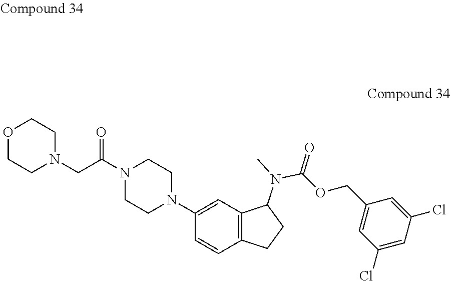 [SMILES: CN(C(=O)OCc1cc(Cl)cc(Cl)c1)C1CCc2ccc(N3CCN(C(=O)CN4CCOCC4)CC3)cc21]

Compound 34, ¹H-NMR (300 MHz, CDCl₃): 7.32-7.24 (m, 3H), 7.16-7.13 (m, 1H), 6.86-6.83 (m, 1H), 6.70-6.67 (m, 1H), 5.87-5.68 (m, 1H), 5.24-5.09 (m, 2H), 3.78-3.71 (m, 8H), 3.22 (s, 2H), 3.11-3.09 (m, 4H), 3.00-2.75 (m, 2H), 2.69-2.67 (m, 3H), 2.55-2.52 (m, 4H), 2.44-2.37 (m, 1H), 2.09-1.92 (m, 1H). ESI-MS m/z calcd for C₂₈H₃₄Cl₂N₄O₄ 560.20, found 561.3 [M+H]⁺.

### Example 4. Compounds 36-38

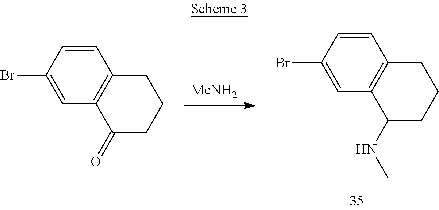

A solution of 7-bromo-1-tetralone (1.5 g, 6.66 mmol) in 30 mL of MeOH was treated with methyl amine (6.8 mL, 66.6 mmol). After 5 minutes, acetic acid (0.15 mL) was added followed by sodium cyanoborohydride (0.5 g, 8.0 mmol). The reaction mixture was stirred overnight at ambient temperature. MeOH and excess methyl amine in the reaction mixture were removed by vacuo. After that, water and saturated aqueous sodium carbonate solution were added to the reaction mixture followed by extraction with dichloromethane (×2). The organic layers were combined and dried over anhydrous sodium sulfate, filtered and evaporated in vacuo to obtain oil. The oil was purified via flash column chromatography over silica gel using 50% ethyl acetate in hexane as the eluent to afford Compound 35, 7-bromo-N-methyl-1,2,3,4-tetrahydronaphthalen-1-amine (1.0 g, 62%, 4.17 mmol).

Compounds 36-37 were produced by using the same methods for Compound 34. Compound 38 was produced by using the same methods for Compound 33 by replacing Compound 2 with Compound 35 in the corresponding schemes.

Spectral Data of Compounds 36-38

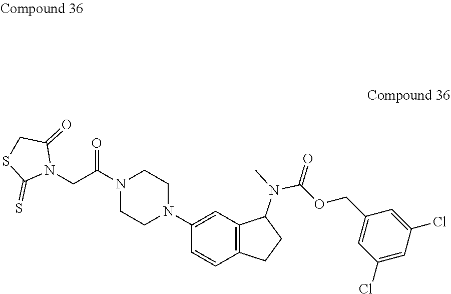 [SMILES: CN(C(=O)OCc1cc(Cl)cc(Cl)c1)C1CCc2ccc(N3CCN(C(=O)CN4C(=O)CSC4=S)CC3)cc21]

Compound 36, ¹H-NMR (300 MHz, CDCl₃): 7.32-7.25 (m, 3H), 7.17-7.14 (m, 1H), 6.87-6.83 (m, 1H), 6.71-6.67 (m, 1H), 5.87-5.68 (m, 1H), 5.24-5.08 (m, 2H), 4.86 (s, 2H), 4.10 (s, 2H), 3.76-3.67 (m, 4H), 3.21-3.12 (m, 4H), 2.99-2.75 (m, 2H), 2.69-2.67 (m, 3H), 2.44-2.37 (m, 1H), 2.04-1.92 (m, 1H). ESI-MS m/z calcd for C₂₇H₂₈Cl₂N₄O₄S₂ 606.09, found 607.2 [M+H]⁺

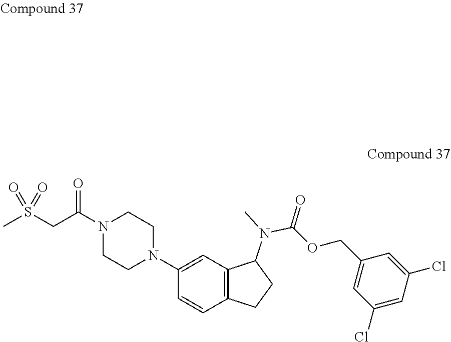 [SMILES: CN(C(=O)OCc1cc(Cl)cc(Cl)c1)C1CCc2ccc(N3CCN(C(=O)CS(C)(=O)=O)CC3)cc21]

Compound 37, ¹H-NMR (400 MHz, CDCl₃): 7.32-7.24 (m, 3H), 7.16-7.14 (m, 1H), 6.86-6.83 (m, 1H), 6.70-6.67 (m, 1H), 5.86-5.68 (m, 1H), 5.22-5.09 (m, 2H), 4.14-4.10 (m, 2H), 3.84-3.75 (m, 4H), 3.21-3.14 (m, 7H), 2.97-2.76 (m, 2H), 2.69-2.66 (m, 3H), 2.48-2.35 (m, 1H), 2.05-1.93 (m, 1H). ESI-MS m/z calcd for C₂₅H₂₉Cl₂N₃O₅S 553.12, found 554.2 [M+H]⁺.

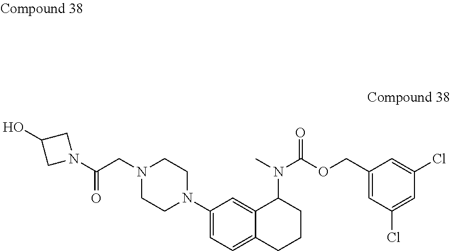 [SMILES: CN(C(=O)OCc1cc(Cl)cc(Cl)c1)C1CCCc2ccc(N3CCN(CC(=O)N4CC(O)C4)CC3)cc21]

Compound 38, ¹H-NMR (300 MHz, CDCl₃): 7.35-7.21 (m, 3H), 7.02-6.99 (d, 1H), 6.80-6.76 (m, 1H), 5.50-5.28 (m, 1H), 5.26-5.06 (m, 3H), 4.76-4.65 (m, 1H), 4.55-4.41 (m, 1H), 4.34-4.24 (m, 1H), 4.16-4.06 (m, 1H), 3.95-3.88 (m, 1H), 3.13-3.08 (m, 6H), 2.69-2.63 (m, 10H), 2.05-1.95 (m, 2H), 1.87-1.68 (m, 2H). ESI-MS m/z calcd for C₂₈H₃₄Cl₂N₄O₄ 560.20, found 561.3 [M+H]⁺.

### Example 5. Compounds 43 and 44

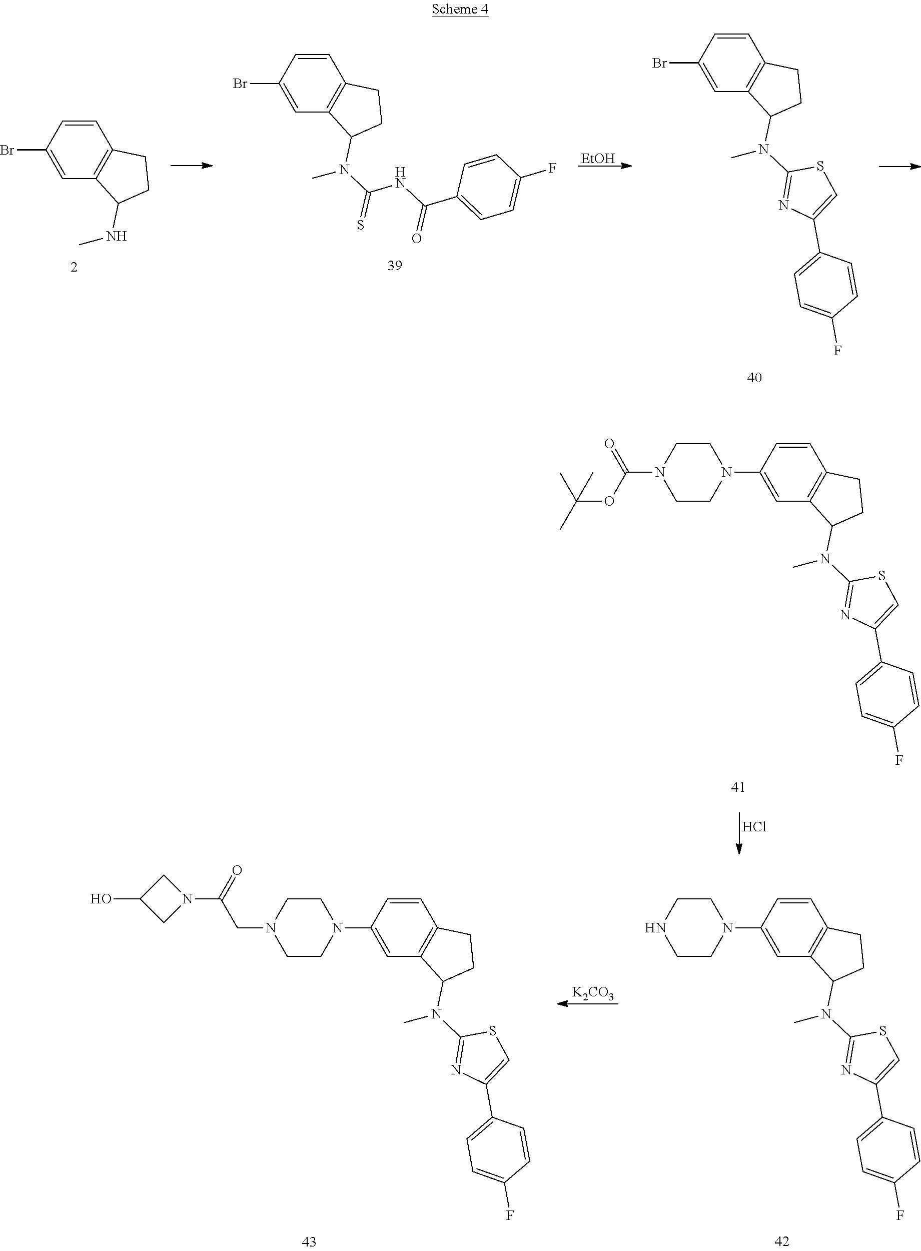

For the preparation of Compounds 43 and 44, please refer to Scheme 4 and the following details:

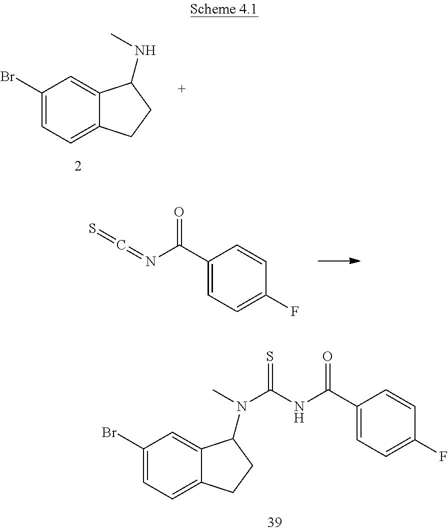 [SMILES: CNC1CCc2ccc(Br)cc21 ; O=C(N=C=S)c1ccc(F)cc1 ; CN(C(=S)NC(=O)c1ccc(F)cc1)C1CCc2ccc(Br)cc21]

4-fluorobenzoyl isothiocyanate (0.28 g, 1.54 mmol) was added to a solution of Compound 2 (6-bromo-N-methyl-2,3-dihydro-1H-inden-1-amine) (0.29 g, 1.28 mmol) in ACN (10 mL) and stirred for 3 hours at room temperature to form a mixture. After reaction was completed, the solvent in the mixture was removed under reduced pressure. The residual was diluted with cold water and sit to get a precipitated solid. The precipitated solid was collected by filtration and washed with ether to afford a crude product. The crude product was used as Compound 39, N-((6-bromo-2,3-dihydro-1H-inden-1-yl)(methyl)carbamothioyl)-4-fluorobenzamide (0.44 g, 1.07 mmol), in the next step without further purification.

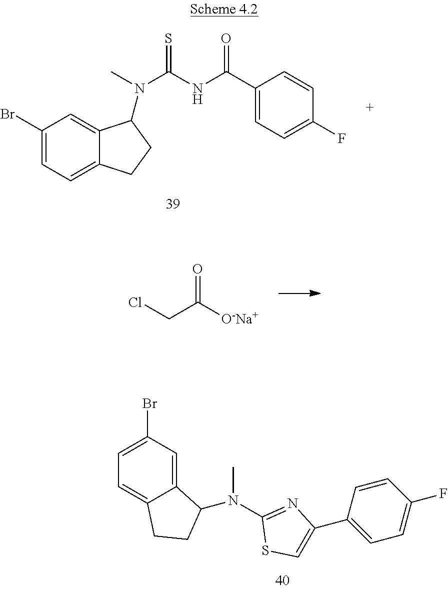

1N NaOH (3 mL) was added to a mixture of Compound 39 (N-((6-bromo-2,3-dihydro-1H-inden-1-yl)(methyl)carbamothioyl)-4-fluorobenzamide) (0.44 g, 1.07 mmol) and sodium chloroacetate (0.25 g, 2.14 mmol) in MeCN (10 mL) to form a reaction mixture, and then the reaction mixture was heated to reflux for 6 hours. After the reaction was completed, the solvent in the reaction mixture was removed under reduced pressure. The residue was diluted with saturated NaHCO₃ and extracted with EtOAc and the resulting organic layers were combined. The combined organic layers were dried over MgSO₄ and concentrated in vacuo to obtain a crude product. The crude product was purified via flash column chromatography on a silica gel column using 10:1 Hexane-EtOAc as the eluent to give Compound 40, N-(6-bromo-2,3-dihydro-1H-inden-1-yl)-4-(4-fluorophenyl)-N-methylthiazol-2-amine (0.29 g, 0.72 mmol).

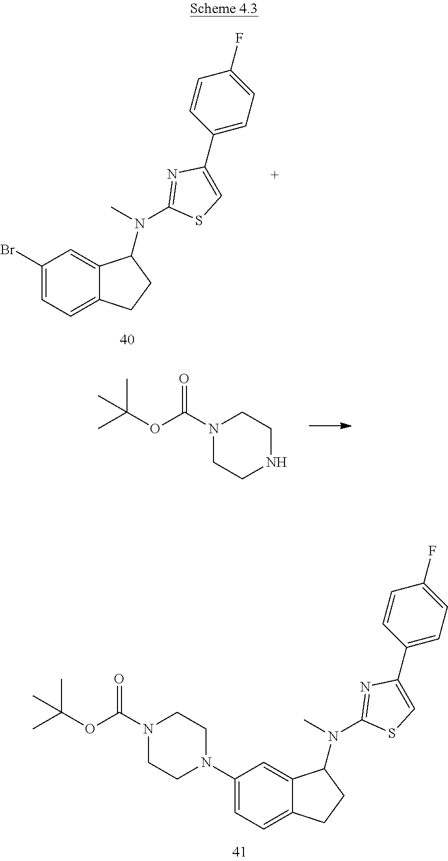 [SMILES: CN(c1nc(-c2ccc(F)cc2)cs1)C1CCc2ccc(Br)cc21 ; CC(C)(C)OC(=O)N1CCNCC1 ; CN(c1nc(-c2ccc(F)cc2)cs1)C1CCc2ccc(N3CCN(C(=O)OC(C)(C)C)CC3)cc21]

CsCO₃ (0.35 g, 1.08 mmol), Boc-piperazine (0.20 g, 1.08 mmol), 2-(di-t-butylphosphino)biphenyl (0.02 g, 0.07 mmol) and Pd(OAc)₂ (0.02 g, 0.07 mmol) were added to a solution of Compound 40 (N-(6-bromo-2,3-dihydro-1H-inden-1-yl)-4-(4-fluorophenyl)-N-methylthiazol-2-amine) (0.29 g, 0.72 mmol) in toluene (10 mL) to form a mixture. The mixture was degassed with argon for 15 minutes, and then heated to reflux overnight. After the reaction was completed, the solvent in the mixture was removed under reduced pressure, and then the residue was filtered through celite and washed with EtOAc to obtain a crude product. After concentration in vacuo, the crude product was purified via flash column chromatography on a silica gel column using 10:1 Hexane-EtOAc as the eluent to give Compound 41, tert-butyl 4-(3-((4-(4-fluorophenyl)thiazol-2-yl)(methyl)amino)-2,3-dihydro-1H-inden-5-yl)piperazine-1-carboxylate (0.30 g, 0.59 mmol).

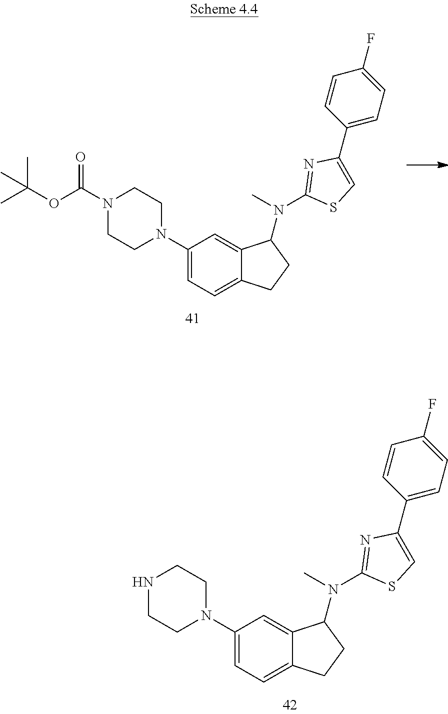 [SMILES: CN(c1nc(-c2ccc(F)cc2)cs1)C1CCc2ccc(N3CCN(C(=O)OC(C)(C)C)CC3)cc21 ; CN(c1nc(-c2ccc(F)cc2)cs1)C1CCc2ccc(N3CCNCC3)cc21]

Compound 41 (Tert-butyl 4-(3-((4-(4-fluorophenyl)thiazol-2-yl)(methyl)amino)-2,3-dihydro-1H-inden-5-yl)piperazine-1-carboxylate) (0.30 g, 0.59 mmol) was added to a solution of 4N HCl in dioxane (5 mL), and then stirred for 3 hours to form a mixture. After the reaction was completed, the solvent in the mixture was removed under reduced pressure to obtain a crude product. The crude product was used as Compound 42 in the next step without further purification.

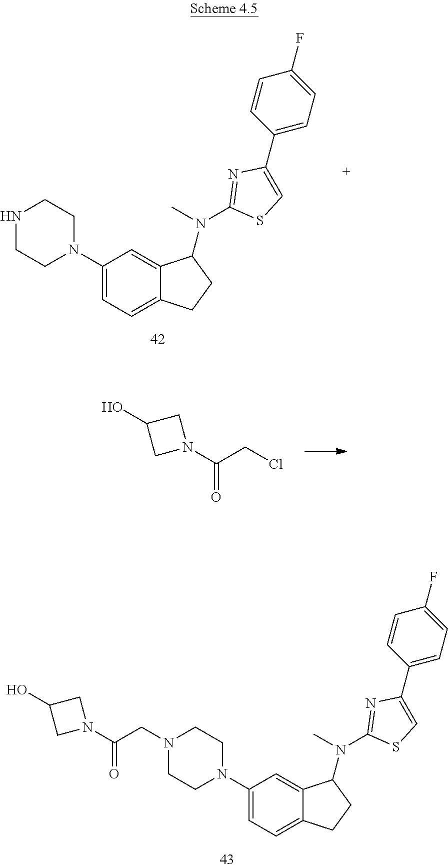 [SMILES: CN(c1nc(-c2ccc(F)cc2)cs1)C1CCc2ccc(N3CCNCC3)cc21 ; O=C(CCl)N1CC(O)C1 ; CN(c1nc(-c2ccc(F)cc2)cs1)C1CCc2ccc(N3CCN(CC(=O)N4CC(O)C4)CC3)cc21]

K₂CO₃ (0.49 g, 3.52 mmol), 2-chloro-1-(3-hydroxyazetidin-1-yl)ethan-1-one (0.09 g, 0.59 mmol) and a catalytic amount of KI were added to a solution of Compound 42 (4-(4-fluorophenyl)-N-methyl-N-(6-(piperazin-1-yl)-2,3-dihydro-1H-inden-1-yl)thiazol-2-amine) (0.5 mmol) in DMF (5 mL), and then the reaction mixture was heated to 80° C. overnight to obtain a mixture. After the reaction was completed, the solvent in the mixture was evaporated off by air-drying. The residue was diluted with water and extracted with EtOAc, and the resulting organic layers were combined. The combined organic layers were dried over MgSO₄ and concentrated in vacuo to obtain a crude product. The crude product was purified via flash column chromatography on a silica gel column using 10:1 DCM-MeOH as the eluent to give Compound 43, 2-(4-(3-((4-(4-fluorophenyl)thiazol-2-yl)(methyl)amino)-2,3-dihydro-1H-inden-5-yl)piperazin-1-yl)-1-(3-hydroxyazetidin-1-yl)ethan-1-one (0.23 g, 0.45 mmol).

Compound 44 was produced by using the same methods for Compound 43.

Spectral Data of Compounds 43 and 44

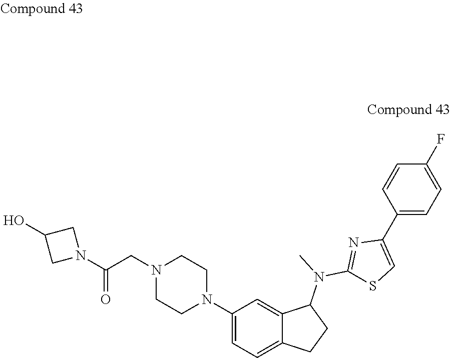 [SMILES: CN(c1nc(-c2ccc(F)cc2)cs1)C1CCc2ccc(N3CCN(CC(=O)N4CC(O)C4)CC3)cc21]

Compound 43, ¹H-NMR (400 MHz, CDCl₃): 7.86-7.82 (m, 2H), 7.17-7.15 (d, 1H), 7.08-7.03 (m, 2H), 6.88-6.85 (dd, 1H), 6.78 (d, 1H), 6.66 (s, 1H), 5.84-5.80 (t, 1H), 4.64-4.60 (m, 1H), 4.45-4.41 (m, 1H), 4.27-4.23 (m, 1H), 4.09-4.05 (m, 1H), 3.89-3.85 (m, 1H), 3.16-3.14 (m, 4H), 3.06 (s, 2H), 3.00-2.77 (m, 5H), 2.65-2.62 (m, 4H), 2.57-2.49 (m, 1H), 2.08-1.99 (m, 1H). 1.67 (br, 1H). ESI-MS m/z calcd for C₂₈H₃₂FN₅O₂S 521.23, found 522.3 [M+H]⁺.

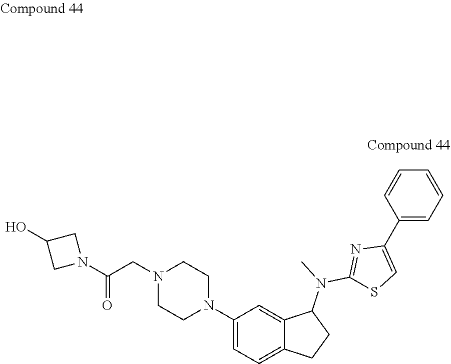 [SMILES: CN(c1nc(-c2ccccc2)cs1)C1CCc2ccc(N3CCN(CC(=O)N4CC(O)C4)CC3)cc21]

Compound 44, ¹H-NMR (300 MHz, CDCl₃): 7.88-7.86 (d, 2H), 7.39-7.36 (t, 2H), 7.29-7.27 (d, 2H), 7.17-7.15 (d, 1H), 6.87-6.85 (dd, 1H), 6.80 (s, 1H), 6.21 (s, 1H), 5.86-5.82 (t, 1H), 4.64-4.60 (m, 1H), 4.44-4.40 (m, 1H), 4.27-4.23 (m, 1H), 4.09-4.05 (m, 1H), 3.88-3.85 (m, 1H), 3.17-3.14 (m, 4H), 3.06 (s, 2H), 3.00-2.75 (m, 5H), 2.64-2.62 (m, 4H), 2.57-2.49 (m, 1H), 2.09-1.99 (m, 1H). ESI-MS m/z calcd for C₂₈H₃₃N₅O₂S 503.24, found 504.3 [M+H]⁺

### Example 6. Compounds 50-54

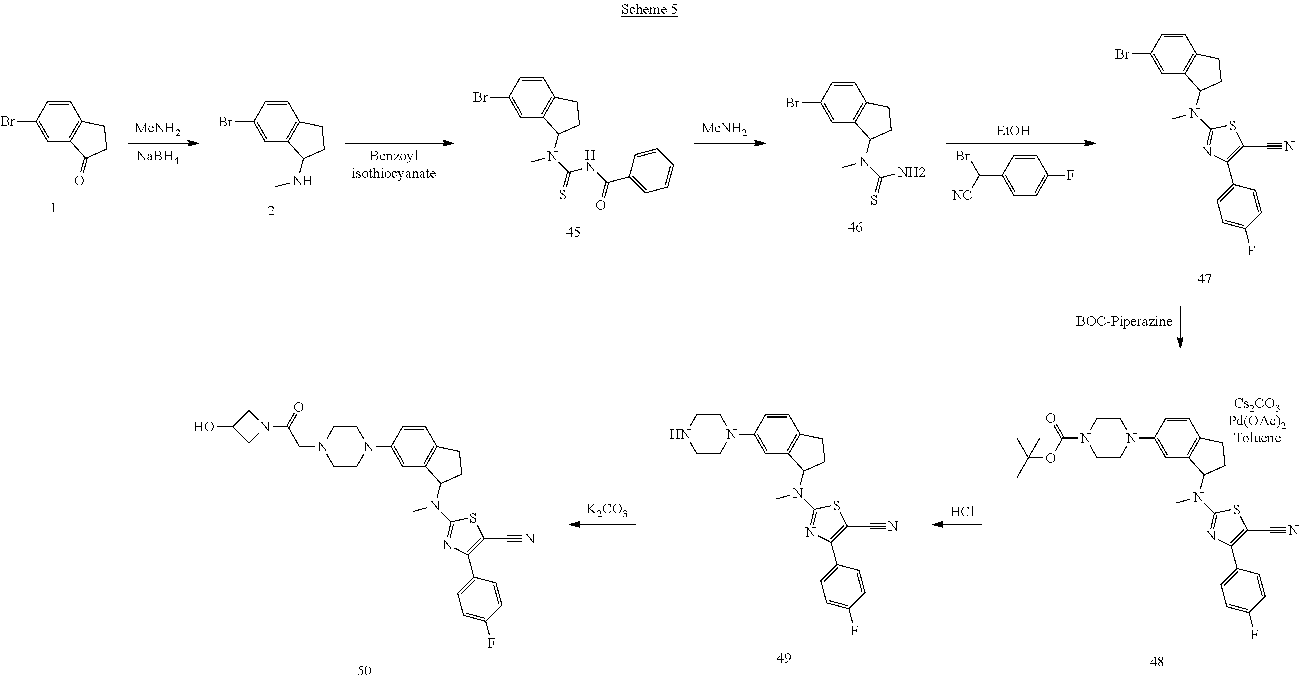

For the preparation of Compounds 50-54, please refer to Scheme 5 and the following details:

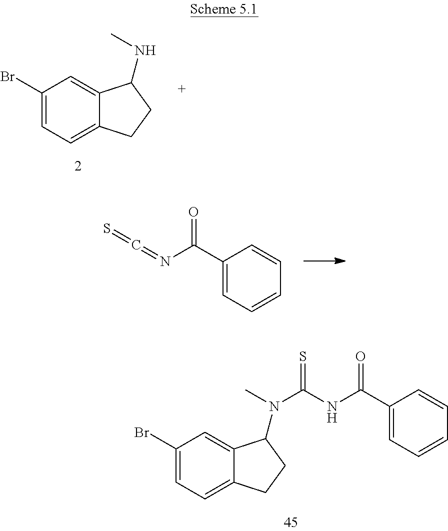 [SMILES: CNC1CCc2ccc(Br)cc21 ; O=C(N=C=S)c1ccccc1 ; CN(C(=S)NC(=O)c1ccccc1)C1CCc2ccc(Br)cc21]

Benzoyl isothiocyanate (0.72 g, 4.42 mmol) was added to a solution of Compound 2 (6-bromo-N-methyl-2,3-dihydro-1H-inden-1-amine) (1.00 g, 4.42 mmol) in ACN (40 mL) and stirred for 3 hours at room temperature to obtain a reaction mixture. After the reaction was completed, the reaction mixture was diluted with cold water and sit to get a precipitated solid. The precipitated solid was collected by filtration and washed with ether to afford a crude product. The crude product was used as Compound 45 in the next step without further purification.

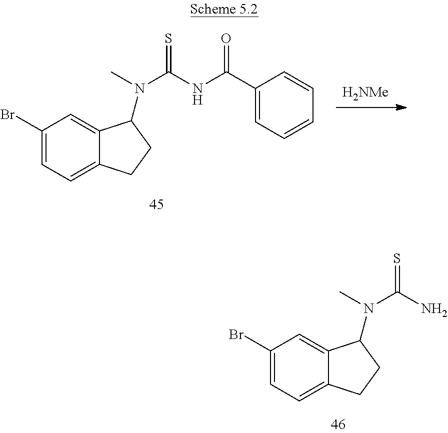 [SMILES: CN(C(=S)NC(=O)c1ccccc1)C1CCc2ccc(Br)cc21 ; CN(C(N)=S)C1CCc2ccc(Br)cc21 ; "H2NMe"]

A solution of Compound 45 (N-((6-bromo-2,3-dihydro-1H-inden-1-yl)(methyl)carbamothioyl) benzamide) (4.0 mmol) in MeOH (20 mL) was added to a solution of methylamine in MeOH (20 mL) and stirred at room temperature overnight to form a mixture. After reaction was completed, the solvent in the mixture was removed under reduced pressure to obtain a crude product. The crude product was used as Compound 46 in the next step without further purification.

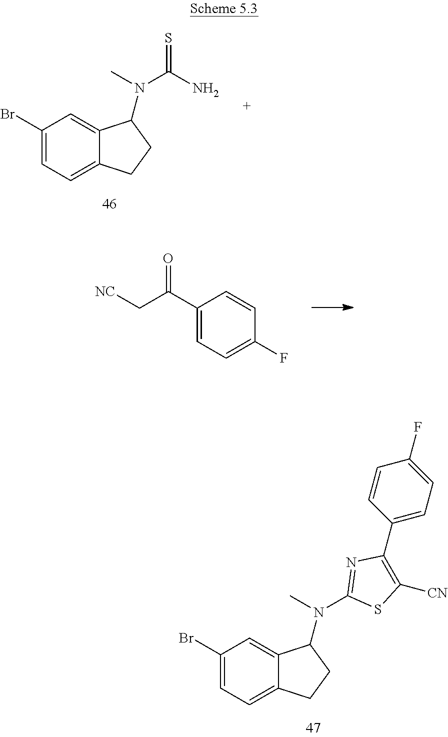 [SMILES: CN(C(N)=S)C1CCc2ccc(Br)cc21 ; N#CCC(=O)c1ccc(F)cc1 ; CN(c1nc(-c2ccc(F)cc2)c(C#N)s1)C1CCc2ccc(Br)cc21]

Pyridine (0.20 g, 2.53 mmol) was added to a solution of 4-fluorobenzoylacetonitrile (0.34 g, 2.10 mmol) in EtOH (10 mL) and heated to 80° C. for 15 minutes to form a reaction mixture. The reaction mixture was cooled to room temperature and a solution of Compound 46 (1-(6-bromo-2,3-dihydro-1H-inden-1-yl)-1-methylthiourea) (0.30 g, 1.05 mmol) and I₂ (0.63 g, 2.50 mmol) in EtOH (10 mL) was added therein. After addition, the reaction mixture was stirred at room temperature overnight. After the reaction was completed, the solvent in the reaction mixture was removed under reduced pressure. The residue was diluted with 1M Na₂S₂O₃ and extracted with EtOAc, and the resulting organic layers were combined. The combined organic layers were dried over MgSO₄ and concentrated in vacuo to obtain a crude product. The crude product was purified via flash column chromatography on a silica gel column using 10:1 DCM-MeOH as the eluent to give Compound 47, 2-((6-bromo-2,3-dihydro-1H-inden-1-yl)(methyl)amino)-4-(4-fluorophenyl)thiazole-5-carbonitrile and the yield thereof was 0.23 g (0.45 mmol).

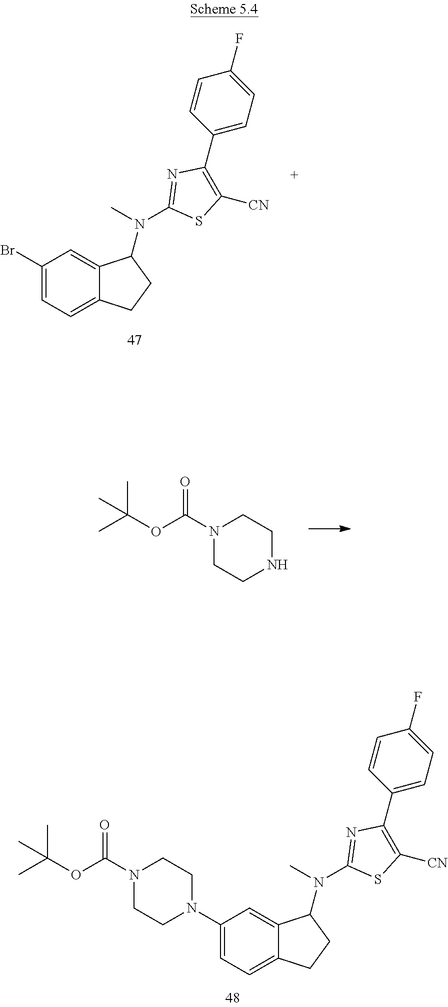 [SMILES: CN(c1nc(-c2ccc(F)cc2)c(C#N)s1)C1CCc2ccc(Br)cc21 ; CC(C)(C)OC(=O)N1CCNCC1 ; CN(c1nc(-c2ccc(F)cc2)c(C#N)s1)C1CCc2ccc(N3CCN(C(=O)OC(C)(C)C)CC3)cc21]

CsCO₃ (0.19 g, 0.60 mmol), Boc-piperazine (0.11 g, 0.60 mmol), 2-(di-t-butylphosphino)biphenyl (0.01 g, 0.04 mmol) and Pd(OAc)₂ (0.01 g, 0.04 mmol) were added to a solution of Compound 47 (2-((6-bromo-2,3-dihydro-1H-inden-1-yl)(methyl)amino)-4-(4-fluorophenyl)thiazole-5-carbonitrile) (0.17 g, 0.40 mmol) in toluene (5 mL) to form a mixture. The mixture was degassed with argon for 15 minutes, and then heated to reflux overnight. After the reaction was completed, the solvent in the mixture was removed under reduced pressure, and then the residue was filtered through celite and washed with EtOAc to obtain a crude product. After concentration in vacuo, the crude product was purified via flash column chromatography on a silica gel column using 10:1 Hexane-EtOAc as the eluent to give Compound 48, tert-butyl 4-(3-((5-cyano-4-(4-fluorophenyl)thiazol-2-yl)(methyl)amino)-2,3-dihydro-1H-inden-5-yl)piperazine-1-carboxylate (0.08 g, 0.16 mmol).

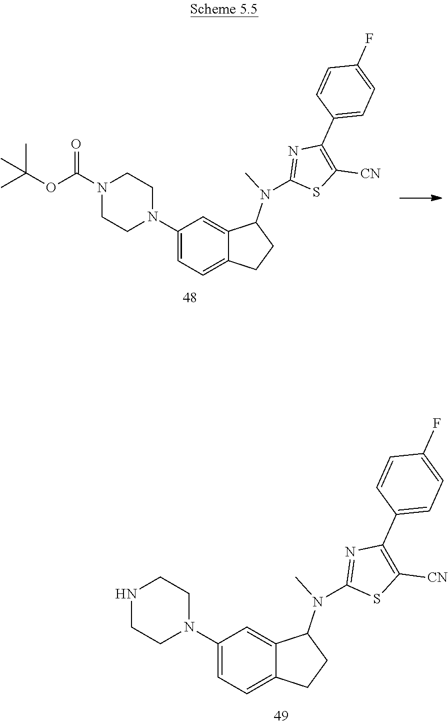 [SMILES: CN(c1nc(-c2ccc(F)cc2)c(C#N)s1)C1CCc2ccc(N3CCN(C(=O)OC(C)(C)C)CC3)cc21 ; CN(c1nc(-c2ccc(F)cc2)c(C#N)s1)C1CCc2ccc(N3CCNCC3)cc21]

Compound 48 (Tert-butyl 4-(3-((5-cyano-4-(4-fluorophenyl)thiazol-2-yl)(methyl)amino)-2,3-dihydro-1H-inden-5-yl)piperazine-1-carboxylate) (0.08 g, 0.16 mmol) was added to a solution of 4N HCl in dioxane (5 mL), and then stirred for 3 hours to form a mixture. After the reaction was completed, the solvent in the mixture was removed under reduced pressure to obtain a crude product. The crude product was used as Compound 49 in the next step without further purification.

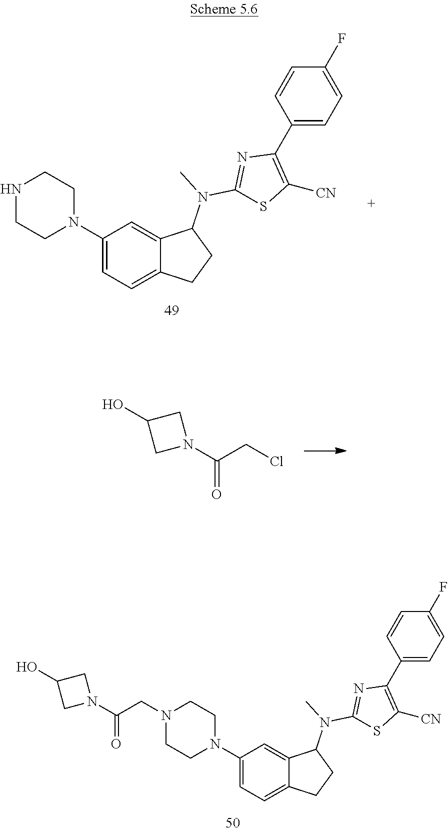 [SMILES: CN(c1nc(-c2ccc(F)cc2)c(C#N)s1)C1CCc2ccc(N3CCNCC3)cc21 ; O=C(CCl)N1CC(O)C1 ; CN(c1nc(-c2ccc(F)cc2)c(C#N)s1)C1CCc2ccc(N3CCN(CC(=O)N4CC(O)C4)CC3)cc21]

K₂CO₃ (0.13 g, 0.94 mmol), 2-chloro-1-(3-hydroxyazetidin-1-yl)ethan-1-one (0.03 g, 0.19 mmol) and a catalytic amount of KI were added to a solution of Compound 49 (4-(4-fluorophenyl)-2-(methyl(6-(piperazin-1-yl)-2,3-dihydro-1H-inden-1-yl)amino)thiazole-5-carbonitrile) in DMF (5 mL), and then the reaction mixture was heated to 80° C. overnight. After the reaction was completed, the solvent in the reaction mixture was evaporated off by air-drying. The residue was diluted with water and extracted with EtOAc, and the resulting organic layers were combined. The combined organic layers were dried over MgSO₄ and concentrated in vacuo to obtain a crude product. The crude product was purified via flash column chromatography on a silica gel column using 10:1 DCM-MeOH as the eluent to give Compound 50, 4-(4-fluorophenyl)-2-((6-(4-(2-(3-hydroxyazetidin-1-yl)-2-oxoethyl)piperazin-1-yl)-2,3-dihydro-1H-inden-1-yl)(methyl)amino)thiazole-5-carbonitrile, and the yield thereof was 0.06 g (0.11 mmol).

Compound 52 was produced by using the same methods for Compound 50.

Compounds 51 and 53 were produced by using the same methods for Compound 50 by replacing Compound 2 with Compound 35 in the corresponding schemes. Compound 54 was produced by using the same methods of Schemes 5.1 to 5.5 and 1.8 with the corresponding starting material.

Spectral Data of Compounds 50-54

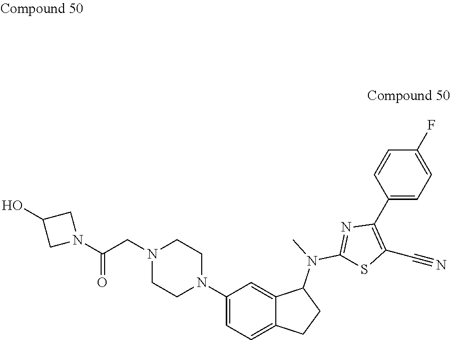 [SMILES: CN(c1nc(-c2ccc(F)cc2)c(C#N)s1)C1CCc2ccc(N3CCN(CC(=O)N4CC(O)C4)CC3)cc21]

Compound 50, ¹H-NMR (400 MHz, CDCl₃): 8.14-8.11 (m, 2H), 7.20-7.12 (m, 3H), 6.91-6.89 (dd, 1H), 6.72 (d, 1H), 5.84 (br, 1H), 4.70-4.67 (m, 1H), 4.48-4.44 (m, 1H), 4.31-4.27 (m, 1H), 4.11-4.08 (m, 1H), 3.91-3.88 (m, 1H), 3.18-3.16 (m, 4H), 3.08 (d, 2H), 2.98-2.84 (m, 5H), 2.67-2.65 (m, 4H), 2.59-2.54 (m, 1H), 2.12 (br, 1H), 2.07-2.02 (m, 1H). ESI-MS m/z calcd for C₂₉H₃₁FN₆O₂S 546.22, found 547.3 [M+H]⁺.

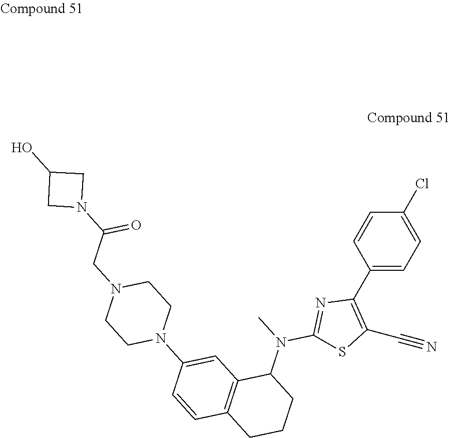 [SMILES: CN(c1nc(-c2ccc(Cl)cc2)c(C#N)s1)C1CCCc2ccc(N3CCN(CC(=O)N4CC(O)C4)CC3)cc21]

Compound 51, ¹H-NMR (400 MHz, CDCl₃): 8.07 (d, 2H), 7.48 (d, 2H), 7.06 (d, 1H), 6.92 (dd, 1H), 6.60 (s, 1H), 4.71-4.63 (m, 1H), 4.47-4.43 (m, 1H), 4.31-4.26 (m, 1H), 4.10-4.08 (m, 1H), 3.92-3.88 (m, 1H), 3.13-3.10 (m, 4H), 3.06 (s, 2H), 2.89 (s, 2H), 2.78-2.72 (m, 2H), 2.64-2.62 (m, 4H), 2.17 (m, 5H), 2.05-2.02 (m, 1H), 1.89-1.84 (m, 1H). ESI-MS m/z calcd for C₃₀H₃₃Cl N₆O₂S 576.21, found 577.3 [M+H]⁺.

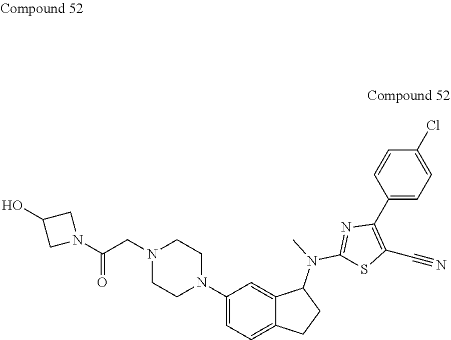 [SMILES: CN(c1nc(-c2ccc(Cl)cc2)c(C#N)s1)C1CCc2ccc(N3CCN(CC(=O)N4CC(O)C4)CC3)cc21]

Compound 52, ¹H-NMR (400 MHz, CDCl₃): 8.08-8.05 (d, 2H), 7.43-7.42 (d, 2H), 7.19-7.17 (d, 1H), 6.90-6.88 (dd, 1H), 6.71 (s, 1H), 5.85 (br, 1H), 4.70-4.67 (m, 1H), 4.48-4.44 (m, 1H), 4.31-4.27 (m, 1H), 4.13-4.09 (m, 1H), 3.91-3.87 (m, 1H), 3.17-3.16 (m, 4H), 3.08 (d, 2H), 3.02-2.82 (m, 5H), 2.67-2.64 (m, 4H), 2.60-2.53 (m, 1H), 2.1 (br, 1H), 2.09-1.99 (m, 1H). ESI-MS m/z calcd for C₂₉H₃₁ClN₆O₂S 562.19, found 563.3 [M+H]⁺.

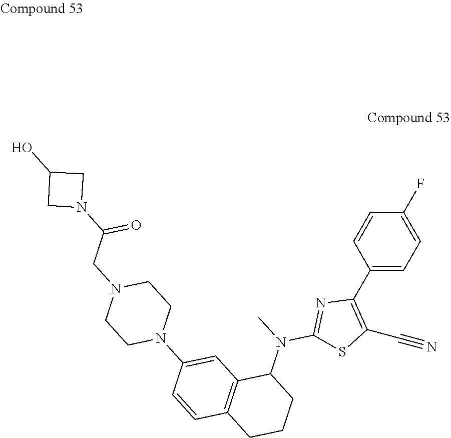 [SMILES: CN(c1nc(-c2ccc(F)cc2)c(C#N)s1)C1CCCc2ccc(N3CCN(CC(=O)N4CC(O)C4)CC3)cc21]

Compound 53, ¹H-NMR (400 MHz, CDCl₃): 8.12 (dd, 2H), 7.16-7.12 (m, 2H), 7.06 (d, 1H), 6.83 (dd, 1H), 6.60 (d, 1H), 4.70-4.61 (m, 1H), 4.44 (dd, 1H), 4.28 (dd, 1H), 4.11-4.07 (m, 1H), 3.88 (dd, 1H), 3.60 (br, 1H), 3.13-3.10 (m, 4H), 3.06 (s, 2H), 2.88 (s, 3H), 2.78-2.74 (m, 2H), 2.64-2.62 (m, 4H), 2.19-2.16 (m, 2H), 2.05-1.99 (m, 1H), 1.89-1.84 (m, 2H). ESI-MS m/z calcd for C₃₀H₃₃FN₆O₂S 560.24, found 561.3 [M+H]⁺.

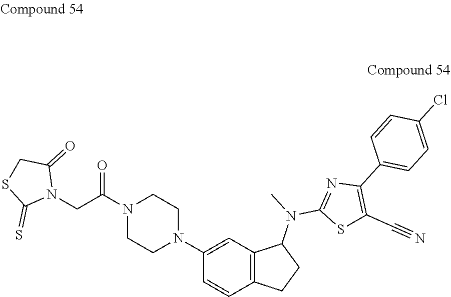 [SMILES: CN(c1nc(-c2ccc(Cl)cc2)c(C#N)s1)C1CCc2ccc(N3CCN(C(=O)CN4C(=O)CSC4=S)CC3)cc21]

Compound 54, 1H-NMR (400 MHz, CDCl₃): 8.08-8.06 (m, 2H), 7.45-7.42 (m, 2H), 7.23-7.21 (m, 1H), 6.92-6.90 (m, 1H), 6.73 (s, 1H), 5.84 (br, 1H), 4.84 (s, 2H), 4.09 (s, 2H), 3.76-3.68 (m, 4H), 3.22-3.13 (m, 4H), 3.05-2.86 (m, 5H), 2.62-2.54 (m, 1H), 2.17-2.02 (m, 1H). ESI-MS m/z calcd for C₂₉H₂₇ClN₆O₂S₃ 622.10, found 623.2 [M+H]⁺

### Example 7. Compounds 58-61

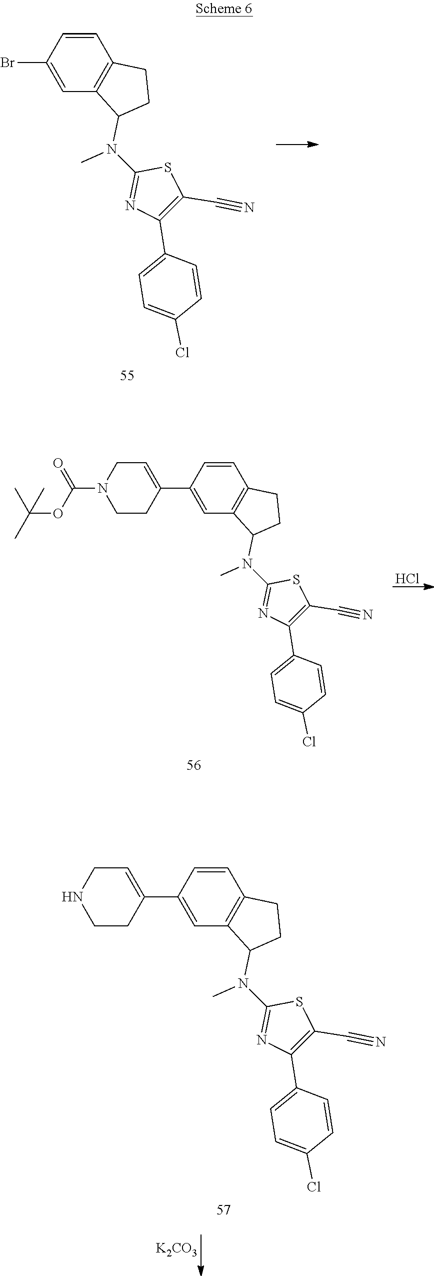

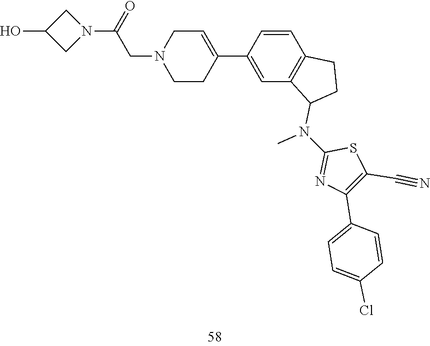 [SMILES: CN(c1nc(-c2ccc(Cl)cc2)c(C#N)s1)C1CCc2ccc(C3=CCN(CC(=O)N4CC(O)C4)CC3)cc21]

For the preparation of Compounds 58-61, please refer to Scheme 6 and the following details:

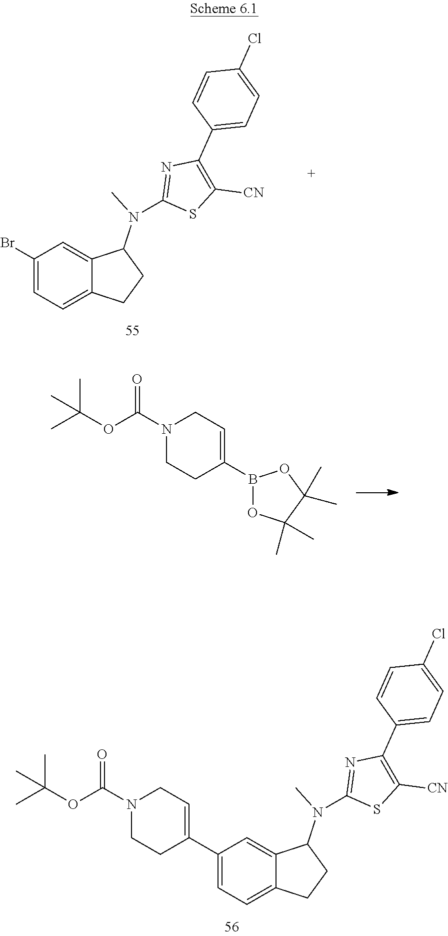

Compound 55 was produced with the same methods for Compound 47 by replacing 4-fluorobenzoylacetonitrile with 4-chlorobenzoylacetonitrile. Na₂CO₃ (0.08 g, 0.77 mmol), N-Boc-1,2,3,6-tetrahydropyridine-4-boronic acid pinacol ester (0.16 g, 0.52 mmol), and Pd(dppf)Cl₂ (0.01 g, 0.01 mmol) were added to a solution of Compound 55 (2-((6-bromo-2,3-dihydro-1H-inden-1-yl)(methyl)amino)-4-(4-chlorophenyl)thiazole-5-carbonitrile) (0.12 g, 0.26 mmol) in DMF (5 mL) to form a mixture. The mixture was degassed with argon for 15 minutes, and then heated to 100° C. overnight. After the reaction was completed, the solvent in the mixture was evaporated off by air-drying, and then the residue was filtered through celite and washed with EtOAc to obtain a crude product. After concentration in vacuo, the crude product was purified via flash column chromatography on a silica gel column using 10:1 Hexane-EtOAc as the eluent to give Compound 56, tert-butyl 4-(3-((4-(4-chlorophenyl)-5-cyanothiazol-2-yl)(methyl)amino)-2,3-dihydro-1H-inden-5-yl)-3,6-dihydropyridine-1(2H)-carboxylate (0.14 g, 0.25 mmol).

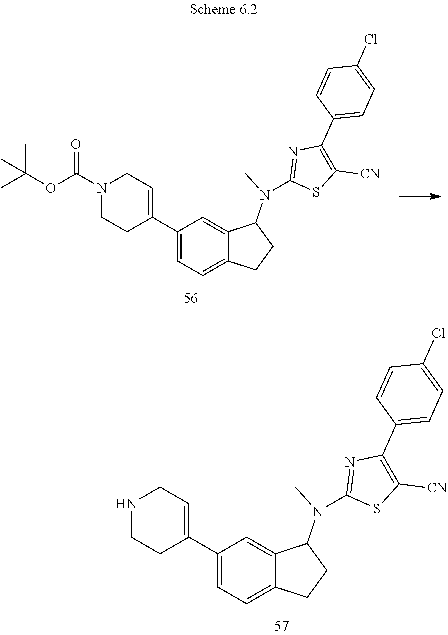 [SMILES: CN(c1nc(-c2ccc(Cl)cc2)c(C#N)s1)C1CCc2ccc(C3=CCN(C(=O)OC(C)(C)C)CC3)cc21 ; CN(c1nc(-c2ccc(Cl)cc2)c(C#N)s1)C1CCc2ccc(C3=CCNCC3)cc21]

Compound 56 (tert-butyl 4-(3-((4-(4-chlorophenyl)-5-cyanothiazol-2-yl)(methyl)amino)-2,3-dihydro-1H-inden-5-yl)-3,6-dihydropyridine-1(2H)-carboxylate) (0.14 g, 0.25 mmol) was added to a solution of 4N HCl in dioxane (5 mL), and then stirred for 3 hours to form a mixture. After the reaction was completed, the solvent in the mixture was removed under reduced pressure to obtain a crude product. The crude product was used as Compound 57 in the next step without further purification.

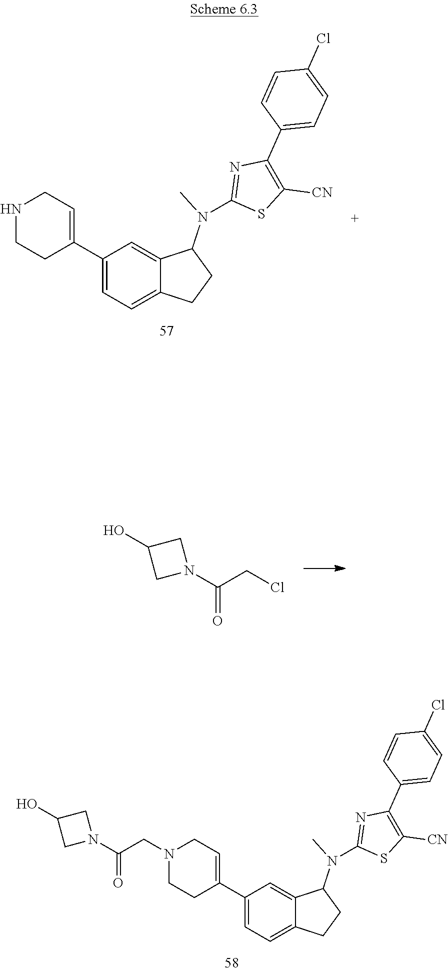 [SMILES: CN(c1nc(-c2ccc(Cl)cc2)c(C#N)s1)C1CCc2ccc(C3=CCNCC3)cc21 ; O=C(CCl)N1CC(O)C1 ; CN(c1nc(-c2ccc(Cl)cc2)c(C#N)s1)C1CCc2ccc(C3=CCN(CC(=O)N4CC(O)C4)CC3)cc21]

K₂CO₃ (0.36 g, 2.57 mmol), 2-chloro-1-(3-hydroxyazetidin-1-yl)ethan-1-one (0.05 g, 0.31 mmol) and a catalytic amount of KI were added to a solution of Compound 57 (4-(4-chlorophenyl)-2-(methyl(6-(1,2,3,6-tetrahydropyridin-4-yl)-2,3-dihydro-1H-inden-1-yl)amino)thiazole-5-carbonitrile) in DMF (5 mL), and then the reaction mixture was heated at 80° C. overnight. After the reaction was completed, the solvent in the mixture was evaporated off by air-drying. The residue was diluted with water and extracted with EtOAc, and the resulting organic layers are combined. The combined organic layers were dried over MgSO₄ and concentrated in vacuo to obtain a crude product. The crude product was purified via flash column chromatography on a silica gel column using 10:1 DCM-MeOH as the eluent to give Compound 58, 4-(4-chlorophenyl)-2-((6-(1-(2-(3-hydroxyazetidin-1-yl)-2-oxoethyl)-1,2,3,6-tetrahydropyridin-4-yl)-2,3-dihydro-1H-inden-1-yl)(methyl)amino)thiazole-5-carbonitrile, and the yield thereof was 0.06 g (0.11 mmol).

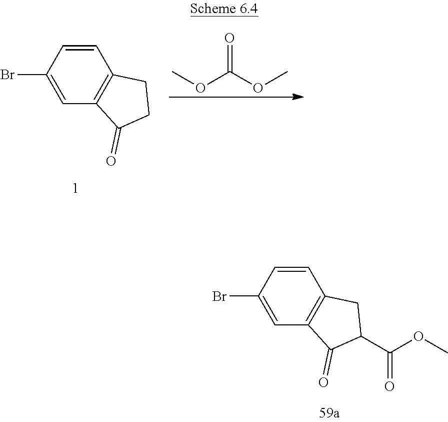

To a stirred suspension of NaH (0.86 g, 60% in mineral oil, 20 mmol) in 20 mL dimethyl carbonate was added dropwise a solution of Compound 1 (6-bromoindanone) (1.0 g, 4.73 mmol) in 30 mL dimethyl carbonate and DMF 1 mL. The mixture was refluxed at 80° C. for 16 hours. After cooling to room temperature, and then H₂O (80 mL) was added. The aqueous phase was separated and extracted with CH₂Cl₂ (3×50 mL). The combined organic extracts were dried (MgSO₄) and concentrated under reduced pressure. The crude oil thus obtained was subjected to chromatography (silica gel, 2:1 hexane/CH₂Cl₂) to yield 1.06 g (83%) of Compound 59a (methyl 6-bromo-1-oxo-2,3-dihydro-1H-indene-2-carboxylate).

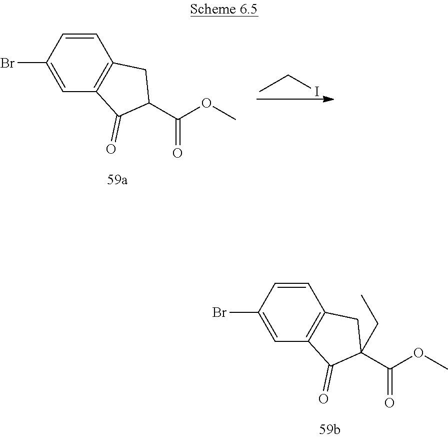

A solution of Compound 59a (methyl 6-bromo-1-oxo-2,3-dihydro-1H-indene-2-carboxylate) (0.27 g, 1.0 mmol) in dry DMSO (5 ml) was stirred under argon and cooled in a water bath. K₂CO₃ (0.28 g, 2.0 mmol) was added and the resulting suspension stirred for a further 15 minutes, lodoethane (0.31 g, 2.0 mmol) was added and the mixture was stirred for 16 hours at rt. Ethyl acetate and excess dilute aqueous hydrochloric acid were added. The organic layer was separated, dried and evaporated to give a brown solid (Compound 59b) (0.25 g, 84%). The crude product of Compound 59b was used in next step without further purification.

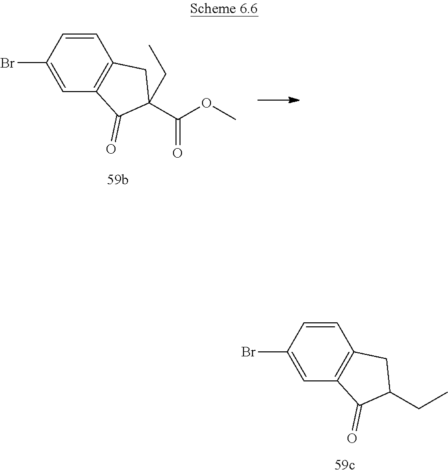 [SMILES: CCC1(C(=O)OC)Cc2ccc(Br)cc2C1=O ; CCC1Cc2ccc(Br)cc2C1=O]

To a solution of Compound 59b (methyl 6-bromo-2-ethyl-1-oxo-2,3-dihydro-1H-indene-2-carboxylate) (3.70 g, 14.46 mmol) in AcOH (30 mL) was added H₂O (2 mL) and conc. HCl (10 mL). After addition, the reaction mixture was refluxed for overnight. After reaction was completed, the solvent AcOH was removed under reduced pressure. The residual was dissolved in EtOAc and washed with Sat. NaHCO₃ and brine. The combined organic layers were dried over MgSO₄ and concentrated in vacuo to get a crude product of Compound 59c (2.71 g, 11.33 mmol). The Compound 59c was used in the next step without further purification.

Compound 59 was produced by using the same methods for Compound 58 by replacing with the corresponding starting materials. Compounds 60 and 61 were produced by using the same methods for Compounds 58 and 59, respectively, by replacing Compound 1 with Compound 59c in the corresponding schemes.

Spectral Data of Compounds 58-61

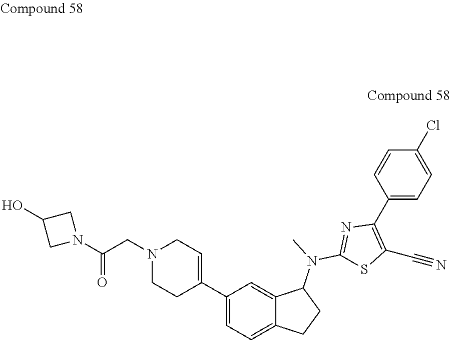 [SMILES: CN(c1nc(-c2ccc(Cl)cc2)c(C#N)s1)C1CCc2ccc(C3=CCN(CC(=O)N4CC(O)C4)CC3)cc21]

Compound 58, ¹H-NMR (300 MHz, CDCl₃): 8.08-8.05 (d, 2H), 7.44-7.41 (d, 2H), 7.34-7.18 (m, 3H), 6.02 (s, 1H), 5.90 (br, 1H), 4.68-4.64 (m, 1H), 4.50-4.44 (m, 1H), 4.32-4.26 (m, 1H), 4.13-4.09 (m, 1H), 3.92-3.87 (m, 1H), 3.21-3.20 (m, 2H), 3.16 (s, 2H), 3.10-2.94 (m, 2H), 2.89 (s, 3H), 2.78-2.74 (t, 2H), 2.65-2.55 (m, 3H), 2.17-2.00 (m, 2H). ESI-MS m/z calcd for C₃₀H₃₀ClN₅O₂S 559.18, found 560.3 [M+H]⁺.

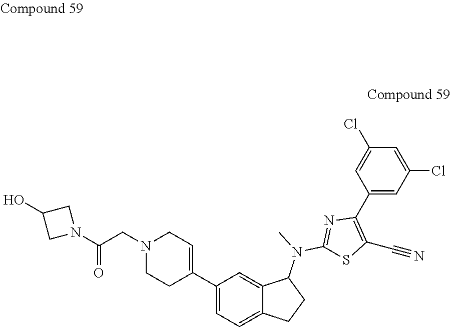 [SMILES: CN(c1nc(-c2cc(Cl)cc(Cl)c2)c(C#N)s1)C1CCc2ccc(C3=CCN(CC(=O)N4CC(O)C4)CC3)cc21]

Compound 59, ¹H-NMR (300 MHz, CDCl₃): 7.99-7.98 (t, 2H), 7.54-7.53 (m, 1H), 7.40-7.24 (m, 3H), 6.06 (s, 1H), 5.95 (br, 1H), 4.61-4.54 (m, 1H), 4.50-4.44 (m, 1H), 4.24-4.18 (m, 1H), 4.06-4.02 (m, 1H), 3.79-3.75 (m, 1H), 3.24-3.21 (m, 4H), 3.08-2.98 (m, 2H), 2.91 (s, 3H), 2.82 (t, 2H), 2.61-2.57 (m, 3H), 2.25-2.10 (m, 2H). ESI-MS m/z calcd for C₃₀H₂₉Cl₂N₅O₂S 593.14, found 594.3 [M+H]⁺.

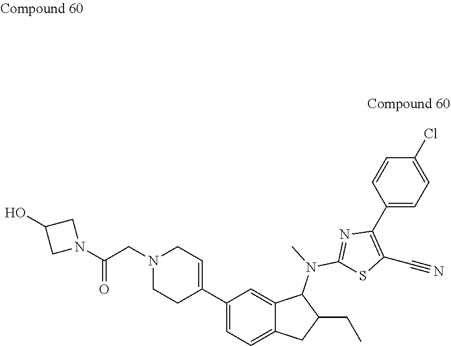 [SMILES: CCC1Cc2ccc(C3=CCN(CC(=O)N4CC(O)C4)CC3)cc2C1N(C)c1nc(-c2ccc(Cl)cc2)c(C#N)s1]

Compound 60, ¹H-NMR (400 MHz, CDCl₃): 8.09-8.06 (m, 2H), 7.44-7.41 (m, 2H), 7.37-7.35 (m, 1H), 7.31-7.22 (m, 2H), 6.03 (s, 1H), 4.68-4.65 (m, 1H), 4.48-4.44 (m, 1H), 4.31-4.27 (m, 1H), 4.15-4.10 (m, 1H), 3.91-3.88 (m, 1H), 3.21-3.16 (m, 4H), 2.76-2.59 (m, 7H), 2.23-2.21 (m, 1H), 1.28-1.21 (m, 6H). 1.01 (t, 3H). ESI-MS m/z calcd for C₃₂H₃₄ClN₅O₂S 587.21, found 588.3 [M+H]⁺.

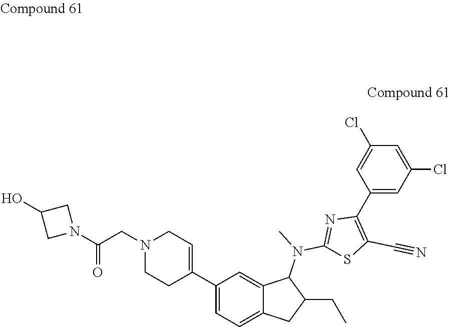 [SMILES: CCC1Cc2ccc(C3=CCN(CC(=O)N4CC(O)C4)CC3)cc2C1N(C)c1nc(-c2cc(Cl)cc(Cl)c2)c(C#N)s1]

Compound 61, ¹H-NMR (300 MHz, CDCl₃): 8.00 (s, 2H), 7.42 (s, 2H), 7.31-7.21 (m, 2H), 6.03 (s, 1H), 5.99 (br, 1H), 4.66-4.62 (m, 1H), 4.48-4.43 (m, 1H), 4.31-4.25 (m, 1H), 4.13-4.09 (m, 1H), 3.92-3.87 (m, 1H), 3.21-3.11 (m, 6H), 2.76-2.55 (m, 9H), 1.63-1.55 (m, 2H), 1.25-1.24 (t, 3H). ESI-MS m/z calcd for C₃₂H₃₃Cl₂N₅O₂S 621.17, found 622.3 [M+H]⁺.

### Example 8. Compound 66

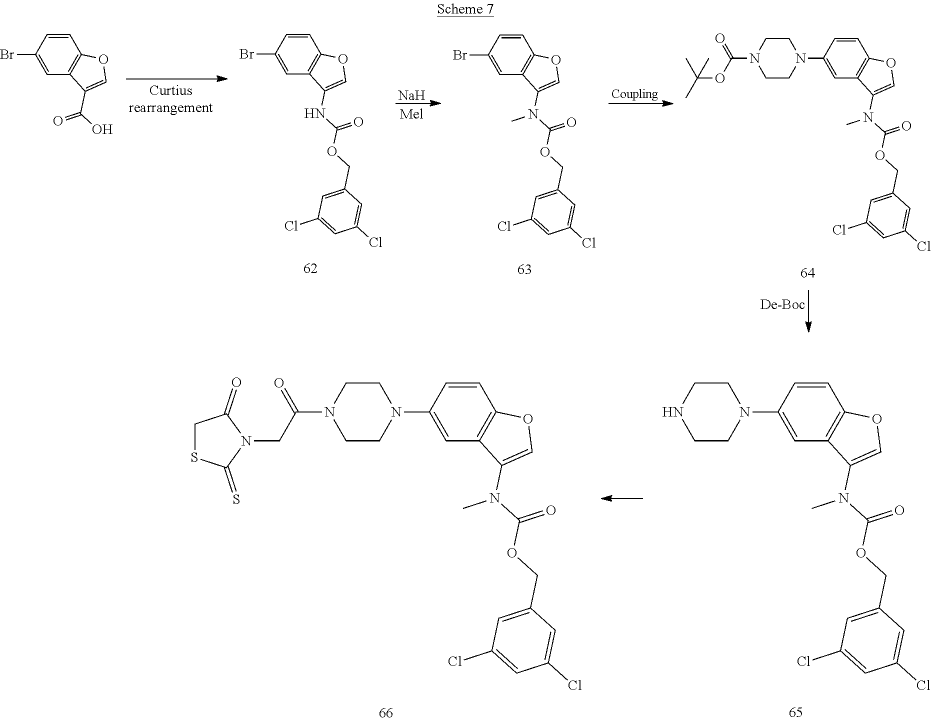

For the preparation of Compound 66, please refer to Scheme 7 and the following details:

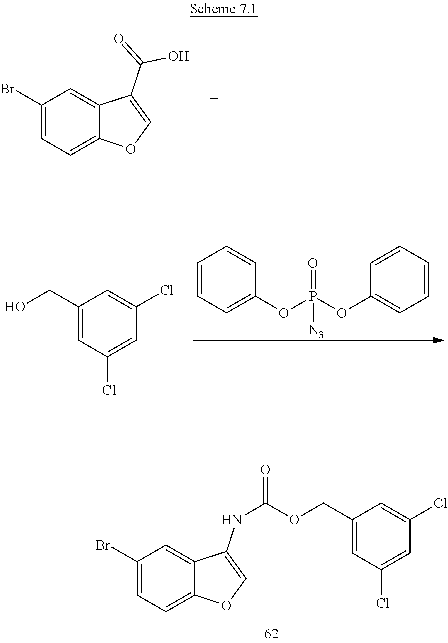

TEA (0.42 g, 4.13 mmol) and diphenylphosphorylazide (0.68 g, 2.48 mmol) were added to a solution of 5-bromobenzofuran-3-carboxylic acid (0.50 g, 2.06 mmol) and 3,5-dichlorobenzylalcohol (0.43 g, 2.48 mmol) in toluene (20 mL). After addition, the reaction mixture was heated to reflux overnight. After the reaction was completed, the solvent was removed under reduced pressure. The residue was diluted with EtOAc and washed with saturated NH₄Cl, saturated NaHCO₃ and brine, and the resulting organic layers were combined. The combined organic layers were dried over MgSO₄ and concentrated in vacuo. The crude product was purified via flash column chromatography on a silica gel column using 10:1 Hexane-EtOAc as the eluent to give Compound 62, 3,5-dichlorobenzyl (5-bromobenzofuran-3-yl)carbamate (0.53 g, 1.28 mmol).

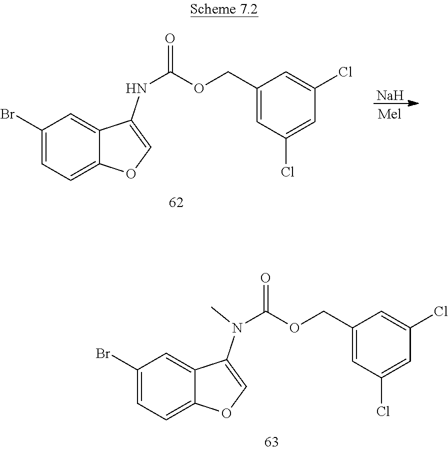

NaH (0.07 g) was added to a solution of Compound 62 (3,5-dichlorobenzyl (5-bromobenzofuran-3-yl)carbamate) (0.53 g, 1.28 mmol) in MeCN (20 mL) at 0° C. and stirred for 30 minutes at the same temperature to form a reaction mixture, and then MeI (1 mL) was added into the reaction mixture. After addition, the reaction mixture was slowly warmed to room temperature and stirred for 1 hour. After the reaction was completed, the solvent in the reaction mixture was removed under reduced pressure. The residue was diluted with EtOAc and washed with saturated NH₄Cl, saturated NaHCO₃ and brine, and the resulting organic layers are combined. The combined organic layers were dried over MgSO₄ and concentrated in vacuo to obtain a crude product. The crude product was purified via flash column chromatography on a silica gel column using 10:3 Hexane-EtOAc as the eluent to give Compound 63, 3,5-dichlorobenzyl (5-bromobenzofuran-3-yl)(methyl)carbamate (0.44 g, 1.03 mmol).

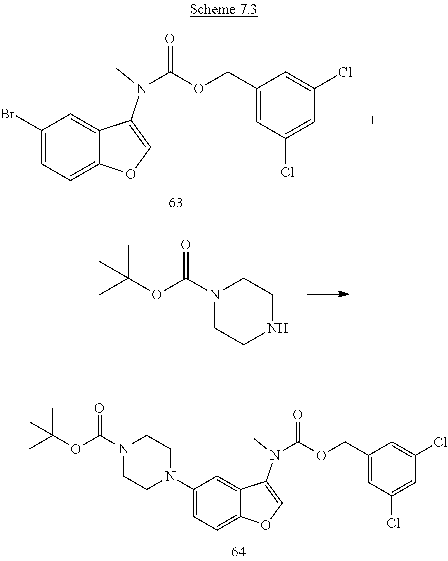 [SMILES: CN(C(=O)OCc1cc(Cl)cc(Cl)c1)c1coc2ccc(Br)cc12 ; CC(C)(C)OC(=O)N1CCNCC1 ; CN(C(=O)OCc1cc(Cl)cc(Cl)c1)c1coc2ccc(N3CCN(C(=O)OC(C)(C)C)CC3)cc12]

CsCO₃ (0.50 g, 1.54 mmol), Boc-piperazine (0.29 g, 1.54 mmol), 2-(di-t-butylphosphino)biphenyl (0.03 g, 0.10 mmol) and Pd(OAc)₂ (0.02 g, 0.01 mmol) were added to a solution of Compound 63 (3,5-dichlorobenzyl (5-bromobenzofuran-3-yl)(methyl)carbamate) (0.44 g, 1.03 mmol) in toluene (10 mL) to form a mixture. The mixture was degassed with argon for 15 minutes, and then heated to reflux overnight. After the reaction was completed, the solvent in the mixture was removed under reduced pressure, and then the residue was filtered through celite and washed with EtOAc to obtain a crude product. After concentration in vacuo, the crude product was purified via flash column chromatography on a silica gel column using 10:3 Hexane-EtOAc as the eluent to give Compound 64, tert-butyl 4-(3-((((3,5-dichlorobenzyl)oxy)carbonyl)(methyl)amino)benzofuran-5-yl)piperazine-1-carboxylate, and the yield thereof was 0.23 g (0.43 mmol).

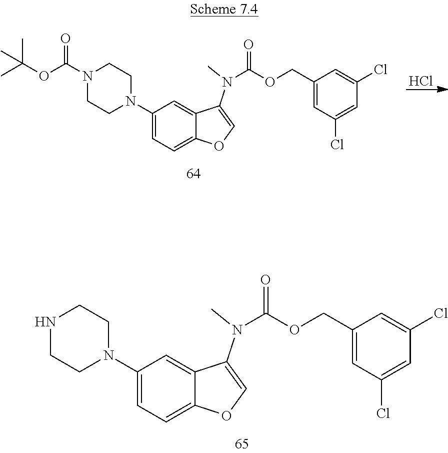 [SMILES: CN(C(=O)OCc1cc(Cl)cc(Cl)c1)c1coc2ccc(N3CCN(C(=O)OC(C)(C)C)CC3)cc12 ; Cl ; CN(C(=O)OCc1cc(Cl)cc(Cl)c1)c1coc2ccc(N3CCNCC3)cc12]

Compound 64 (tert-butyl 4-(3-((((3,5-dichlorobenzyl)oxy)carbonyl)(methyl)amino) benzofuran-5-yl)piperazine-1-carboxylate) (0.23 g, 0.43 mmol) was added to a solution of 4N HCl in dioxane (10 mL), and then stirred for 3 hours to form a mixture. After the reaction was completed, the solvent in the mixture was removed under reduced pressure to produce a crude product. The crude product was used as Compound 65 in the next step without further purification.

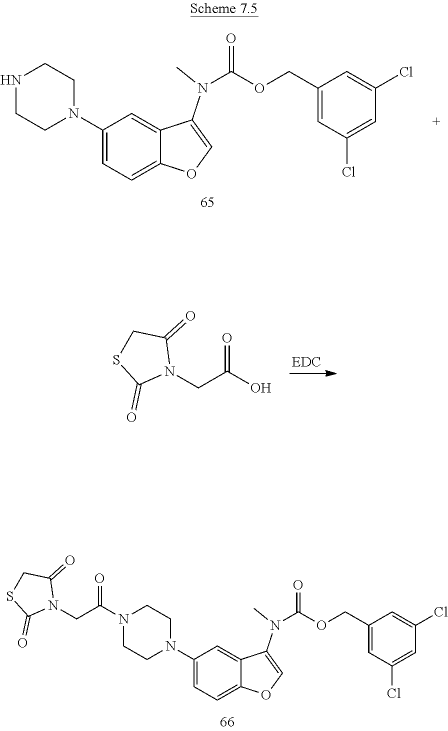 [SMILES: CN(C(=O)OCc1cc(Cl)cc(Cl)c1)c1coc2ccc(N3CCNCC3)cc12 ; O=C(O)CN1C(=O)CSC1=O ; CN(C(=O)OCc1cc(Cl)cc(Cl)c1)c1coc2ccc(N3CCN(C(=O)CN4C(=O)CSC4=O)CC3)cc12]

NMM (0.17 g, 1.70 mmol) and EDCI (0.12 g, 0.64 mmol) were added to a mixture of Compound 65 (3,5-dichlorobenzyl methyl(5-(piperazin-1-yl)benzofuran-3-yl)carbamate) (0.4 mmol), Rhodanine-3-acetic acid (0.12 g, 0.64 mmol), HOBt (0.01 g, 0.09 mmol) in DCM (10 mL) at 0° C. After addition, the reaction mixture was slowly warmed to room temperature and stirred overnight. After the reaction was completed, the solvent in the in the reaction mixture was removed under reduced pressure. The residue was diluted with EtOAc and washed with saturated NH₄Cl, saturated NaHCO₃ and brine, and the resulting organic layers were combined. The combined organic layers were dried over MgSO₄ and concentrated in vacuo to obtain a crude product. The crude product was purified via flash column chromatography on a silica gel column using 10:2 DCM-EtOAc as the eluent to give Compound 66, 3,5-dichlorobenzyl methyl(5-(4-(2-(4-oxo-2-thioxothiazolidin-3-yl)acetyl)piperazin-1-yl)benzofuran-3-yl)carbamate (0.17 g, 0.28 mmol).

Spectral Data of Compound 66

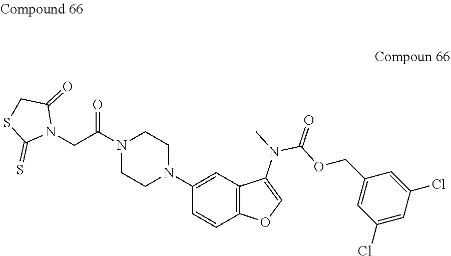 [SMILES: CN(C(=O)OCc1cc(Cl)cc(Cl)c1)c1coc2ccc(N3CCN(C(=O)CN4C(=O)CSC4=S)CC3)cc12]

Compound 66, ¹H-NMR (300 MHz, CDCl₃): 7.63 (s, 1H), 7.43-7.40 (d, 1H), 7.29-7.25 (m, 2H), 7.05-7.01 (m, 2H), 6.87 (br, 1H), 5.10 (br, 2H), 4.87 (s, 2H), 4.10 (s, 2H), 3.78-3.70 (m, 4H), 3.38 (s, 3H), 3.16-3.09 (m, 4H). ESI-MS m/z calcd for C₂₆H₂₄Cl₂N₄O₅S₂ 606.06, found 607.1 [M+H]⁺

### Example 9. Compound 69

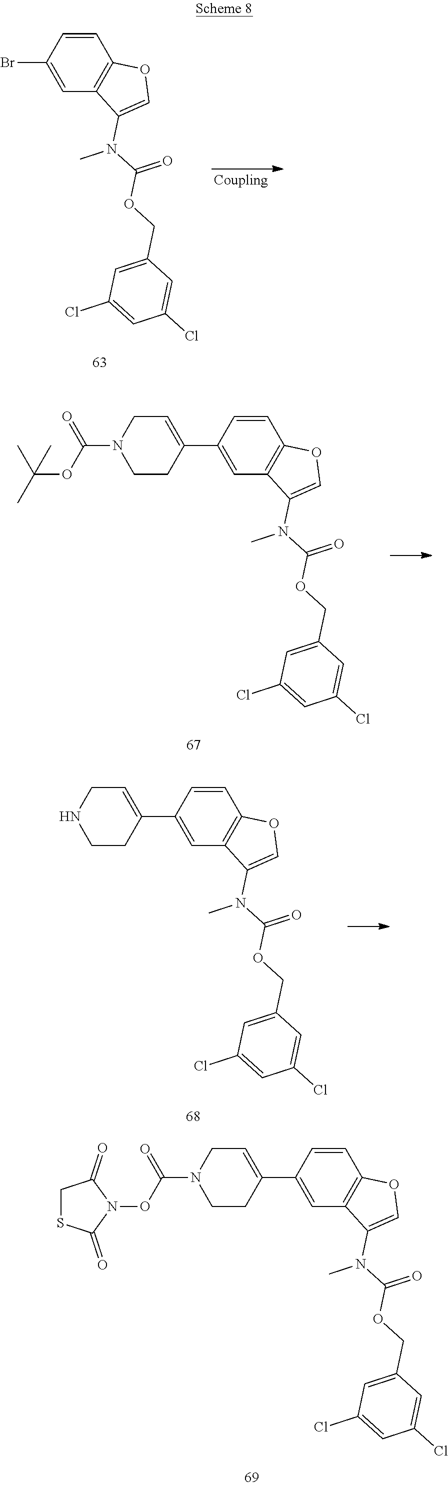 [SMILES: CN(C(=O)OCc1cc(Cl)cc(Cl)c1)c1coc2ccc(Br)cc12 ; CN(C(=O)OCc1cc(Cl)cc(Cl)c1)c1coc2ccc(C3=CCN(C(=O)OC(C)(C)C)CC3)cc12 ; CN(C(=O)OCc1cc(Cl)cc(Cl)c1)c1coc2ccc(C3=CCNCC3)cc12 ; CN(C(=O)OCc1cc(Cl)cc(Cl)c1)c1coc2ccc(C3=CCN(C(=O)ON4C(=O)CSC4=O)CC3)cc12]

For the preparation of Compound 69, please refer to Scheme 8 and the following details:

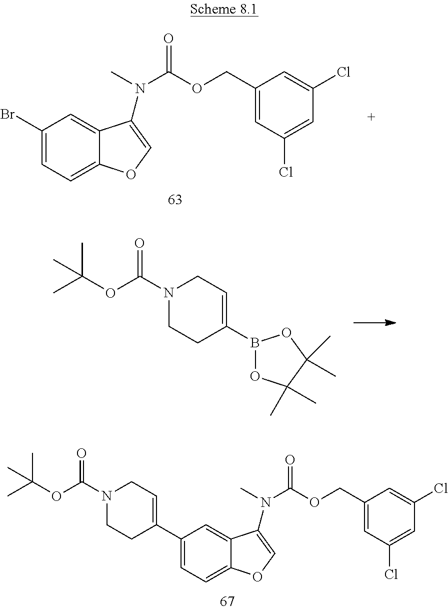

Na₂CO₃ (0.07 g, 0.69 mmol), N-Boc-1,2,3,6-tetrahydropyridine-4-boronic acid pinacol ester (0.14 g, 0.46 mmol), and Pd(dppf)Cl₂ (0.02 g, 0.02 mmol) were added to a solution of Compound 63 (3,5-dichlorobenzyl (5-bromobenzofuran-3-yl)(methyl)carbamate) (0.10 g) in DMF (10 mL) to form a mixture. The mixture was degassed with argon for 15 minutes, and then heated at 100° C. overnight. After the reaction was completed, the solvent in the mixture was evaporated off by air-drying, and then the residue was filtered through celite and washed with EtOAc to obtain a crude product. After concentration in vacuo, the crude product was purified via flash column chromatography on a silica gel column using 10:1 Hexane-EtOAc as the eluent to give Compound 67, tert-butyl 4-(3-((((3,5-dichlorobenzyl)oxy)carbonyl)(methyl)amino)benzofuran-5-yl)-3,6-dihydropyridine-1(2H)-carboxylate (0.07 g, 0.14 mmol).

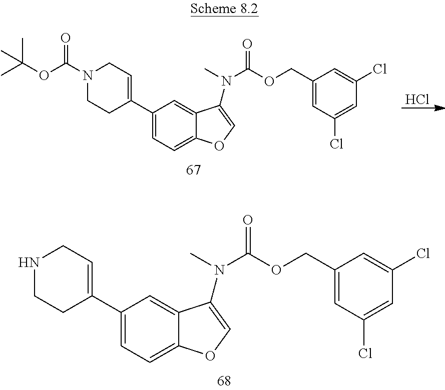 [SMILES: CN(C(=O)OCc1cc(Cl)cc(Cl)c1)c1coc2ccc(C3=CCN(C(=O)OC(C)(C)C)CC3)cc12 ; Cl ; CN(C(=O)OCc1cc(Cl)cc(Cl)c1)c1coc2ccc(C3=CCNCC3)cc12]

Compound 67 (tert-butyl 4-(3-((((3,5-dichlorobenzyl)oxy)carbonyl)(methyl)amino) benzofuran-5-yl)-3,6-dihydropyridine-1(2H)-carboxylate) (0.07 g, 0.14 mmol) was added to a solution of 4N HCl in dioxane (10 mL), and then stirred for 3 hours to form a mixture. After the reaction was completed, the solvent in the mixture was removed under reduced pressure to obtain a crude product. The crude product was used as Compound 68 in the next step without further purification.

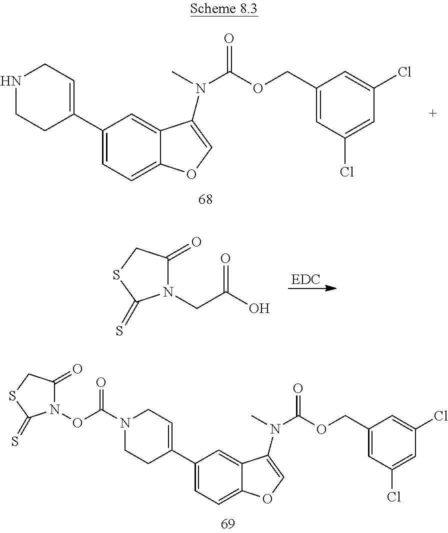 [SMILES: CN(C(=O)OCc1cc(Cl)cc(Cl)c1)c1coc2ccc(C3=CCNCC3)cc12 ; O=C(O)CN1C(=O)CSC1=S ; "EDC" ; CN(C(=O)OCc1cc(Cl)cc(Cl)c1)c1coc2ccc(C3=CCN(C(=O)ON4C(=O)CSC4=S)CC3)cc12]

NMM (0.04 g, 0.41 mmol) and EDCI (0.04 g, 0.20 mmol) were added to a mixture of Compound 68 (3,5-dichlorobenzyl methyl(5-(1,2,3,6-tetrahydropyridin-4-yl)benzofuran-3-yl)carbamate) (0.14 mmol), Rhodanine-3-acetic Acid (0.04 g, 0.20 mmol), HOBt (4.0 mg, 0.03 mmol) in DCM (10 mL) at 0° C. After addition, the reaction mixture was slowly warmed to room temperature and stirred overnight. After the reaction was completed, the solvent in the reaction mixture was removed under reduced pressure. The residue was diluted with EtOAc and washed with saturated NH₄Cl, saturated NaHCO₃ and brine, and the resulting organic layers were combined. The combined organic layers were dried over MgSO₄ and concentrated in vacuo to obtain a crude product. The crude product was purified via flash column chromatography on a silica gel column using 10:2 DCM-EtOAc as the eluent to give Compound 69, 3,5-dichlorobenzyl methyl(5-(1-(2-(4-oxo-2-thioxothiazolidin-3-yl)acetyl)-1,2,3,6-tetrahydropyridin-4-yl)benzofuran-3-yl)carbamate, and the yield thereof was 0.03 g (0.05 mmol).

Spectral Data of Compound 69

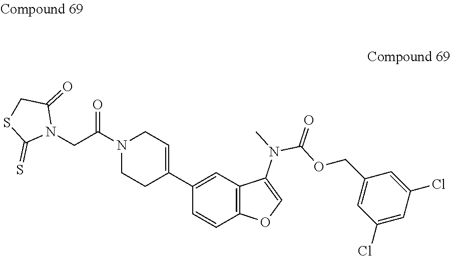 [SMILES: CN(C(=O)OCc1cc(Cl)cc(Cl)c1)c1coc2ccc(C3=CCN(C(=O)CN4C(=O)CSC4=S)CC3)cc12]

Compound 69, ¹H-NMR (300 MHz, CDCl₃): 7.67 (s, 1H), 7.54-7.26 (m, 5H), 7.05 (br, 1H), 6.00-5.94 (m, 1H), 5.09 (br, 2H), 4.91 (s, 1H), 4.86 (s, 1H), 4.24 (s, 2H), 4.10 (s, 2H), 3.85-3.73 (m, 2H), 3.40 (s, 3H), 2.67-2.56 (m, 2H). ESI-MS m/z calcd for C₂₇H₂₃Cl₂N₃O₄S₂ 603.05, found 626.1[M+Na]⁺.

### Example 10. Compounds 73 and 74

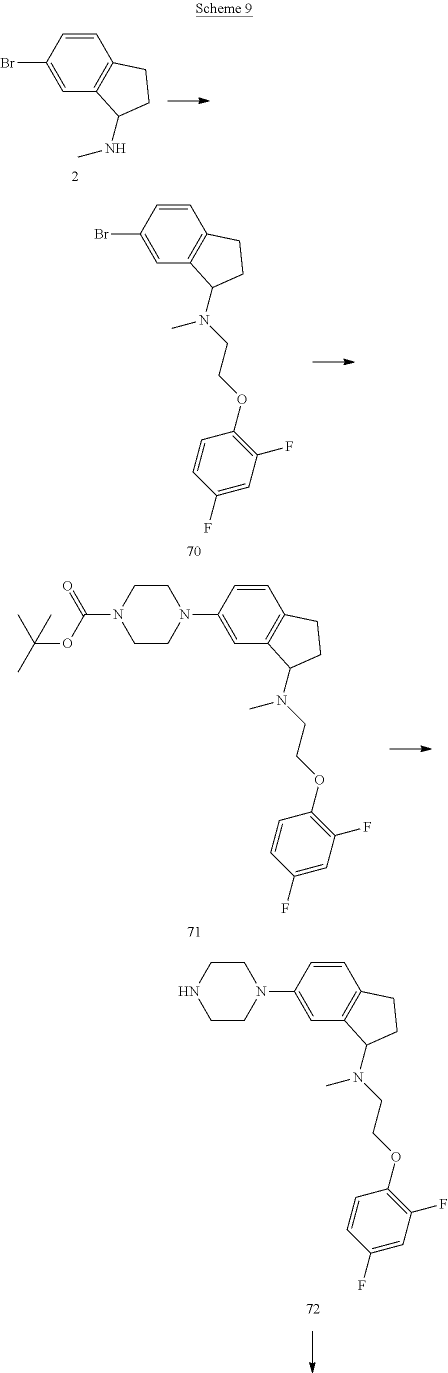 [SMILES: CNC1CCc2ccc(Br)cc21 ; CN(CCOc1ccc(F)cc1F)C1CCc2ccc(Br)cc21 ; CN(CCOc1ccc(F)cc1F)C1CCc2ccc(N3CCN(C(=O)OC(C)(C)C)CC3)cc21 ; CN(CCOc1ccc(F)cc1F)C1CCc2ccc(N3CCNCC3)cc21]

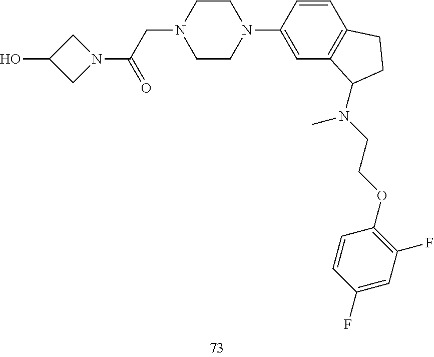 [SMILES: CN(CCOc1ccc(F)cc1F)C1CCc2ccc(N3CCN(CC(=O)N4CC(O)C4)CC3)cc21]

For the preparation of Compounds 73 and 74, please refer to Scheme 9 and the following details:

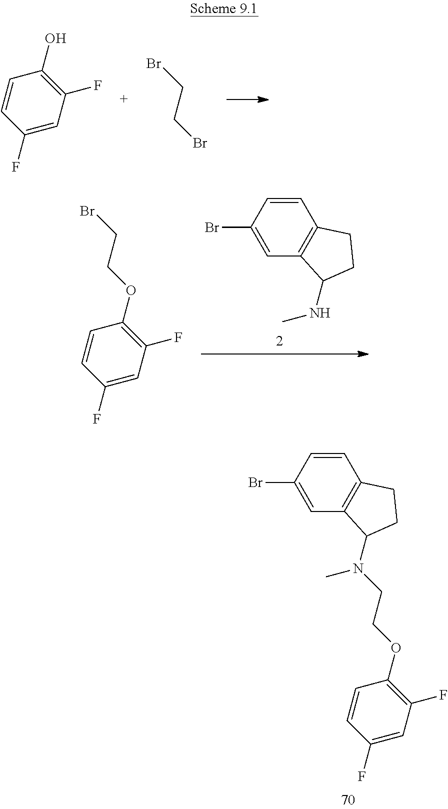

A suspension of 2,4-difluorophenol (8 g, 0.0615 mol), 1,2-dibromoethane (37.5 mL, 81.75 g, 0.435 mol) and K₂CO₃ (27.49 g, 0.198 mol) in acetonitrile (80 mL) was stirred at 60° C. overnight under a nitrogen atmosphere to form a reaction mixture. The reaction mixture was filtered and the filtrate was concentrated under reduced pressure. The concentrated residue was purified by column chromatography over silica gel, using 5% ethyl acetate in petroleum ether as eluent, to give an intermediate compound (9.93 g), 1-(2-Bromoethoxy)2,4-difluorobenzene, as a colorless liquid, and the yield thereof was 9.93 g. A mixture of 6-bromo-N-methyl-2,3-dihydro-1H-inden-1-amine (0.5 g, 2.2 mmol), the above intermediate compound (1-(2-Bromoethoxy)2,4-difluorobenzene) (0.79 g, 3.3 mmol), KI (0.35 g, 2.2 mmol) and potassium carbonate (0.73 g, 5.28 mmol) in dry N,N′-dimethylformamide (15 mL) was stirred overnight at 100° C. The mixture was vacuum concentrated and the residue was dissolved in water (25 mL), extracted with ethyl acetate (3×20 mL) and washed with water, and the resulting organic layers were combined. The organic layer was dried and evaporated to give a crude product. The crude product was purified via flash column chromatography on a silica gel column using 5:1 to 2:1 n-Hex-EtOAc as the eluent to give Compound 70, 6-bromo-N-(2-(2,4-difluorophenoxy)ethyl)-N-methyl-2,3-dihydro-1H-inden-1-amine (0.516 g, 61%).

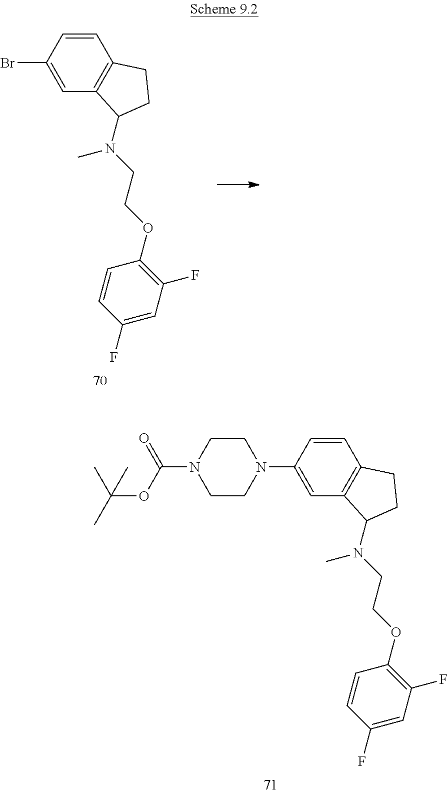 [SMILES: CN(CCOc1ccc(F)cc1F)C1CCc2ccc(Br)cc21 ; CN(CCOc1ccc(F)cc1F)C1CCc2ccc(N3CCN(C(=O)OC(C)(C)C)CC3)cc21]

CsCO₃ (0.623 g, 1.91 mmol), Boc-piperazine (0.34 g, 1.83 mmol), 2-(di-t-butylphosphino)biphenyl (0.046 g, 0.15 mmol) and Pd(OAc)₂ (0.041 g, 0.18 mmol) were added to a solution of Compound 70 (6-bromo-N-(2-(2,4-difluorophenoxy)ethyl)-N-methyl-2,3-dihydro-1H-inden-1-amine) (0.585 g, 1.53 mmol) in toluene (3 mL) to form a mixture. The mixture was degassed with argon for 15 minutes, and then heated at 80° C. overnight. After the reaction was completed, the solvent in the mixture was removed under reduced pressure, and then the residue was filtered through celite and washed with EtOAc to obtain a crude product. After concentration in vacuo, the crude product was purified via flash column chromatography on a silica gel column using 1:1 Hexane-EtOAc as the eluent to give Compound 71, tert-butyl 4-(3-((2-(2,4-difluorophenoxy)ethyl)(methyl)amino)-2,3-dihydro-1H-inden-5-yl)piperazine-1-carboxylate (0.26 g, 35%).

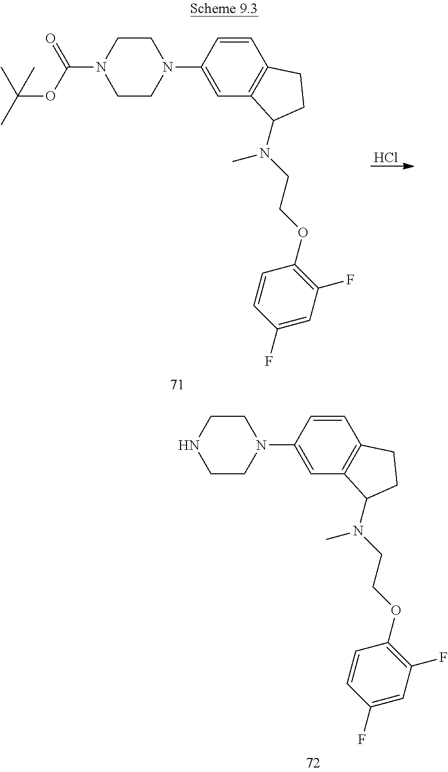 [SMILES: CN(CCOc1ccc(F)cc1F)C1CCc2ccc(N3CCN(C(=O)OC(C)(C)C)CC3)cc21 ; Cl ; CN(CCOc1ccc(F)cc1F)C1CCc2ccc(N3CCNCC3)cc21]

4 N HCl (in 1,4-dioxane, 1.5 mL) was added to a solution of Compound 71 (tert-butyl 4-(3-((2-(2,4-difluorophenoxy)ethyl)(methyl)amino)-2,3-dihydro-1H-inden-5-yl)piperazine-1-carboxylate) (258 mg, 0.529 mmol) in dry CH₂Cl₂ (4.0 mL) at 0° C. to form a mixture. The mixture was stirred at room temperature overnight. TLC was used to confirm the completion of reaction. After that, saturate NaHCO₃ was added to the mixture, and then the mixture was extracted with CH₂Cl₂. The resulting organic phase was dried with Na₂SO₄ and concentrated under reduced pressure to give a crude product which was brown (226 mg, 99%). The crude product was used as Compound 72, N-(2-(2,4-difluorophenoxy)ethyl)-N-methyl-6-(piperazin-1-yl)-2,3-dihydro-1H-inden-1-amine in the next step without further purification.

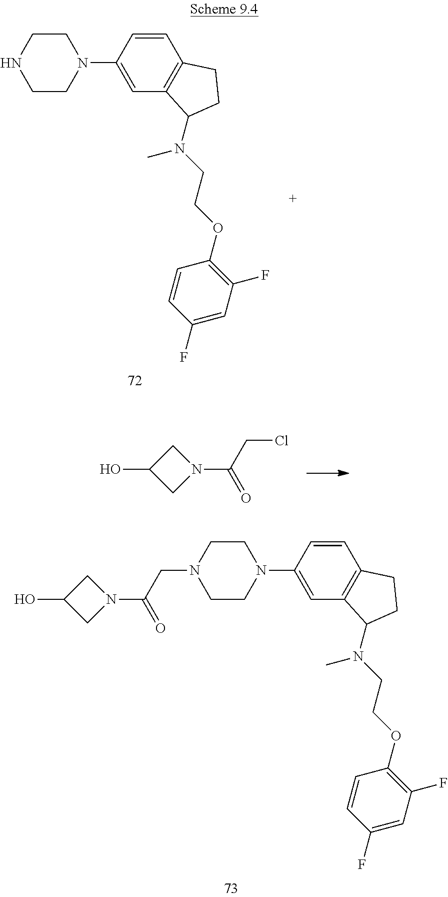

K₂CO₃ (0.07 g, 0.15 mmol), 2-chloro-1-(3-hydroxyazetidin-1-yl)ethan-1-one (0.05, 0.32 mmol) were added to a solution of Compound 72 (N-(2-(2,4-difluorophenoxy)ethyl)-N-methyl-6-(piperazin-1-yl)-2,3-dihydro-1H-inden-1-amine) (0.10 g) in MeCN (2 mL), and then the reaction mixture was heated at 80° C. for 5 hours. After the reaction was completed, the solvent in the reaction mixture was removed under reduced pressure. The residue was diluted with water and extracted with EtOAc, and the resulting organic were combined. The combined organic layers were dried over MgSO₄ and concentrated in vacuo to obtain a crude product. The crude product was purified via flash column chromatography on a silica gel column using 20:1 DCM-MeOH as the eluent to give Compound 73, and the yield thereof was 32 mg (25%).

Spectral Data of Compounds 73 and 74

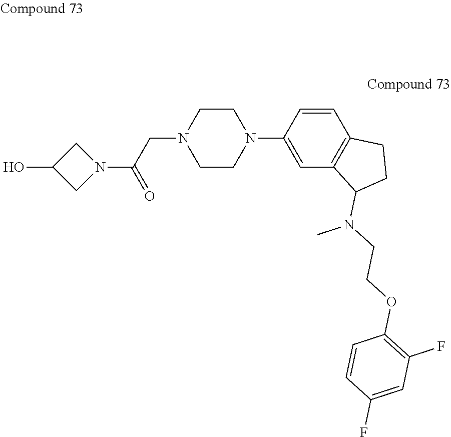 [SMILES: CN(CCOc1ccc(F)cc1F)C1CCc2ccc(N3CCN(CC(=O)N4CC(O)C4)CC3)cc21]

Compound 73, ¹H-NMR (400 MHz, CDCl₃): 7.10 (d, 1H), 6.99 (s, 1H), 6.91-6.81 (m, 3H), 6.79-6.73 (m, 1H), 4.70-4.66 (m, 1H), 4.45 (q, 2H), 4.28 (dd, 1H), 4.12-4.05 (m, 3H), 3.90 (dd, 1H), 3.20-3.12 (m, 4H), 3.08 (d, 2H), 2.92-2.81 (m, 2H), 2.78-2.72 (m, 2H), 2.70-2.64 (m, 4H), 2.40 (s, 3H), 2.18-2.11 (m, 1H), 2.09-1.81 (m, 2H). ESI-MS m/z calcd for C₂₇H₃₄F₂N₄O₃ 500.26, found 501.3 [M+H]⁺

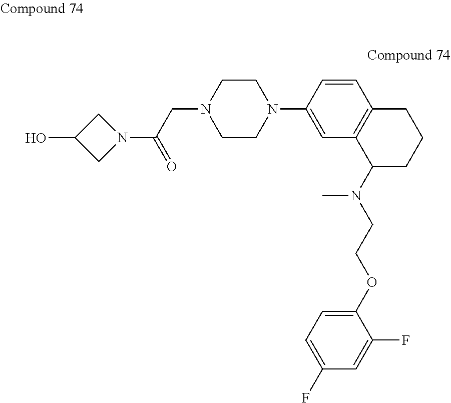 [SMILES: CN(CCOc1ccc(F)cc1F)C1CCCc2ccc(N3CCN(CC(=O)N4CC(O)C4)CC3)cc21]

Compound 74, ¹H-NMR (300 MHz, CDCl₃): 7.34 (s, 1H), 6.95 (d, 1H), 6.88-6.82 (m, 2H), 6.81-6.73 (m, 2H), 4.68 (br, 1H), 4.47 (t, 1H), 4.30 (t, 1H), 4.13-4.05 (m, 3H), 3.93-3.88 (m, 2H), 3.18-3.14 (m, 4H), 3.08 (s, 2H), 2.84 (m, 2H), 2.65-2.62 (m, 6H), 2.39 (s, 3H), 2.25 (br, 1H), 2.05-1.96 (m, 2H), 1.58 (m, 2H). ESI-MS m/z calcd for C₂₈H₃₆F₂N₄O₃ 514.28, found 515.4 [M+H]⁺

### Example 11. Compound 82

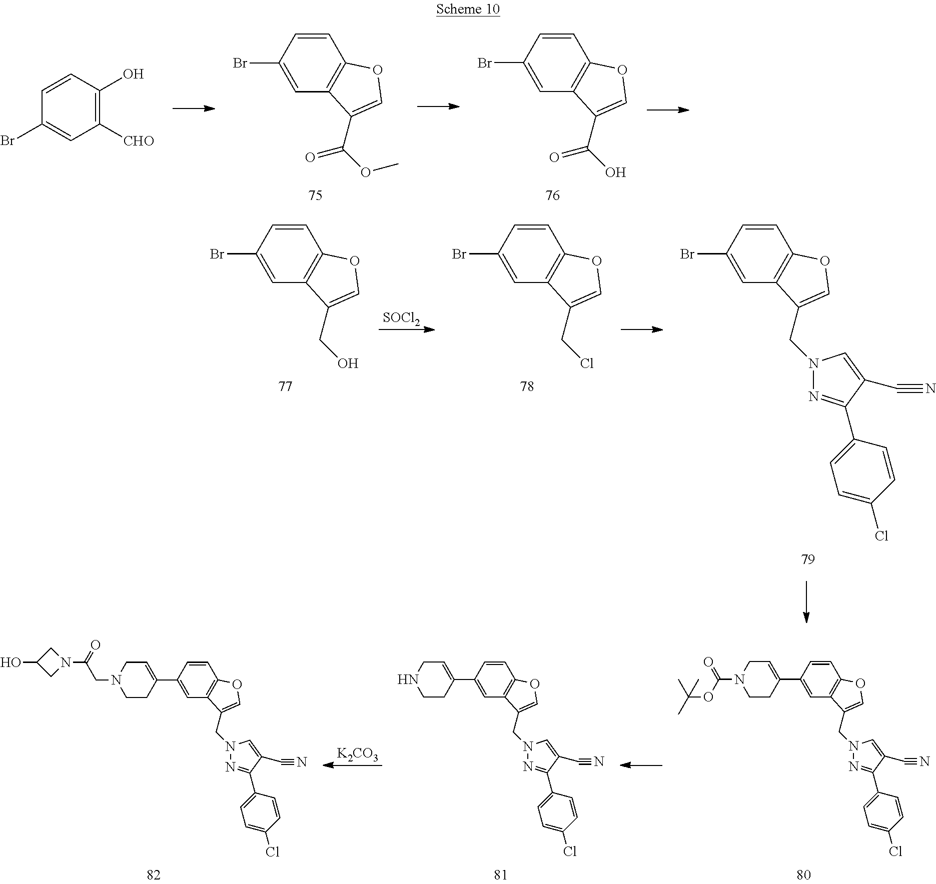

For the preparation of Compound 82, please refer to Scheme 10 and the following details:

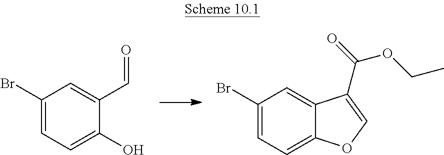

Ethyl diazoacetate (1.38 g, 12.09 mmol) dropwise was added to a mixture of 5-bromo-2-hydroxybenzaldehyde (1.21 g, 6.05 mmol) and 50% w/w HBF₄.Et₂O (0.20 g, 0.60 mmol) in DCM (30 mmol) at 0° C. to form a reaction mixture. After addition, the reaction mixture was slowly warmed to room temperature and stirred for 1 hour. After the hemiacetal intermediate was formed, concentrated H₂SO₄ (3 mL) was added into the reaction mixture and stirred for further 1 hour. After the reaction was completed, the solvent in the reaction mixture was removed under reduced pressure. The residual was neutralized with saturated NaHCO₃ and extracted with EtOAc; and the resulting organic layers were combined. The combined organic layers were dried over MgSO₄ and concentrated in vacuo to obtain a crude product. The crude product was used as Compound 75, ethyl 5-bromobenzofuran-3-carboxylate, in the next step without further purification.

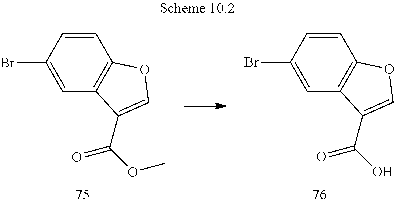

1N LiOH (6 mmol) was added to a solution of ethyl 5-bromobenzofuran-3-carboxylate (1.00 g, 3.72 mmol) in THF (20 mmol) and stirred overnight at room temperature to form a mixture. After the reaction was completed, the solvent in the mixture was removed under reduced pressure. The residue was acidified with 1N HCl, and the precipitated solid therefrom was collected by filtration and washed with cold water and hexane to obtain a crude product. The crude product acid was used as Compound 76, 5-bromobenzofuran-3-carboxylic, in the next step without further purification.

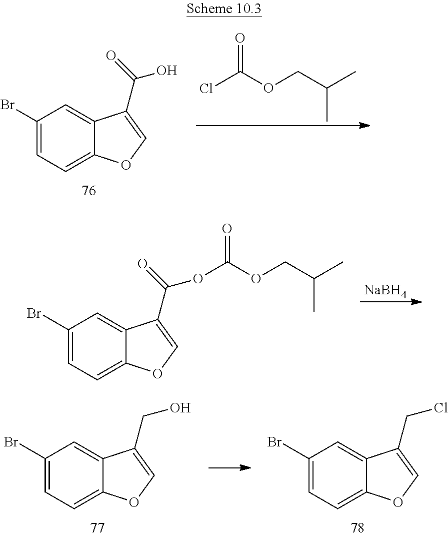

TEA (0.51 g, 5.02 mmol) and isobutyl chloroformate (0.68 g, 4.98 mmol) were added to a solution of 5-bromobenzofuran-3-carboxylic acid (1.00 g, 4.15 mmol) in THF (20 mL) at 0° C. and stirred for 2 hours at the same temperature to obtain a mixture. After the reaction was completed, the solvent in the mixture was removed under reduced pressure. The residue was dissolved in EtOAc and washed with 1N HCl, saturated NaHCO₃ and brine, and the resulting organic layers were combined. The combined organic layers were dried over MgSO₄ and concentrated in vacuo to obtain a crude mixed anhydride. The crude mixed anhydride was used in the next step without further purification.

NaBH₄ (0.16 g, 4.15 mmol) was added to a solution of mixed anhydride in MeOH (20 mL) at 0° C. After addition, the reaction mixture was slowly warmed to room temperature and stirred overnight. After the reaction was completed, the solvent in the mixture was removed under reduced pressure. The residue was diluted with saturated NH₄Cl and extracted with EtOAc, and the resulting organic layers were combined. The combined organic layers were dried over MgSO₄ and concentrated in vacuo to obtain crude (5-bromobenzofuran-3-yl)methanol. The crude (5-bromobenzofuran-3-yl)methanol was used in the next step without further purification. SOCl₂ (5 mL) was added to a solution of (5-bromobenzofuran-3-yl)methanol in Et₂O (20 mL) and stirred for 3 hours at room temperature to form a mixture. After the reaction was completed, the solvent in the mixture was removed under reduced pressure to obtain a crude product. The crude product was purified via flash column chromatography on a silica gel column using 10:1 Hexane-EtOAc as the eluent to give Compound 78, 5-bromo-3-(chloromethyl) benzofuran (0.56 g, 2.26 mmol).

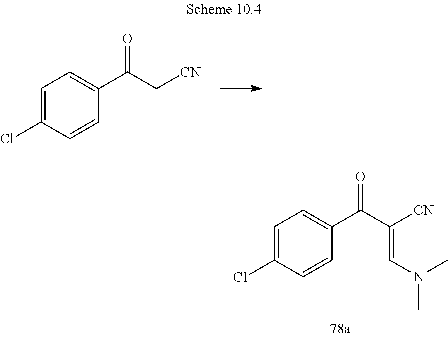 [SMILES: N#CCC(=O)c1ccc(Cl)cc1 ; CN(C)/C=C(\C#N)C(=O)c1ccc(Cl)cc1]

The 3-(4-chlorophenyl)-3-oxopropanenitrile (1 g, 5.58 mmol) was dissolved in N,N-Dimethylformamide dimethyl acetal (2 mL) and stirred at room temperature for 1 hour. After reaction was completed, the solvent was removed under reduced pressure. The residual was dissolved in EtOAc and washed with water and brine. The combined organic layers were dried over MgSO₄ and concentrated in vacuo. The crude product 78a, (E)-2-(4-chlorobenzoyl)-3-(dimethylamino)acrylonitrile (1.15 g, ˜88%), was used in the next step without further purification.

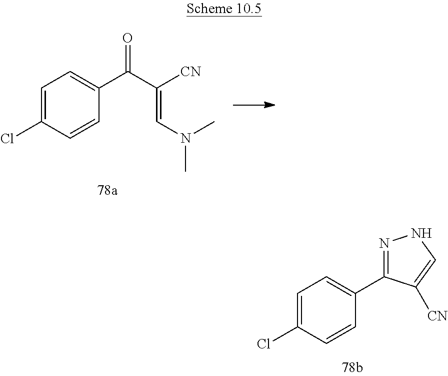 [SMILES: CN(C)/C=C(\C#N)C(=O)c1ccc(Cl)cc1 ; N#Cc1c[nH]nc1-c1ccc(Cl)cc1]

To a solution of Compound 78a ((E)-2-(4-chlorobenzoyl)-3-(dimethylamino)acrylonitrile) (1.15 g, 4.9 mmol) in EtOH (10 mL) was added conc. HCl (0.1 mL) and hydrazine hydrate (0.3 g). After addition, the reaction mixture was refluxed for 3 hours. After reaction was completed, the solvent was removed under reduced pressure. The residual was diluted with cold water and sit to get a precipitated solid. The precipitated solid was collected by filtration to obtain a crude product of Compound 78b (0.81 g, yield=81%). Compound 78b was used in the next step without further purification.

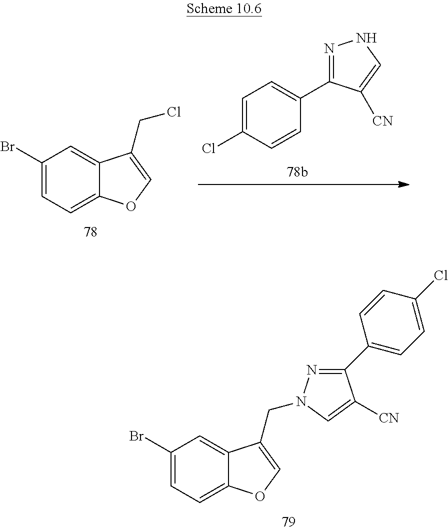 [SMILES: ClCc1coc2ccc(Br)cc12 ; N#Cc1c[nH]nc1-c1ccc(Cl)cc1 ; N#Cc1cn(Cc2coc3ccc(Br)cc23)nc1-c1ccc(Cl)cc1]

NaH (0.18 g) was added to a solution of Compound 78b (0.16 g, 0.79 mmol) in DMF (10 mL) at 0° C. and stirred for 30 minutes at the same temperature to form a reaction mixture, and then the Compound 78 (5-bromo-3-(chloromethyl)benzofuran) (0.16 g, 0.65 mmol) was added into the reaction mixture. After addition, the reaction mixture was slowly warmed to room temperature and stirred overnight. After the reaction was completed, the solvent in the reaction mixture was evaporated off by air-drying, and then the residue was diluted with saturated NH₄Cl and extracted with EtOAc, and the resulting organic layers were combined. The combined organic layers were dried over MgSO₄ and concentrated in vacuo to produce a crude product. The crude product was purified via flash column chromatography on a silica gel column using 5:1 Hexane-EtOAc as the eluent to give Compound 79, 1-((5-bromobenzofuran-3-yl)methyl)-3-(4-chlorophenyl)-1H-pyrazole-4-carbonitrile (0.18 g, 0.42 mmol).

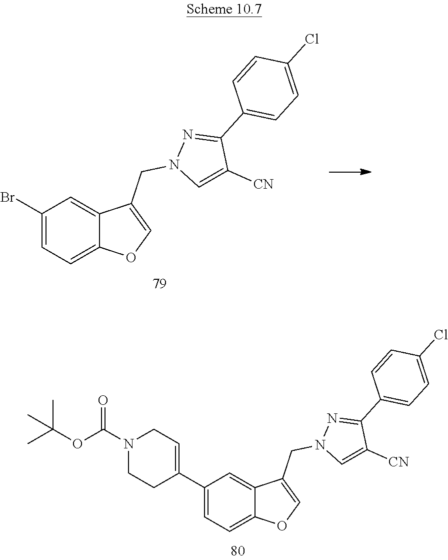 [SMILES: N#Cc1cn(Cc2coc3ccc(Br)cc23)nc1-c1ccc(Cl)cc1 ; CC(C)(C)OC(=O)N1CC=C(c2ccc3occ(Cn4cc(C#N)c(-c5ccc(Cl)cc5)n4)c3c2)CC1]

Na₂CO₃ (0.29 g, 2.74 mmol), boronic ester (0.56 g, 1.81 mmol) and Pd(dppf)Cl₂ (0.07 g, 0.09 mmol) were added to a solution of 1-((5-bromobenzofuran-3-yl)methyl)-3-(4-chlorophenyl)-1H-pyrazole-4-carbonitrile (0.37 g, 0.90 mmol) in DMF (10 mL). The mixture was degassed with Ar for 15 minutes, and then heated at 100° C. overnight. After the reaction was completed, the solvent in the mixture was evaporated off by air-drying, and then the residue was filtered through celite and washed with EtOAc to obtain a crude product. After concentration in vacuo, the crude product was purified via flash column chromatography on a silica gel column using 5:1 Hexane-EtOAc as the eluent to give Compound 80, tert-butyl 4-(3-((3-(4-chlorophenyl)-4-cyano-1H-pyrazol-1-yl)methyl)benzofuran-5-yl)-3,6-dihydropyridine-1(2H)-carboxylate (0.26 g, 0.50 mmol).

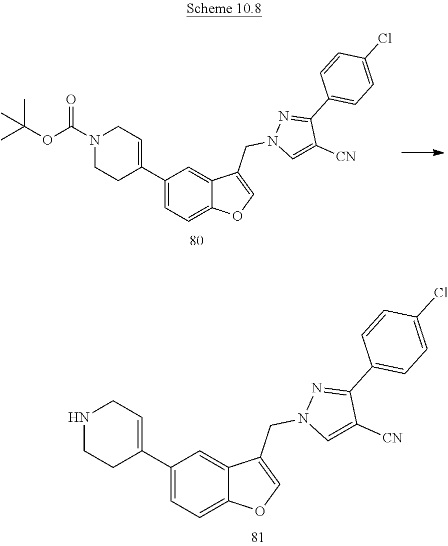 [SMILES: CC(C)(C)OC(=O)N1CC=C(c2ccc3occ(Cn4cc(C#N)c(-c5ccc(Cl)cc5)n4)c3c2)CC1 ; N#Cc1cn(Cc2coc3ccc(C4=CCNCC4)cc23)nc1-c1ccc(Cl)cc1]

Tert-butyl 4-(3-((3-(4-chlorophenyl)-4-cyano-1H-pyrazol-1-yl)methyl)benzofuran-5-yl)-3,6-dihydropyridine-1(2H)-carboxylate (0.26, 0.50 mmol) was added to a solution of 4N HCl in dioxane (10 mL), and then stirred for 3 hours to form a mixture. After the reaction was completed, the solvent in the mixture was removed under reduced pressure to obtain a crude product. The crude product was used as Compound 81, 3-(4-chlorophenyl)-1-((5-(1,2,3,6-tetrahydropyridin-4-yl)benzofuran-3-yl)methyl)-1H-pyrazole-4-carbonitrile, in the next step without further purification.

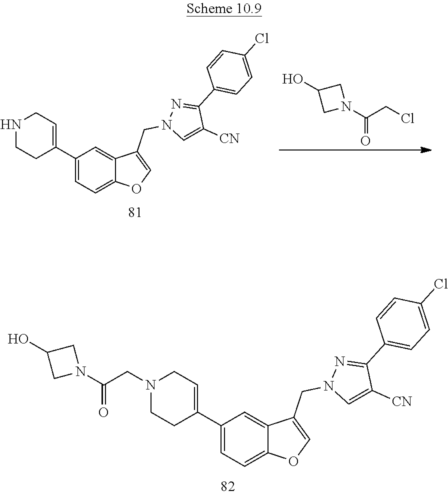 [SMILES: N#Cc1cn(Cc2coc3ccc(C4=CCNCC4)cc23)nc1-c1ccc(Cl)cc1 ; O=C(CCl)N1CC(O)C1 ; N#Cc1cn(Cc2coc3ccc(C4=CCN(CC(=O)N5CC(O)C5)CC4)cc23)nc1-c1ccc(Cl)cc1]

K₂CO₃ (0.20 g, 1.45 mmol), alkyl chloride (0.07 g, 0.47 mmol) and a catalytic amount of KI were added to a solution of 3-(4-chlorophenyl)-1-((5-(1,2,3,6-tetrahydropyridin-4-yl)benzofuran-3-yl)methyl)-1H-pyrazole-4-carbonitrile in DMF (3 mL), and then the reaction mixture was heated at 80° C. overnight. After the reaction was completed, the solvent in the reaction mixture was evaporated off by air-drying. The residue was diluted with water and extracted with EtOAc, and the resulting organic layers were combined. The combined organic layers were dried over MgSO₄ and concentrated in vacuo to obtain a crude product. The crude product was purified via flash column chromatography on a silica gel column using 10:1 EtOAc-MeOH as the eluent to give, Compound 82, 3-(4-chlorophenyl)-1-((5-(1-(2-(3-hydroxyazetidin-1-yl)-2-oxoethyl)-1,2,3,6-tetrahydropyridin-4-yl)benzofuran-3-yl)methyl)-1H-pyrazole-4-carbonitrile, and the yield thereof was 0.09 g (0.17 mmol).

Spectral Data of Compound 82

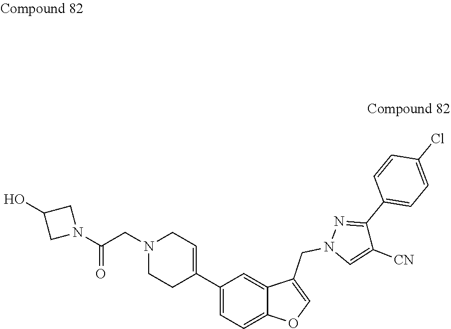 [SMILES: N#Cc1cn(Cc2coc3ccc(C4=CCN(CC(=O)N5CC(O)C5)CC4)cc23)nc1-c1ccc(Cl)cc1]

Compound 82, ¹H-NMR (300 MHz, CDCl₃): 7.96-7.93 (m, 2H), 7.84 (s, 1H), 7.75 (s, 1H), 7.48-7.41 (m, 5H), 5.98 (s, 1H), 5.48 (s, 2H), 4.69-4.65 (m, 1H), 4.49-4.44 (m, 1H), 4.32-4.27 (m, 1H), 4.13-4.09 (m, 1H), 3.93-3.88 (m, 1H), 3.22-3.16 (m, 4H), 2.78-2.75 (t, 2H), 2.56 (s, 2H). 1.92-1.6 (br, 1H). ESI-MS m/z calcd for C₂₉H₂₆ClN₅O₃ 527.17, found 528.3 [M+H]⁺.

### Example 12. Compound 85

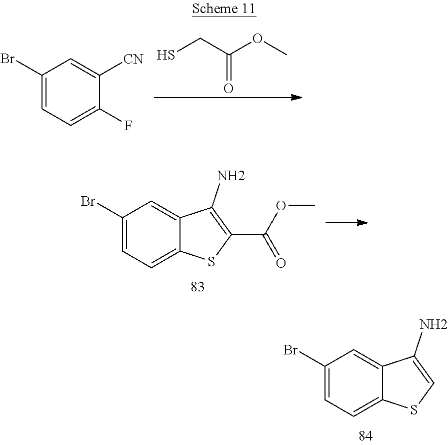

For the preparation of Compound 85, please refer to Scheme 11 and the following details:

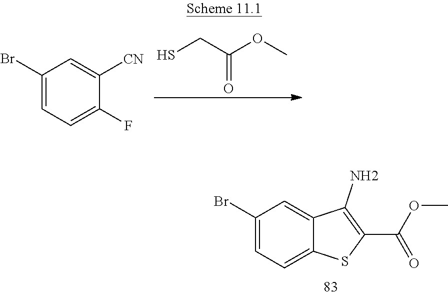

To a cold solution of 5-bromo-2-fluorobenzonitrile (7.43 g, 37.15 mmol) in DMF at 0° C. was added dropwise methyl 2-mercaptoacetate (6.65 mL, 74.3 mmol). The reaction mixture was stirred at 0° C. for 30 minutes, and then potassium tert-butoxide (8.4 g, 74.5 mmol) was added over 15 minutes with vigorous stirring. After stirring at 0° C. for 0.5 hour, allowed to warm to room temperature and stirred for 3 hours. The reaction mixture was quenched with ice-water. The resulting precipitate was collected by filtration and dried to give 9.8 g of white solid of Compound 83 (methyl 3-amino-5-bromobenzo[b]thiophene-2-carboxylate) in 92% yield.

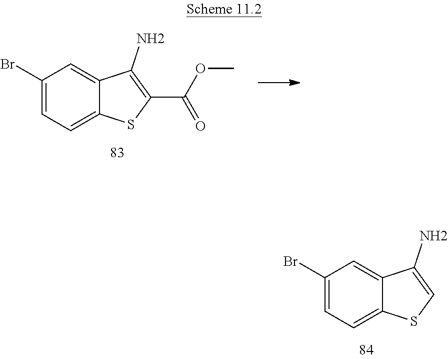

To a solution of Compound 83 (methyl 3-amino-5-bromobenzo[b]thiophene-2-carboxylate) (0.76 g, 2.66 mmol, 1 eq) in 1-methyl-2-pyrrolidinone (4.5 mL) was added piperazine (1.56 mL, 14.1 mmol, 5.3 eq). The reaction was stirred at 130° C. overnight. Ice was added, and the mixture was extracted with ethyl acetate. The organic extracts were washed twice with water, dried, and concentrated in vacuo. The crude material was purified by flash chromatography on silica gel eluting with 30% ethyl acetate in hexanes to provide Compound 84 (5-bromobenzo[b]thiophen-3-amine) (460 mg, 76%).

Compound 85 was produced by starting with Compound 84 and followed by Schemes 2.1, 7.2, and 8.1 to 8.3, accordingly.

Spectral Data of Compound 85

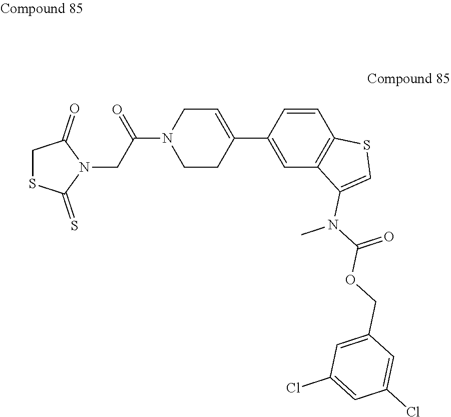 [SMILES: CN(C(=O)OCc1cc(Cl)cc(Cl)c1)c1csc2ccc(C3=CCN(C(=O)CN4C(=O)CSC4=S)CC3)cc12]

Compound 85, 1H-NMR (400 MHz, CDCl₃): 7.81 (s, 1H), 7.45-7.26 (m, 5H), 6.94 (br, 1H), 6.06-5.99 (m, 1H), 5.03 (br, 2H), 4.91 (s, 1H), 4.86 (s, 1H), 4.24 (s, 2H), 4.10 (s, 2H), 3.84-3.75 (m, 2H), 3.39 (s, 3H), 2.67-2.56 (m, 2H) ESI-MS m/z calcd for C₂₇H₂₃Cl₂N₃O₄S₃ 619.02, found 642.3 [M+Na]⁺.

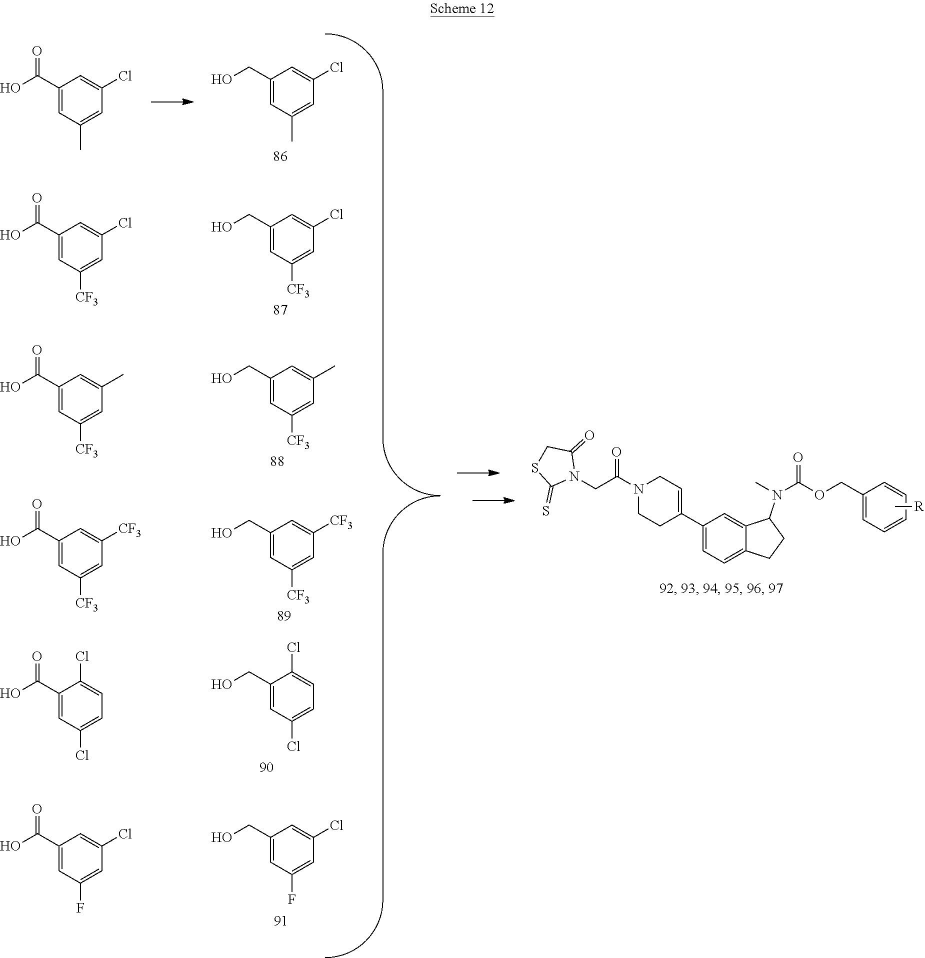

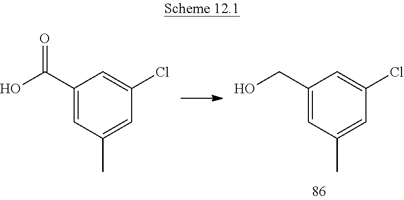

To a solution of LAH (0.17 g, 4.40 mmol) in THF (30 mL) was added 3-Chloro-5-methylbenzoic acid (0.50 g, 2.93 mmol) slowly at 0° C. After addition, the reaction mixture was slowly warmed to RT and stirred for 3 hours. After reaction was completed, the solvent was removed under reduced pressure. The residue was quenched with 1N HCl and extracted with EtOAc. The combined organic layers were dried over MgSO₄ and concentrated in vacuo to afford Compound 86 (3-Chloro-5-methylbenzyl alcohol) (0.45 g, 2.87 mmol, 98% yield). The crude product was used in the next step without further purification.

Compound 87, 88, 89, 90 and 91 are synthesized by using the same method as in Scheme 12.1.

Compound 92, 93, 94, 95, 96 and 97 are synthesized by using the same methods as in Scheme 1.3, 1.4, 1.5 and 1.8 with Compound 86, 87, 88, 89, 90 and 91, respectively.

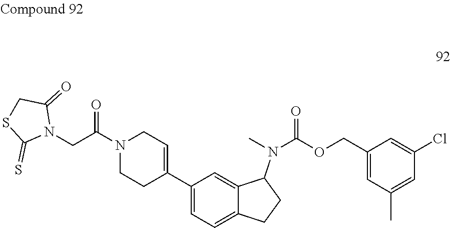 [SMILES: Cc1cc(Cl)cc(COC(=O)N(C)C2CCc3ccc(C4=CCN(C(=O)CN5C(=O)CSC5=S)CC4)cc32)c1]

Compound 92, ¹H-NMR (300 MHz, CDCl₃): 7.27-7.06 (m, 6H), 6.02-5.99 (d, 2H), 5.94-5.76 (m, 1H), 5.18-5.08 (m, 2H), 4.91-4.85 (d, 2H), 4.23 (s, 2H), 4.14 (s, 2H), 3.81-3.72 (m, 2H), 3.03-2.86 (m, 2H), 2.69-2.66 (m, 3H), 2.53-2.35 (m, 3H), 2.35 (s, 3H) ESI-MS m/z calcd for C₂₉H₃₀ClN₃O₄S₂ 583.14, found 606.5 [M+Na]⁺

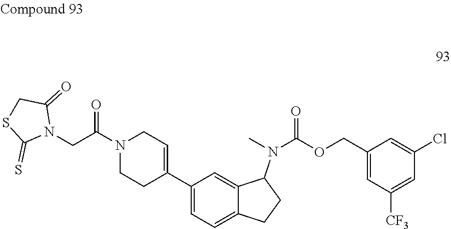 [SMILES: CN(C(=O)OCc1cc(Cl)cc(C(F)(F)F)c1)C1CCc2ccc(C3=CCN(C(=O)CN4C(=O)CSC4=S)CC3)cc21]

Compound 93, ¹H-NMR (400 MHz, CDCl₃): 7.58-7.51 (m, 2H), 7.29-7.10 (m, 4H), 6.03-5.99 (d, 1H), 5.91-5.73 (m, 1H), 5.28-5.21 (m, 2H), 4.91-4.84 (d, 2H), 4.23 (s, 2H), 4.10 (s, 2H), 3.83-3.72 (m, 2H), 3.03-2.84 (m, 2H), 2.68 (s, 3H), 2.54-2.45 (m, 3H), 2.01-1.93 (m, 1H) ESI-MS m/z calcd for C₂₉H₂₇ClF₃N₃O₄S₂ 637.11, found 638.1 [M+H]⁺

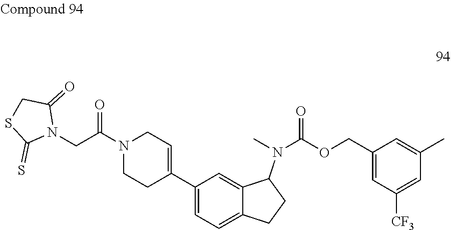 [SMILES: Cc1cc(COC(=O)N(C)C2CCc3ccc(C4=CCN(C(=O)CN5C(=O)CSC5=S)CC4)cc32)cc(C(F)(F)F)c1]

Compound 94, ¹H-NMR (400 MHz, CDCl₃): 7.52-7.10 (m, 6H), 6.03-5.98 (d, 1H), 5.93-5.75 (m, 1H), 5.27-5.19 (m, 2H), 4.90-4.84 (d, 2H), 4.22 (s, 2H), 4.11 (s, 2H), 3.81-3.71 (m, 2H), 3.02-2.85 (m, 2H), 2.67 (s, 3H), 2.52-2.42 (m, 3H), 2.44 (s, 3H), 2.02-1.95 (m, 1H) ESI-MS m/z calcd for C₃₀H₃₀F₃N₃O₄S₂ 617.16, found 640.1 [M+Na]⁺

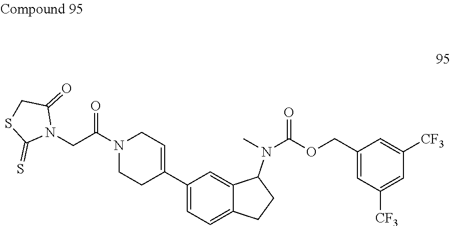 [SMILES: CN(C(=O)OCc1cc(C(F)(F)F)cc(C(F)(F)F)c1)C1CCc2ccc(C3=CCN(C(=O)CN4C(=O)CSC4=S)CC3)cc21]

Compound 95, ¹H-NMR (400 MHz, CDCl₃): 7.85-7.82 (m, 3H), 7.28-7.11 (m, 3H), 6.03-5.98 (d, 1H), 5.91-5.75 (m, 1H), 5.33 (s, 2H), 4.90-4.84 (d, 2H), 4.22 (s, 2H), 4.10 (s, 2H), 3.83-3.72 (m, 2H), 3.04-2.86 (m, 2H), 2.69 (s, 3H), 2.65-2.40 (m, 3H), 2.01-1.95 (m, 1H) ESI-MS m/z calcd for C₃₀H₂₇F₆N₃O₄S₂ 671.13, found 694.1 [M+Na]⁺

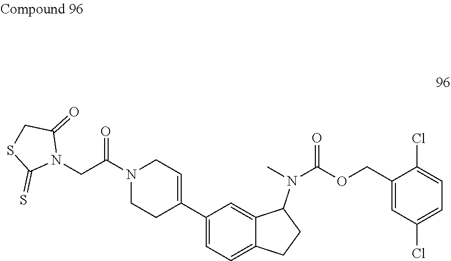 [SMILES: CN(C(=O)OCc1cc(Cl)ccc1Cl)C1CCc2ccc(C3=CCN(C(=O)CN4C(=O)CSC4=S)CC3)cc21]

Compound 96, ¹H-NMR (300 MHz, CDCl₃): 7.43-7.14 (m, 6H), 6.04-5.99 (d, 1H), 5.94-5.75 (m, 1H), 5.36-5.21 (m, 2H), 4.90-4.85 (d, 2H), 4.23 (s, 2H), 4.10 (s, 2H), 3.82-3.72 (m, 2H), 3.00-2.84 (m, 2H), 2.70 (s, 3H), 2.54-2.45 (m, 3H), 2.45-2.00 (m, 1H) ESI-MS m/z calcd for C₂₈H₂₇Cl₂N₃O₄S₂ 603.08, found 626.1 [M+Na]⁺

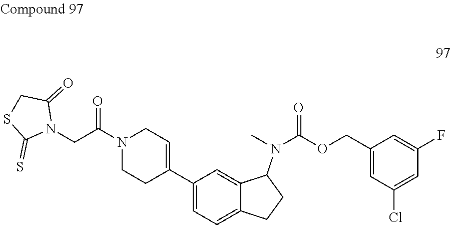 [SMILES: CN(C(=O)OCc1cc(F)cc(Cl)c1)C1CCc2ccc(C3=CCN(C(=O)CN4C(=O)CSC4=S)CC3)cc21]

Compound 97, 1H NMR (400 MHz, CDCl₃) δ 8.29 (d, 1H, J=9.2 Hz), 7.42-7.22 (m, 5H), 6.05 (d, 1H, J=15.6 Hz), 5.93-5.88 (m, 1H), 4.88 (d, 2H, J=24 Hz), 4.25 (s, 2H), 4.11 (s, 2H), 3.87-3.76 (m, 2H), 3.12-3.03 (m, 1H), 2.96-2.88 (m, 1H), 2.81 (d, 3H, 20.8 Hz), 2.74-2.64 (m, 1H), 2.57-2.50 (m, 2H), 2.17-2.02 (m, 2H). ESI-MS m/z calcd for C₂₈H₂₇ClFN₃O₄S₂ 587.111, found 588.1 [M+H]⁺.

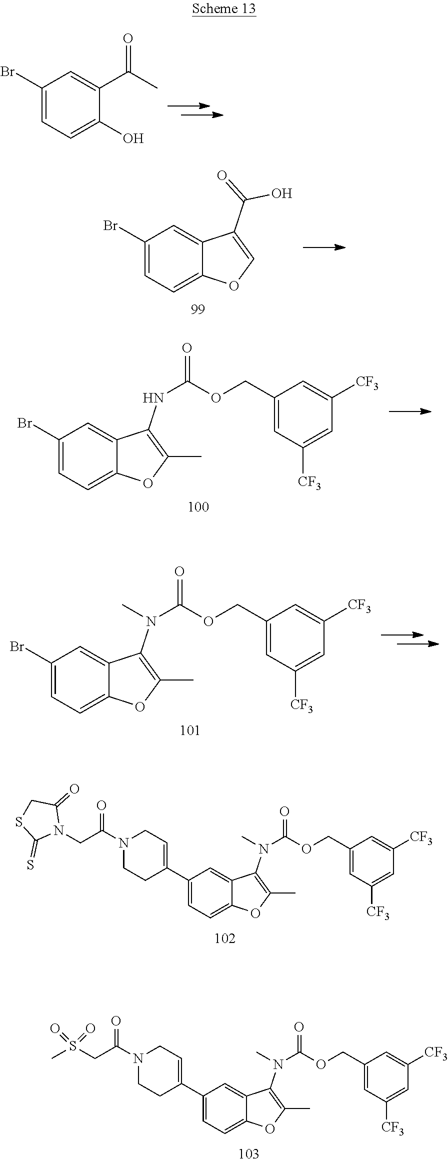 [SMILES: CC(=O)c1cc(Br)ccc1O ; O=C(O)c1coc2ccc(Br)cc12 ; Cc1oc2ccc(Br)cc2c1NC(=O)OCc1cc(C(F)(F)F)cc(C(F)(F)F)c1 ; Cc1oc2ccc(Br)cc2c1N(C)C(=O)OCc1cc(C(F)(F)F)cc(C(F)(F)F)c1 ; Cc1oc2ccc(C3=CCN(C(=O)CN4C(=O)CSC4=S)CC3)cc2c1N(C)C(=O)OCc1cc(C(F)(F)F)cc(C(F)(F)F)c1 ; Cc1oc2ccc(C3=CCN(C(=O)CS(C)(=O)=O)CC3)cc2c1N(C)C(=O)OCc1cc(C(F)(F)F)cc(C(F)(F)F)c1]

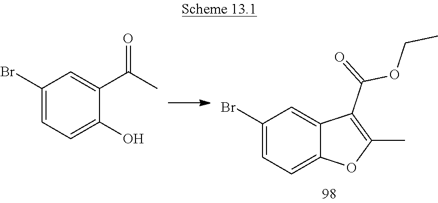 [SMILES: CC(=O)c1cc(Br)ccc1O ; CCOC(=O)c1c(C)oc2ccc(Br)cc12]

To a mixture of 5′-Bromo-2′-hydroxyacetophenone (2.1 g, 9.95 mmol) and HBF₄.Et₂O (0.32 g, 1 mmol) in DCM (15 mL) was slowly added a solution of ethyl diazoacetate (1.8 g, 15.77 mmol) in DCM (15 mL) at 0° C. After addition, the reaction mixture was slowly warmed to RT and stirred for 2 hours. Then the reaction mixture was added conc. H₂SO₄ (1.3 g) and stirred for further 20 min. After reaction was completed, the reaction mixture was neutralized with Na₂CO₃ and the solvent was removed under reduced pressure. The crude product was purified via flash column chromatography on a silica gel column using 10:1 Hexane-EtOAc as the eluent to give Compound 98 (ethyl 5-bromo-2-methylbenzofuran-3-carboxylate) (2.30 g, 8.12 mmol, 82% yield).

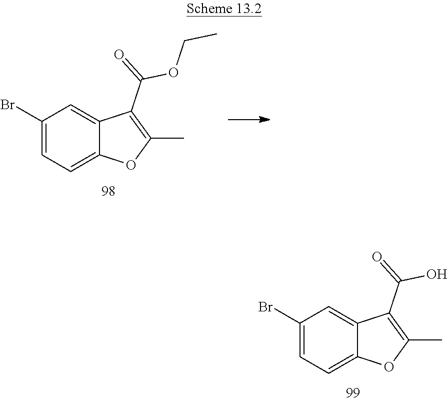 [SMILES: CCOC(=O)c1c(C)oc2ccc(Br)cc12 ; Cc1oc2ccc(Br)cc2c1C(=O)O]

To a solution of Compound 98 (0.74 g, 2.62 mmol) in THF (20 mL) and MeOH (20 mL) was added 1N LiOH (5 mL). After addition, the reaction mixture was heated to reflux for 2 hours. After reaction was completed, the solvent was removed under reduced pressure. The residual was acidified with 1N HCl. The precipitated solid was collected by filtration to afford Compound 99 (5-bromo-2-methylbenzofuran-3-carboxylic acid) (0.52 g, 2.06 mmol, yield 78%)

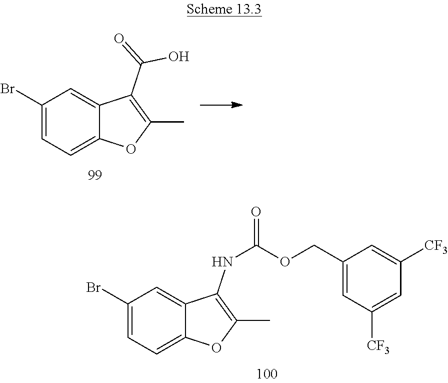 [SMILES: Cc1oc2ccc(Br)cc2c1C(=O)O ; Cc1oc2ccc(Br)cc2c1NC(=O)OCc1cc(C(F)(F)F)cc(C(F)(F)F)c1]

To a solution of Compound 99 (0.50 g, 1.96 mmol) and 3,5-Bis(trifluoromethyl)benzyl alcohol (0.42 g, 2.35 mmol) in toluene (30 mL) was added TEA (0.40 g, 3.92 mmol) and diphenylphosphorylazide (0.65 g, 2.35 mmol). After addition, the reaction mixture was heated to reflux for overnight. After reaction was completed, the solvent was removed under reduced pressure. The residual was diluted with EtOAc and washed with Sat. NH₄Cl, Sat. NaHCO₃ and brine. The combined organic layers were dried over MgSO₄ and concentrated in vacuo to afford Compound 100 (3,5-bis(trifluoromethyl)benzyl (5-bromo-2-methylbenzofuran-3-yl)carbamate) (0.94 g, 1.90 mmol, 97% yield). The product was used in next step without further purification.

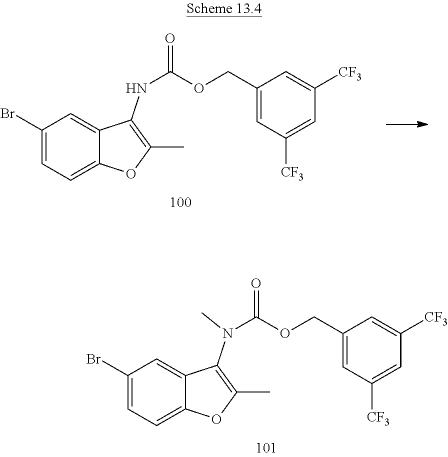 [SMILES: Cc1oc2ccc(Br)cc2c1NC(=O)OCc1cc(C(F)(F)F)cc(C(F)(F)F)c1 ; Cc1oc2ccc(Br)cc2c1N(C)C(=O)OCc1cc(C(F)(F)F)cc(C(F)(F)F)c1]

To a solution of Compound 100 (0.50 g, 1.01 mmol) in ACN (20 mL) was added NaH (0.06 g, 1.51 mmol) at 0° C. and stirred for 30 min at the same temperature, then MeI (0.50 mL) was added into the reaction mixture. After addition, the reaction mixture was slowly warmed to RT and stirred for 1 hr. After reaction was completed, the solvent was removed under reduced pressure. The residual was diluted with EtOAc and washed with Sat. NH₄Cl, Sat. NaHCO₃ and brine. The combined organic layers were dried over MgSO₄ and concentrated in vacuo. The crude product was purified via flash column chromatography on a silica gel column using 10:3 Hexane-EtOAc as the eluent to give Compound 101 (3,5-bis(trifluoromethyl)benzyl (5-bromo-2-methylbenzofuran-3-yl)(methyl)carbamate) (0.38 g, 0.74 mmol, 73% yield).

Compound 102 was synthesized by using the same methods as in Scheme 1.4, 1.5 and 1.8 with Compound 101. Compound 103 was synthesized by using the same methods for Compound 102.

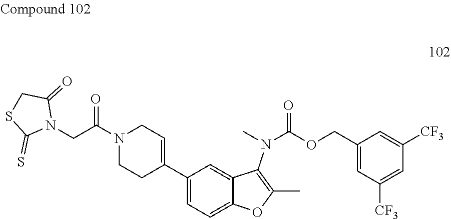 [SMILES: Cc1oc2ccc(C3=CCN(C(=O)CN4C(=O)CSC4=S)CC3)cc2c1N(C)C(=O)OCc1cc(C(F)(F)F)cc(C(F)(F)F)c1]

Compound 102, ¹H-NMR (400 MHz, CDCl₃): 7.91-7.21 (m, 6H), 6.01-5.96 (d, 1H), 5.37-5.19 (m, 2H), 4.91-4.85 (d, 2H), 4.23 (s, 2H), 4.10 (s, 2H), 3.85-3.73 (m, 2H), 3.32 (s, 3H), 2.69-2.58 (m, 2H), 2.35 (s, 3H) ESI-MS m/z calcd for C₃₀H₂₅F₆N₃O₅S₂ 685.11, found 708.1[M+Na]⁺

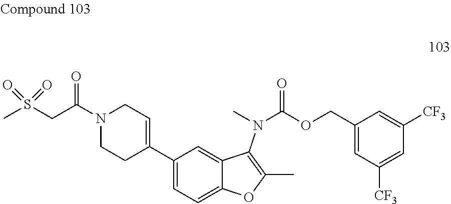 [SMILES: Cc1oc2ccc(C3=CCN(C(=O)CS(C)(=O)=O)CC3)cc2c1N(C)C(=O)OCc1cc(C(F)(F)F)cc(C(F)(F)F)c1]

Compound 103, ¹H-NMR (400 MHz, CDCl₃): 7.90-7.27 (m, 6H), 6.00-5.95 (d, 1H), 5.36-5.20 (m, 2H), 4.30 (s, 2H), 4.18-4.14 (d, 2H), 3.91-3.81 (m, 2H), 3.32 (s, 3H), 3.15 (s, 3H), 2.68-2.60 (m, 2H), 2.35 (s, 3H) ESI-MS m/z calcd for C₂₈H₂₆F₆N₂O₆S 632.14, found 655.1[M+Na]⁺

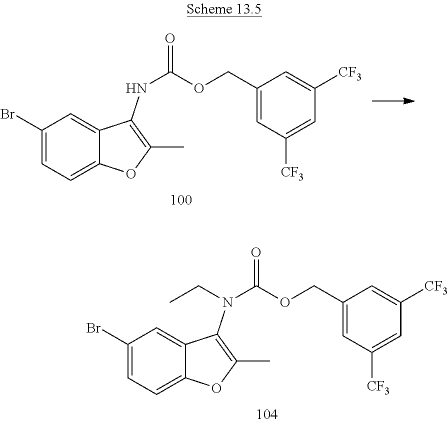 [SMILES: Cc1oc2ccc(Br)cc2c1NC(=O)OCc1cc(C(F)(F)F)cc(C(F)(F)F)c1 ; CCN(C(=O)OCc1cc(C(F)(F)F)cc(C(F)(F)F)c1)c1c(C)oc2ccc(Br)cc12]

Compound 104 was synthesized by using the same method in Scheme 13.4 with ethyl iodide.

Compound 105 was synthesized by using the same methods as in Scheme 1.4, 1.5 and 1.8 with Compound 104.

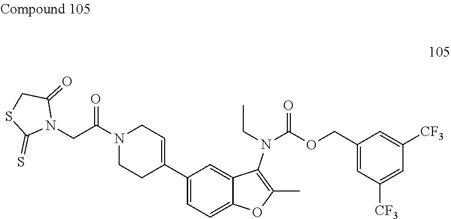 [SMILES: CCN(C(=O)OCc1cc(C(F)(F)F)cc(C(F)(F)F)c1)c1c(C)oc2ccc(C3=CCN(C(=O)CN4C(=O)CSC4=S)CC3)cc12]

Compound 105, ¹H-NMR (400 MHz, CDCl₃): 7.89-7.26 (m, 6H), 6.00-5.95 (d, 1H), 5.38-5.15 (m, 2H), 4.91-4.85 (d, 2H), 4.23 (s, 2H), 4.10 (s, 2H), 3.85-3.70 (m, 4H), 2.69-2.58 (m, 2H), 2.35 (s, 3H), 1.21-1.17 (t, 3H) ESI-MS m/z calcd for C₃₁H₂₇F₆N₃O₅S₂ 699.13, found 722.1 [M+Na]⁺

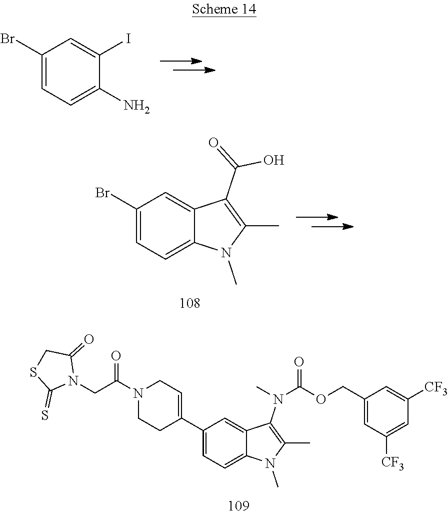 [SMILES: Nc1ccc(Br)cc1I ; Cc1c(C(=O)O)c2cc(Br)ccc2n1C ; Cc1c(N(C)C(=O)OCc2cc(C(F)(F)F)cc(C(F)(F)F)c2)c2cc(C3=CCN(C(=O)CN4C(=O)CSC4=S)CC3)ccc2n1C]

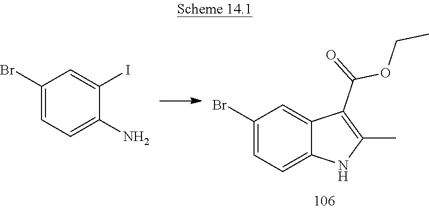 [SMILES: Nc1ccc(Br)cc1I ; CCOC(=O)c1c(C)[nH]c2ccc(Br)cc12]

To a mixture of 4-Bromo-2-iodoaniline (1.38 g, 4.6 mmol) ethyl acetoacetate (0.67 g, 5.1 mmol), CuI (0.1 g, 0.52 mmol), and BINOL (0.2 g, 0.70 mmol) was added Cs₂CO₃ (1.5 g, 4.60 mmol) in DMSO. After addition, the reaction mixture was heated at 50° C. for overnight. After reaction was completed, the reaction mixture was diluted with Sat. NH₄Cl and EtOAc. The organic layer was washed with brine and dried over MgSO₄ to afford Compound 106 (ethyl 5-bromo-2-methyl-1H-indole-3-carboxylate) (1.17 g, 4.15 mmol, yield 90%). The product was used in next step without further purification.

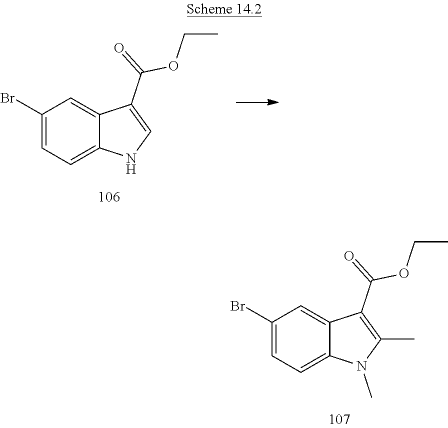 [SMILES: CCOC(=O)c1c[nH]c2ccc(Br)cc12 ; CCOC(=O)c1c(C)n(C)c2ccc(Br)cc12]

To a solution of Compound 106 (1.17 g, 4.15 mmol) in DMF was added NaH (0.25 g, 6.23 mmol) at 0° C. and stirred for 10 min, then to the reaction mixture was added MeI (0.88 g, 6.23 mmol). After addition, the reaction mixture was slowly warmed to RT and stirred for 30 min. After reaction was completed, the reaction mixture was diluted with Sat. NH₄Cl. The precipitated solid was collected by filtration and washed with water to afford Compound 107 (ethyl 5-bromo-1,2-dimethyl-1H-indole-3-carboxylate) (1.13 g, 3.83 mmol, 92% yield). The product was used in next step without further purification.

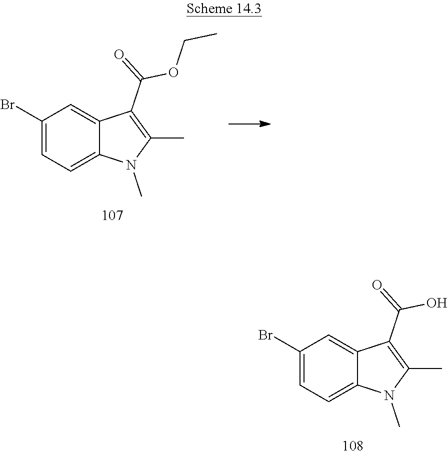 [SMILES: CCOC(=O)c1c(C)n(C)c2ccc(Br)cc12 ; Cc1c(C(=O)O)c2cc(Br)ccc2n1C]

To a solution of Compound 107 (1.13 g, 3.83 mmol) in MeOH/THF (40 mL, 3:1) was added 2N NaOH (10 mL). After addition, the reaction mixture was refluxed for overnight. After reaction was completed, the solvent was removed under reduced pressure, then acidified with 1N HCl. The precipitated solid was collected by filtration and washed with water to afford Compound 108 (5-bromo-1,2-dimethyl-1H-indole-3-carboxylic acid) (0.98 g, 3.65 mmol, 95% yield). The product was used in next step without further purification.

Compound 109 was synthesized by using the same methods as in Scheme 13.3, 13.4, Scheme 1.4, 1.5, and 1.8 with Compound 108.

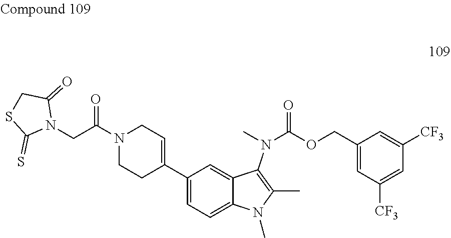 [SMILES: Cc1c(N(C)C(=O)OCc2cc(C(F)(F)F)cc(C(F)(F)F)c2)c2cc(C3=CCN(C(=O)CN4C(=O)CSC4=S)CC3)ccc2n1C]

Compound 109, ¹H-NMR (300 MHz, CDCl₃): 7.91-7.26 (m, 6H), 6.02-5.97 (d, 1H), 5.32-5.06 (m, 2H), 4.92-4.86 (d, 2H), 4.24 (s, 2H), 4.10 (s, 2H), 3.86-3.73 (m, 2H), 3.69 (s, 3H), 3.33 (s, 3H), 2.74-2.62 (m, 2H), 2.28 (s, 3H) ESI-MS m/z calcd for C₃₁H₂₈F₆N₄O₄S₂ 698.15, found 721.1[M+Na]⁺

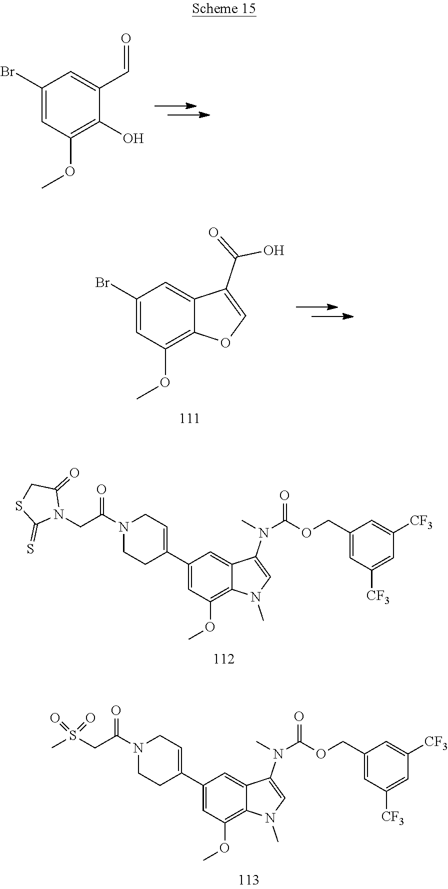 [SMILES: COc1cc(Br)cc(C=O)c1O ; COc1cc(Br)cc2c(C(=O)O)coc12 ; COc1cc(C2=CCN(C(=O)CN3C(=O)CSC3=S)CC2)cc2c(N(C)C(=O)OCc3cc(C(F)(F)F)cc(C(F)(F)F)c3)cn(C)c12 ; COc1cc(C2=CCN(C(=O)CS(C)(=O)=O)CC2)cc2c(N(C)C(=O)OCc3cc(C(F)(F)F)cc(C(F)(F)F)c3)cn(C)c12]

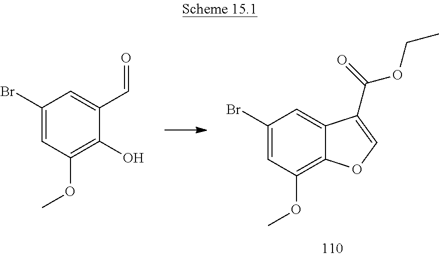 [SMILES: COc1cc(Br)cc(C=O)c1O ; CCOC(=O)c1coc2c(OC)cc(Br)cc12]

To a mixture of 5-Bromo-2-hydroxy-3-methoxybenzaldehyde (4.6 g, 19.91 mmol) and HBF₄.Et₂O (0.64 g, 2 mmol) in DCM (50 mL) was slowly added a solution of ethyl diazoacetate (3.6 g, 31.54 mmol) in DCM (20 mL) at 0° C. After addition, the reaction mixture was slowly warmed to RT and stirred for overnight. Then the reaction mixture was added conc. H₂SO₄ (2.6 g) and stirred for further 2 hours. After reaction was completed, the reaction mixture was neutralized with Na₂CO₃ and the solvent was removed under reduced pressure. The crude product was purified via flash column chromatography on a silica gel column using 10:1 Hexane-EtOAc as the eluent to give Compound 110 (ethyl 5-bromo-7-methoxybenzofuran-3-carboxylate) (1.9 g, 6.35 mmol, 32% yield).

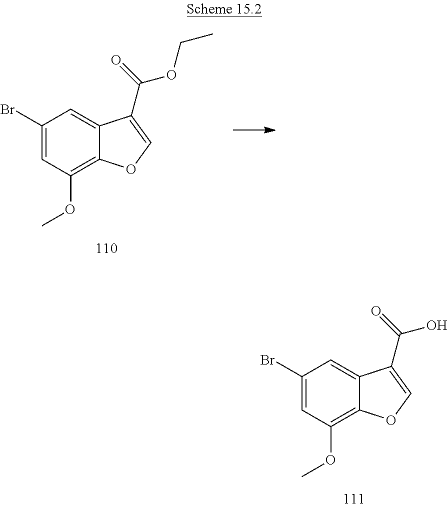 [SMILES: CCOC(=O)c1coc2c(OC)cc(Br)cc12 ; COc1cc(Br)cc2c(C(=O)O)coc12]

To a solution of Compound 110 (0.96 g, 3.21 mmol) in MeOH/THF (40 mL, 3:1) was added 2N NaOH (10 mL). After addition, the reaction mixture was refluxed for overnight. After reaction was completed, the solvent was removed under reduced pressure, then acidified with 1N HCl. The precipitated solid was collected by filtration and washed with water to afford Compound 111 (5-bromo-7-methoxybenzofuran-3-carboxylic acid) (0.80 g, 2.95 mmol, 92% yield). The product was used in next step without further purification.

Compound 112 was synthesized by using the same methods as in Scheme 13.3, 13.4, Scheme 1.4, 1.5, and 1.8 with Compound 111.

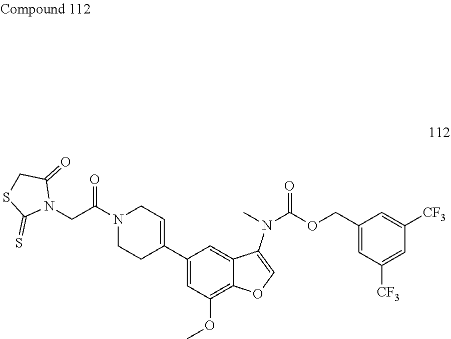 [SMILES: COc1cc(C2=CCN(C(=O)CN3C(=O)CSC3=S)CC2)cc2c(N(C)C(=O)OCc3cc(C(F)(F)F)cc(C(F)(F)F)c3)coc12]

Compound 112, ¹H-NMR (400 MHz, CDCl₃): 7.77-7.57 (m, 3H), 7.00-6.86 (m, 3H), 6.00-5.94 (d, 1H), 5.24 (s, 2H), 4.91-4.85 (d, 2H), 4.24 (s, 2H), 4.11 (s, 2H), 4.05 (s, 3H), 3.84-3.72 (m, 2H), 3.39 (s, 3H), 2.66-2.56 (m, 2H) ESI-MS m/z calcd for C₃₀H₂₅F₆N₃O₆S₂ 701.11, found 724.1 [M+Na]⁺

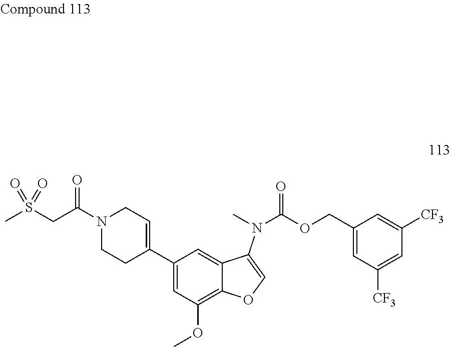 [SMILES: COc1cc(C2=CCN(C(=O)CS(C)(=O)=O)CC2)cc2c(N(C)C(=O)OCc3cc(C(F)(F)F)cc(C(F)(F)F)c3)coc12]

Compound 113, ¹H-NMR (400 MHz, CDCl₃): 7.90-7.27 (m, 6H), 6.00-5.95 (d, 1H), 5.36-5.20 (m, 2H), 4.30 (s, 2H), 4.18-4.14 (d, 2H), 3.91-3.81 (m, 2H), 3.32 (s, 3H), 3.15 (s, 3H), 2.68-2.60 (m, 2H), 2.35 (s, 3H) ESI-MS m/z calcd for C₂₈H₂₆F₆N₂O₆S 632.14, found 655.1 [M+Na]⁺

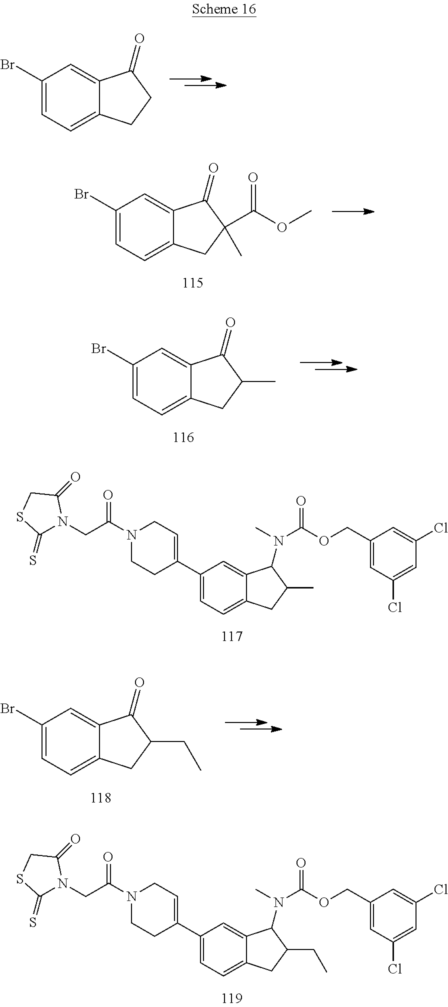 [SMILES: O=C1CCc2ccc(Br)cc21 ; COC(=O)C1(C)Cc2ccc(Br)cc2C1=O ; CC1Cc2ccc(Br)cc2C1=O ; CC1Cc2ccc(C3=CCN(C(=O)CN4C(=O)CSC4=S)CC3)cc2C1N(C)C(=O)OCc1cc(Cl)cc(Cl)c1 ; CCC1Cc2ccc(Br)cc2C1=O ; CCC1Cc2ccc(C3=CCN(C(=O)CN4C(=O)CSC4=S)CC3)cc2C1N(C)C(=O)OCc1cc(Cl)cc(Cl)c1]

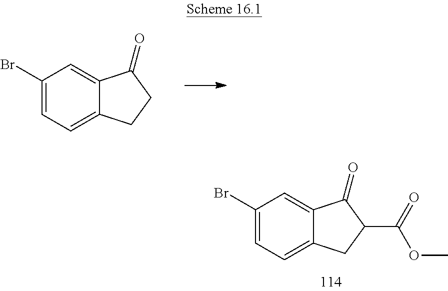

To the anhydrous dimethyl carbonate (50 mL) was added NaH (2.2 g), and then to the reaction mixture was added a solution of 6-Bromo-1-indanone (3.0 g, 14.21 mmol) in dimethyl carbonate (10 mL). After addition, the reaction mixture was added DMF (1 mL) and then heated at 80° C. for overnight. After reaction was completed, the dimethyl carbonate was removed under reduced. pressure. The residual was diluted with water and the precipitated solid was collected by filtration to afford Compound 114 (methyl 6-bromo-1-oxo-2,3-dihydro-1H-indene-2-carboxylate) (2.7 g, 10.03 mmol, yield 71%). The product was used in next step without further purification.

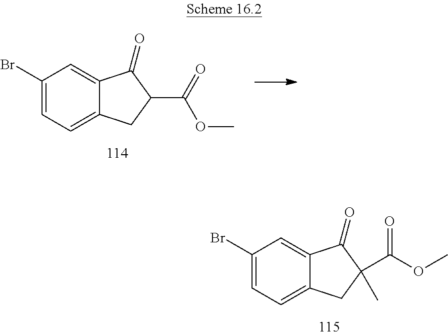 [SMILES: COC(=O)C1Cc2ccc(Br)cc2C1=O ; COC(=O)C1(C)Cc2ccc(Br)cc2C1=O]

To a solution of Compound 114 (2.7 g, 10.03 mmol) in DMSO (50 mL) was added K₂CO₃ (2.8 g, 20.26 mmol) and MeI (3.0 g, 21.14 mmol). After addition, the reaction mixture was stirred for overnight. After reaction was completed, the reaction was diluted with water and the precipitated solid was collected by filtration to afford Compound 115 (methyl 6-bromo-2-methyl-1-oxo-2,3-dihydro-1H-indene-2-carboxylate) (2.6 g, 9.18 mmol, yield 91%). The product was used in next step without further purification.

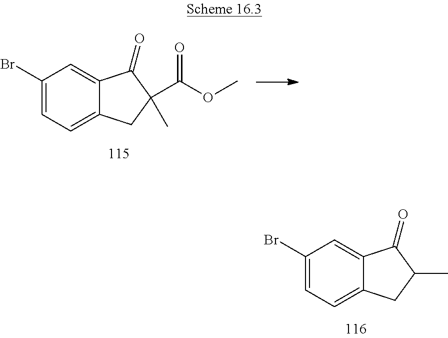 [SMILES: COC(=O)C1(C)Cc2ccc(Br)cc2C1=O ; CC1Cc2ccc(Br)cc2C1=O]

Compound 115 (2.6 g, 9.18 mmol) was dissolved in a mixture of cone. HCl (10 mL) and AcOH (30 mL) and heated at 65° C. for 5 hours. After reaction was completed, the dimethyl carbonate was removed under reduced. pressure. The residual was quenched with Sat. NaHCO₃ and extracted with EtOAc. The combined organic layers were washed with brine and dried over MgSO₄ and concentrated in vacuo to afford Compound 116 (6-bromo-2-methyl-2,3-dihydro-1H-inden-1-one) (1.55 g, 6.89 mmol, yield 75%). The product was used in next step without further purification.

Compound 117 was synthesized by using the same methods as in Scheme 1.1, 1.2, 1.3, 1.4, 1.5, and 1.8 with Compound 116.

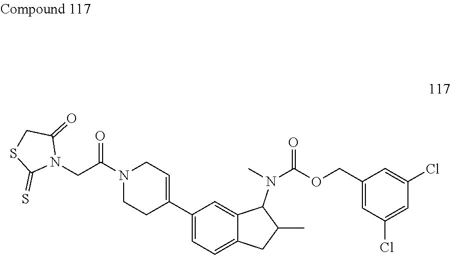 [SMILES: CC1Cc2ccc(C3=CCN(C(=O)CN4C(=O)CSC4=S)CC3)cc2C1N(C)C(=O)OCc1cc(Cl)cc(Cl)c1]

Compound 117, ¹H-NMR (500 MHz, CDCl₃): 7.31-7.05 (m, 6H), 6.06-6.02 (d, 1H), 5.69-5.40 (m, 1H), 5.18-5.14 (m, 2H), 4.90-4.84 (d, 2H), 4.23 (s, 2H), 4.10 (s, 2H), 3.82-3.72 (m, 2H), 3.11-3.03 (m, 1H), 2.79-2.57 (m, 3H), 2.52 (s, 3H), 1.28-1.08 (m, 4H) ESI-MS m/z calcd for C₂₉H₂₉Cl₂N₃O₄S₂ 617.10, found 640.1 [M+Na]⁺

Compound 118 was synthesized by using the same methods as in Scheme 16.1, 16.2, 16.3 with ethyl iodide. With similar methods for the synthesis of Compound 117, Compound 119 was synthesized by using the precursor Compound 118.

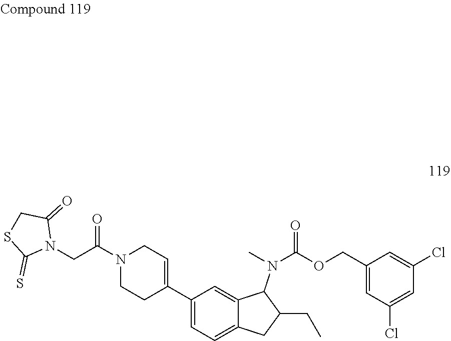 [SMILES: CCC1Cc2ccc(C3=CCN(C(=O)CN4C(=O)CSC4=S)CC3)cc2C1N(C)C(=O)OCc1cc(Cl)cc(Cl)c1]

Compound 119, ¹H-NMR (400 MHz, CDCl₃): 7.31-7.25 (m, 6H), 6.07-6.02 (d, 1H), 5.73-5.62 (m, 1H), 5.20-5.09 (m, 2H), 4.91-4.84 (d, 2H), 4.23 (s, 2H), 4.11 (s, 2H), 3.85-3.74 (m, 2H), 3.12-3.06 (m, 1H), 2.74-2.47 (m, 7H), 1.37-1.24 (m, 2H), 1.04-0.98 (m, 3H) ESI-MS m/z calcd for C₃₀H₃₁Cl₂N₃O₄S₂ 631.11, found 654.4 [M+Na]⁺

Compound 120 and 121 are synthesized by using the same methods as the synthesis for Compound 119 with the corresponding precursors for Compound 18 and 22, respectively.

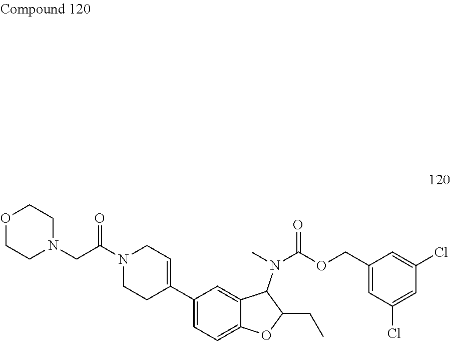 [SMILES: CCC1Oc2ccc(C3=CCN(C(=O)CN4CCOCC4)CC3)cc2C1N(C)C(=O)OCc1cc(Cl)cc(Cl)c1]

Compound 120, ¹H-NMR (300 MHz, CDCl₃): 7.37-7.16 (m, 6H), 6.08-6.02 (d, 1H), 5.74-5.61 (m, 1H), 5.21-5.08 (m, 2H), 4.26-4.21 (m, 2H), 3.87-3.73 (m, 6H), 3.26-3.23 (m, 2H), 3.12-3.04 (m, 1H), 2.74-2.46 (m, 11H), 1.38-1.24 (m, 2H), 1.05-1.00 (m, 3H) ESI-MS m/z calcd for C₃₁H₃₇Cl₂N₃O₄ 585.22, found 586.6[M+H]⁺

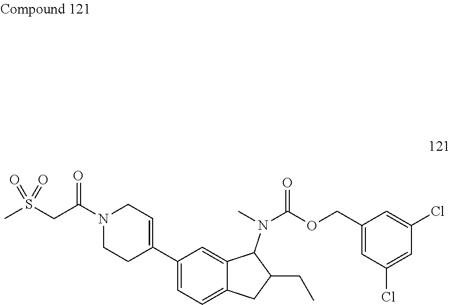 [SMILES: CCC1Cc2ccc(C3=CCN(C(=O)CS(C)(=O)=O)CC3)cc2C1N(C)C(=O)OCc1cc(Cl)cc(Cl)c1]

Compound 121, ¹H-NMR (400 MHz, CDCl₃): 7.33-7.17 (m, 6H), 6.06-6.01 (d, 1H), 5.74-5.61 (m, 1H), 5.23-5.08 (m, 2H), 4.29 (s, 2H), 4.18-4.13 (m, 3H), 3.90-3.75 (m, 2H), 3.17-3.05 (m, 4H), 2.70-2.45 (m, 6H), 1.34-1.24 (m, 2H), 1.04-0.97 (m, 3H) ESI-MS m/z calcd for C₂₈H₃₂Cl₂N₂O₅S 578.14, found 601.5 [M+Na]⁺

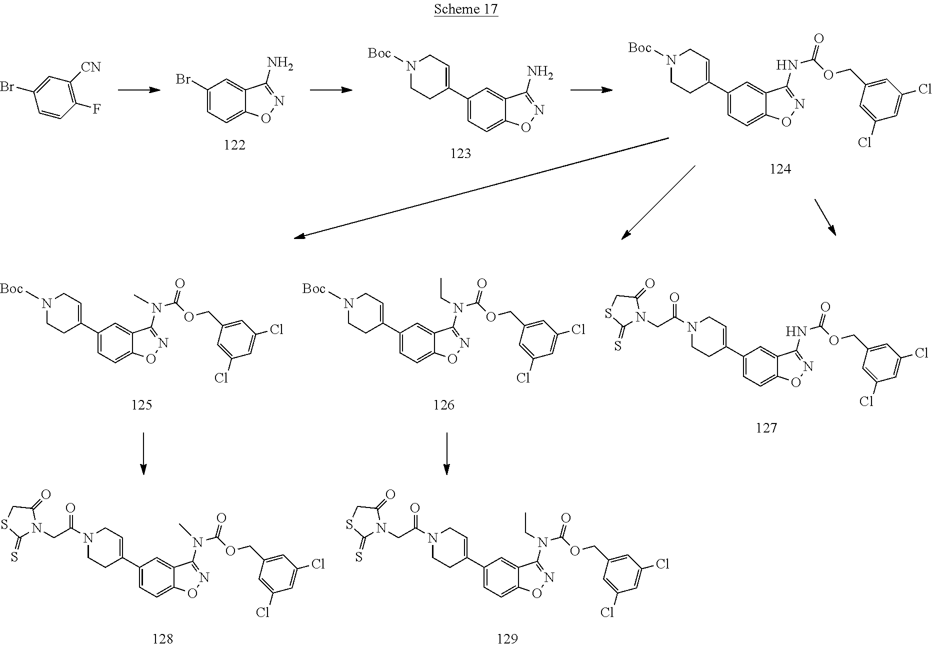

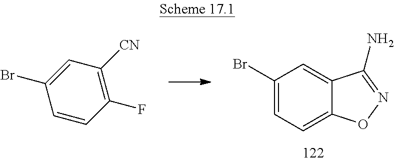 [SMILES: N#Cc1cc(Br)ccc1F ; Nc1noc2ccc(Br)cc12]

N-hydroxyacetamide (2.63 g, 35.0 mmol) was dissolved in DMF (100 mL), and then t-BuOK (3.93 g, 35.0 mmol) was added in one portion. The temperature rose to 30° C. The mixture was stirred for 1 h, and 5-bromo-2-fluorobenzonitrile (7 g, 35.0 mmol) was added. The reaction mixture was stirred for overnight. An additional portion of t-BuOK (1.96 g, 17.5 mmo) was added and the reaction was allowed to stir overnight. The mixture was poured into brine and CH₂Cl₂ and the layers were separated. The organic phase was dried over MgSO₄ and concentrated in vacuo. The residue was purified by flash column chromatography using EtOAc/Hexane (1/2) to afford the Compound 122 (5-Bromobenzo[d]isoxazo-3-amine) (4.59 g, 62%) as a white-off solid.

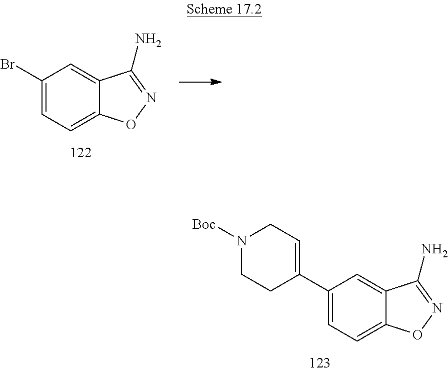 [SMILES: Nc1noc2ccc(Br)cc12 ; CC(C)(C)OC(=O)N1CC=C(c2ccc3onc(N)c3c2)CC1]

A mixture solution of 5-Bromobenzo[d]isoxazo3-amine (1 g, 4.694 mmol) and boronic acid (2.9 g, 9.388 mmol) in degassed dioxane (30 mL) was stirred at room temperature under Ar atmosphere, followed by the addition of 2M Na₂CO_{3(aq)} (7 mL, 14.08 mmol) and Pd(dppf)Cl₂ (175 mg, 0.235 mmol). The reaction mixture was stirred at 80° C. overnight. The solution was filtered through Celite, dried over MgSO₄ and concentrated in vacuo. Purification on silica gel column chromatography using ethyl acetate/hexane=1/2 as elution to yield the desired product of Compound 123 (1.03 g, 70%) as a white solid. ¹H NMR (300 MHz, CD₃OD) δ 7.79 (s, 1H), 7.67 (d, 1H, J=6.9 Hz), 7.35 (d, 1H, J=6.9 Hz), 6.13 (s, 1H), 4.09 (br. s, 2H), 3.67 (br. s, 2H), 2.59 (br. s, 2H), 1.51 (s, 9H). ESI-MS m/z calcd for C₁₇H₂₁N₃O₃ 315.158, found 316.1 [M+H]⁺.

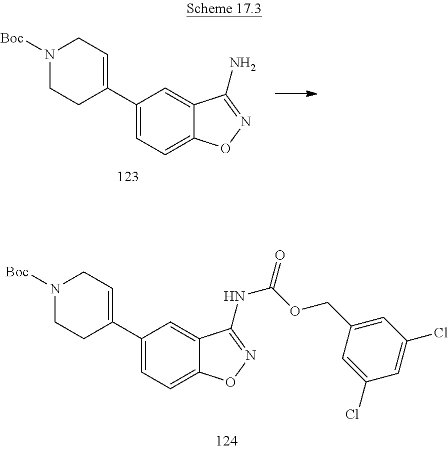 [SMILES: CC(C)(C)OC(=O)N1CC=C(c2ccc3onc(N)c3c2)CC1 ; CC(C)(C)OC(=O)N1CC=C(c2ccc3onc(NC(=O)OCc4cc(Cl)cc(Cl)c4)c3c2)CC1]

A mixture solution of Compound 123 (430 mg, 1.363 mmol), triphosgene (405 mg, 1.363 mmol) and Et₃N (0.57 mL, 4.09 mmol) in THF was stirred at 0° C., and then the reaction was warmed to room temperature. After stirring for 2 hours, a solution of 3,5-dichlorobenzyl alcohol (266 mg, 1.5 mmol) and Et₃N (0.57 mL, 4.09 mmol) in THF was added to the reaction solution and was stirred under reflux overnight. The reaction solution was diluted with ethyl acetate, washed with sat. NaHCO_{3(aq)}, dried over MaSO₄ and concentrated in vacuo. The residue was purified by flash column chromatography using EtOAc/Hexane (1/5) to afford the desired product of Compound 124 (360 mg, 51%) as a white solid. ¹H NMR (300 MHz, CDCl₃) δ 8.17 (s, 1H, NH), 8.08 (s, 1H), 7.62 (dd, 1H, J=1.8, 8.7 Hz), 7.42 (d, 1H, J=8.7 Hz), 7.36-7.33 (m, 3H), 6.04 (br. s, 1H), 5.25 (s, 2H), 4.10-4.09 (m, 2H), 3.67 (t, 2H, J=5.7 Hz), 2.56 (br. s, 2H), 1.50 (s, 9H). ESI-MS m/z calcd for C₂₅H₂₅Cl₂N₃O₅ 517.117, found 518.1 [M+H]⁺.

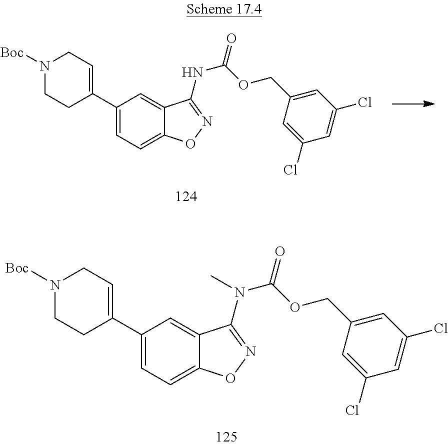 [SMILES: CC(C)(C)OC(=O)N1CC=C(c2ccc3onc(NC(=O)OCc4cc(Cl)cc(Cl)c4)c3c2)CC1 ; CN(C(=O)OCc1cc(Cl)cc(Cl)c1)c1noc2ccc(C3=CCN(C(=O)OC(C)(C)C)CC3)cc12]

Compound 124 (100 mg, 0.193 mmol) was dissolved in DMF (2 mL), and then NaH (10 mg, 0.251 mmol) was added at 0° C. After stirring at 0° C. for 15 mins, MeI (0.014 mL, 0.231 mmol) was added to the reaction solution at 0° C. The reaction was allowed to warm to room temperature. After stirring at room temperature for 1 h, the reaction solution was diluted with ethyl acetate, washed with sat. NH₄Cl_{(aq)}, and the aqueous phase was extracted with ethyl acetate. The combined organic phase was dried over MgSO₄ and concentrated under reduced pressure. Purification on silica gel column chromatography using ethyl acetate/hexane=1/5 as elution to yield the desired product of Compound 125 (80 mg, 78% steps) as a colorless oil. ¹H NMR (300 MHz, CDCl₃) δ 7.58 (dd, 1H, J=0.9, 6.6 Hz), 7.53 (s, 1H), 7.48 (d, 1H, J=6.6 Hz), 7.33 (s, 1H), 7.22 (s, 2H), 5.93 (br. s, 1H), 5.21 (s, 2H), 4.07 (br. s, 2H), 3.63 (t, 2H, J=4.2 Hz), 3.56 (s, 3H), 2.44 (br.s, 2H), 1.51 (s, 9H). ESI-MS m/z calcd for C₂₆H₂₇Cl₂N₃O₅ 531.133, found 532.1 [M+H]⁺

Compound 126 was synthesized by using the same method in Scheme 17.4 with ethyl iodide.

Compound 127, 128, 129 are synthesized by using the same methods as in Scheme 1.5, and 1.8 with Compound 124, 125, 126, respectively.

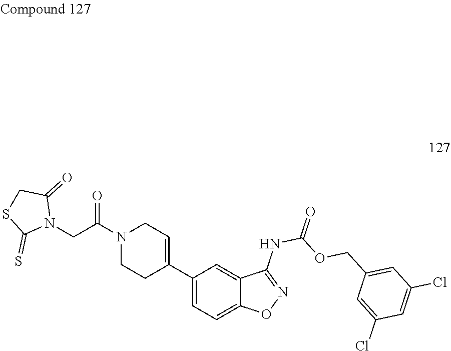 [SMILES: O=C(Nc1noc2ccc(C3=CCN(C(=O)CN4C(=O)CSC4=S)CC3)cc12)OCc1cc(Cl)cc(Cl)c1]

Compound 127, ¹H NMR (400 MHz, CDCl₃) δ 8.55 (br s, 1H, NH), 8.12 (s, 1H), 7.61 (d, 1H, J=11.6 Hz), 7.49 (d, 1H, J=11.6 Hz), 7.34 (s, 2H), 7.27 (s, 1H), 6.05 (d, 1H, J=14.4 Hz), 5.24 (s, 2H), 4.88 (d, 2H, J=21.2 Hz), 4.25 (d, 2H, J=3.2 Hz), 4.09 (s, 2H), 3.80 (dt, 2H, J=7.2, 32 Hz), 2.66 (d, 2H, J=55.6 Hz). ESI-MS m/z calcd for C₂₅H₂₀Cl₂N₄O₅S₂ 590.025, found 613 [M+Na]⁺.

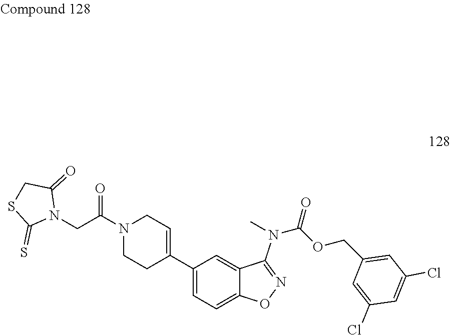 [SMILES: CN(C(=O)OCc1cc(Cl)cc(Cl)c1)c1noc2ccc(C3=CCN(C(=O)CN4C(=O)CSC4=S)CC3)cc12]

Compound 128, ¹H NMR (400 MHz, CDCl₃) δ 7.58-7.52 (m, 2H), 7.35-7.23 (m, 4H), 5.96 (d, 1H, J=19.6 Hz), 5.22 (s, 2H), 4.89 (d, 2H, J=23.6 Hz), 4.24 (s, 2H), 4.11 (s, 2H), 3.78 (d, 2H, J=33.2 Hz), 3.58 (s, 3H), 2.56 (d, 2H, J=43.6 Hz). ESI-MS m/z calcd for C₂₆H₂₂Cl₂N₄O₅S₂ 604.041, found 627 [M+Na]⁺.

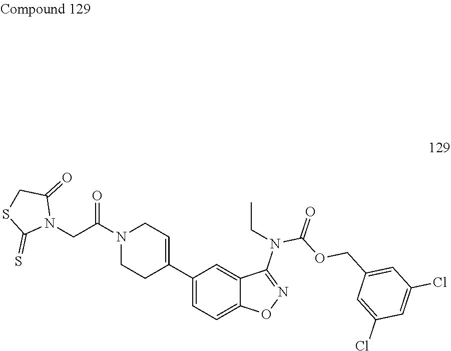 [SMILES: CCN(C(=O)OCc1cc(Cl)cc(Cl)c1)c1noc2ccc(C3=CCN(C(=O)CN4C(=O)CSC4=S)CC3)cc12]

Compound 129, ¹H NMR (400 MHz, CDCl₃) δ 7.60-7.46 (m, 3H), 7.30 (d, 1H, J=22.8 Hz), 7.18 (s, 2H), 5.95 (d, 1H, J=20 Hz), 5.20 (s, 2H), 4.88 (d, 2H, J=24 Hz), 4.23 (s, 2H), 4.11 (s, 2H), 4.00 (q, 2H, J=6.8 Hz), 3.78 (dt, 2H, J=5.6, 33.2 Hz), 2.55 (d, 2H, J=44.8 Hz), 1.36 (t, 3H, J=6.8 Hz). ESI-MS m/z calcd for C₂₇H₂₄Cl₂N₄O₅S₂ 618.041, found 641 [M+Na]⁺.

Compound 130 was synthesized by using the same methods for Compound 9 by replacing methyl amine with ethyl amine in Scheme 1.1.

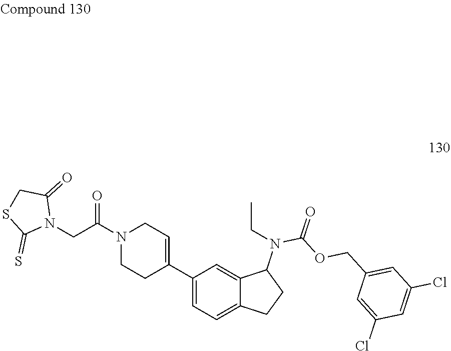 [SMILES: CCN(C(=O)OCc1cc(Cl)cc(Cl)c1)C1CCc2ccc(C3=CCN(C(=O)CN4C(=O)CSC4=S)CC3)cc21]

Compound 130, ¹H NMR (400 MHz, CDCl₃) δ 7.32-7.07 (m, 6H), 6.01-5.94 (m, 1H), 5.80-5.46 (m, 1H), 5.17-5.03 (m, 2H), 4.88 (d, 2H, J=23.2 Hz), 4.22 (s, 2H), 4.10 (s, 2H), 3.84-3.72 (m, 2H), 3.33-3.17 (m, 2H), 3.05-2.89 (m, 2H), 2.87-2.81 (m, 1H), 2.66-2.60 (m, 1H), 2.53-2.47 (m, 2H), 2.17-1.99 (m, 1H), 1.11-1.17 (m, 2H). ESI-MS m/z calcd for C₂₉H₂₉Cl₂N₃O₄S₂ 617.098, found 618.1 [M+H]⁺.

Compound 131 was synthesized by using the same methods for Compound 127 with precursor Compound 89.

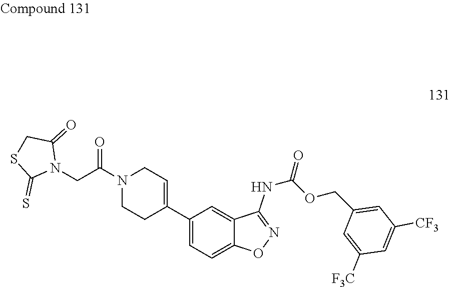 [SMILES: O=C(Nc1noc2ccc(C3=CCN(C(=O)CN4C(=O)CSC4=S)CC3)cc12)OCc1cc(C(F)(F)F)cc(C(F)(F)F)c1]

Compound 131, ¹H NMR (400 MHz, CDCl₃) δ 8.68 (br s, 1H, NH), 8.14 (s, 1H), 7.94 (s, 2H), 7.89 (s, 1H), 7.63 (d, 1H, J=8.8 Hz), 7.49 (d, 1H, J=8.8 Hz), 6.07 (d, 1H, J=14.8 Hz), 5.42 (s, 2H), 4.88 (d, 2H, J=22.4 Hz), 4.26 (s, 2H), 4.10 (s, 2H), 3.81 (dt, 2H, J=5.6, 31.2 Hz), 2.68 (d, 2H, J=54.4 Hz). ESI-MS m/z calcd for C₂₇H₂₀F₆N₄O₅S₂ 658.078, found 659.1 [M+H]⁺.

Compound 132 and 133 are synthesized by using the same methods for Compound 128 and 129, respectively, with precursor Compound 89.

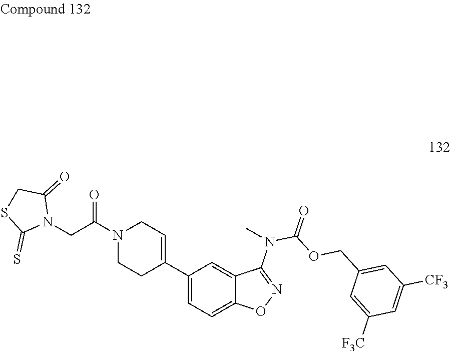 [SMILES: CN(C(=O)OCc1cc(C(F)(F)F)cc(C(F)(F)F)c1)c1noc2ccc(C3=CCN(C(=O)CN4C(=O)CSC4=S)CC3)cc12]

Compound 132, ¹H NMR (400 MHz, CDCl₃) δ 7.86-7.79 (m, 3H), 7.65-7.51 (m, 3H), 5.98 (d, 1H, J=20.0 Hz), 5.40 (s, 2H), 4.87 (d, 2H, J=23.2 Hz), 4.23 (s, 2H), 4.11 (s, 2H), 3.78 (dt, 2H, J=5.6, 33.2 Hz), 3.59 (s, 3H), 2.58 (d, 2H, J=46.4 Hz). ESI-MS m/z calcd for C₂₈H₂₂F₆N₄O₅S₂ 672.094, found 673.1 [M+H]⁺.

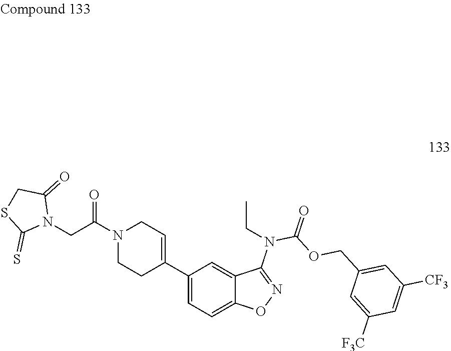 [SMILES: CCN(C(=O)OCc1cc(C(F)(F)F)cc(C(F)(F)F)c1)c1noc2ccc(C3=CCN(C(=O)CN4C(=O)CSC4=S)CC3)cc12]

Compound 133, ¹H NMR (400 MHz, CDCl₃) δ 7.83-7.73 (m, 3H), 7.62-7.54 (m, 3H), 5.97 (d, 1H, J=21.2 Hz), 5.37 (s, 2H), 4.87 (d, 2H, J=23.2 Hz), 4.23 (s, 2H), 4.11 (s, 2H), 4.00 (q, 2H, J=6.8 Hz), 3.77 (dt, 2H, J=5.6, 33.6 Hz), 2.57 (d, 2H, J=45.6 Hz), 1.37 (t, 3H, J=6.8 Hz). ESI-MS m/z calcd for C₂₉H₂₄F₆N₄O₅S₂ 686.109, found 687.1 [M+H]⁺.

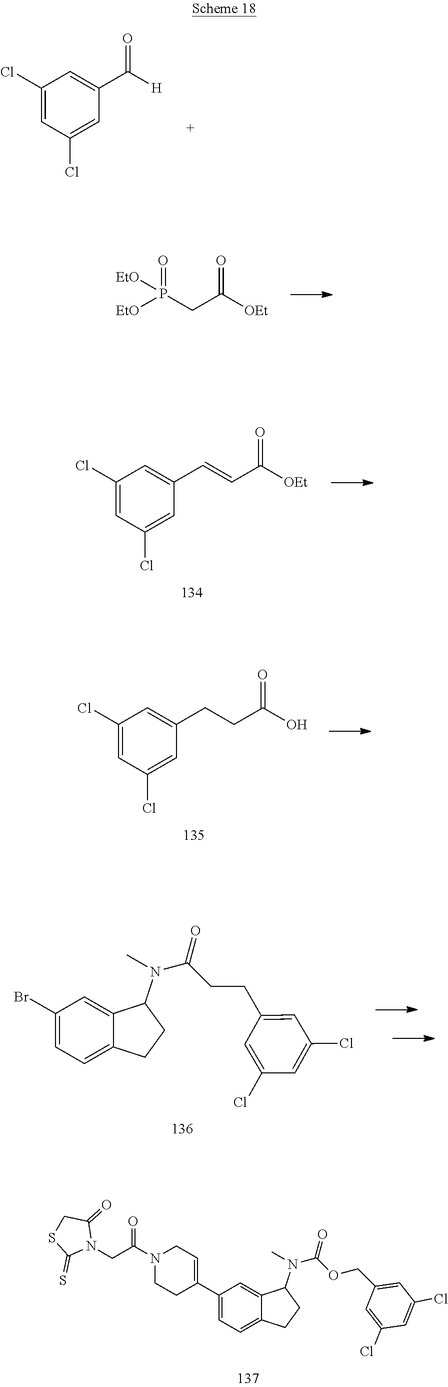 [SMILES: O=Cc1cc(Cl)cc(Cl)c1 ; CCOC(=O)CP(=O)(OCC)OCC ; CCOC(=O)/C=C/c1cc(Cl)cc(Cl)c1 ; O=C(O)CCc1cc(Cl)cc(Cl)c1 ; CN(C(=O)CCc1cc(Cl)cc(Cl)c1)C1CCc2ccc(Br)cc21 ; CN(C(=O)OCc1cc(Cl)cc(Cl)c1)C1CCc2ccc(C3=CCN(C(=O)CN4C(=O)CSC4=S)CC3)cc21]

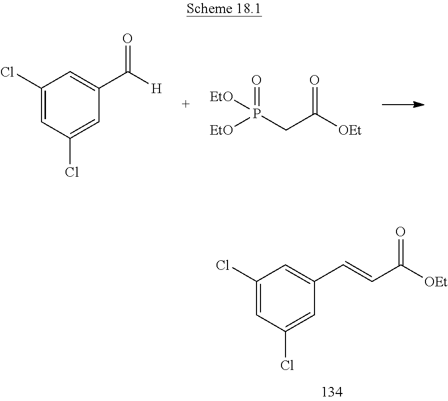 [SMILES: O=Cc1cc(Cl)cc(Cl)c1 ; CCOC(=O)CP(=O)(OCC)OCC ; CCOC(=O)/C=C/c1cc(Cl)cc(Cl)c1]

To a solution of triethyl phosphonoacetate (0.82 mL, 4.13 mmol) in dried THF was added NaH (330 mg, 8.26 mmol) slowly at 0° C. under nitrogen atmosphere. After stirring at 0° C. for 30 mins, 3,5-dichlorobenzaldehyde (723 mg, 4.13 mmol) was added to the reaction solution and warmed to room temperature. After stirring for 2 hours, H₂O was added to the reaction solution and extracted with ethyl acetate. After evaporation, the crude product was purified by silica gel column chromatography using ethyl acetate/Hexane=1/10 as elution to yield the ester product of Compound 134 (800 mg, 80%) as a white solid.

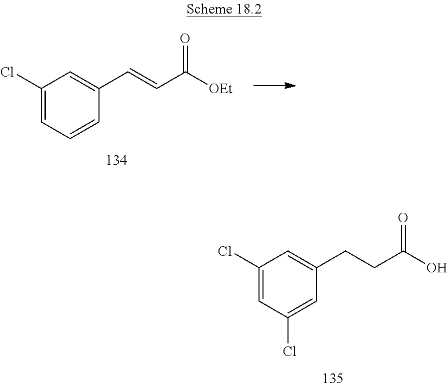 [SMILES: CCOC(=O)/C=C/c1cccc(Cl)c1 ; O=C(O)CCc1cc(Cl)cc(Cl)c1]

A mixture solution of Compound 134 and 5% Pd/C in methanol was stirred at room temperature under H₂ atmosphere overnight. After filtration and concentration, the residue was dissolved in MeOH, followed by the addition of 1N NaOH_{(aq)}. The mixture solution was stirred at room temperature overnight. The reaction was quenched with 1N HCl_{(aq)}. After evaporation, the crude product of Compound 135 was used in next step without further purification.

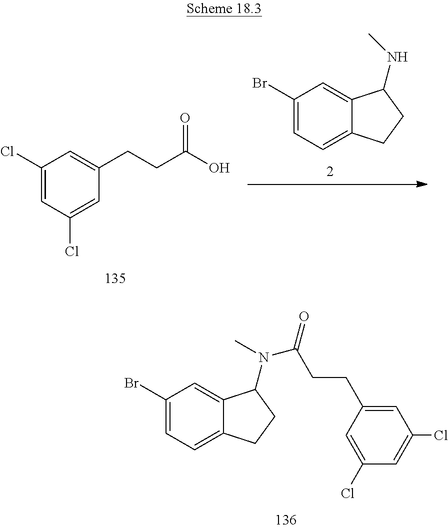 [SMILES: O=C(O)CCc1cc(Cl)cc(Cl)c1 ; CNC1CCc2ccc(Br)cc21 ; CN(C(=O)CCc1cc(Cl)cc(Cl)c1)C1CCc2ccc(Br)cc21]

A mixture solution of Compound 2 (150 mg, 0.664 mmol), EDCI (192 mg, 0.996 mmol), HOBt (52 mg, 0.332 mmol) and NMM (0.2 mL, 1.996 mmol) and Compound 135 (250 mg, 0.968 mmol) in DCM was stirred at room temperature overnight. The reaction solution was diluted with DCM, washed with sat. NH₄Cl_{(aq)}, dried over Na₂SO₄ and concentrated in vacuo. The residue was purified by flash column chromatography using EtOAc/Hexane (1/1) to afford the desired product of Compound 136 (360 mg, 51%).

Compound 137 was synthesized by using the same methods as in Scheme 1.4, 1.5, and 1.8 with Compound 136.

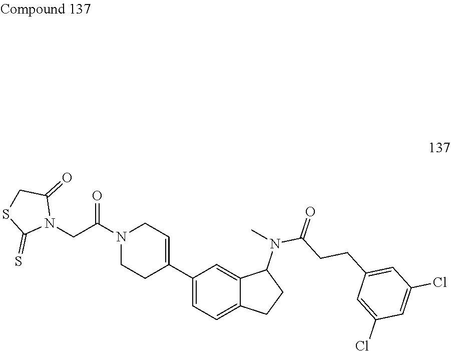 [SMILES: CN(C(=O)CCc1cc(Cl)cc(Cl)c1)C1CCc2ccc(C3=CCN(C(=O)CN4C(=O)CSC4=S)CC3)cc21]

Compound 137, ¹H NMR (400 MHz, CDCl₃) δ 7.25-7.20 (m, 3H), 7.19-7.17 (m, 2H), 7.10-7.01 (m, 1H), 6.05-5.95 (m, 1H), 5.84 (d, 2H), 4.29-4.20 (m, 2H), 3.86-3.65 (m, 1H), 2.97-2.60 (m, 8H), 2.58-2.32 (m, 3H), 2.08-1.98 (m, 2H). ESI-MS m/z calcd for C₂₉H₂₉Cl₂N₃O₃S₂ 601.10, found 624.1 [M+Na]⁺.

Compound 138 was synthesized by using the same methods for Compound 22 with the corresponding precursor 4-oxo-2-thioxo-3-thiazolidinylacetic acid.

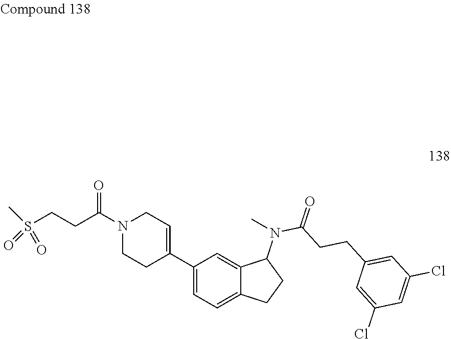 [SMILES: CN(C(=O)CCc1cc(Cl)cc(Cl)c1)C1CCc2ccc(C3=CCN(C(=O)CCS(C)(=O)=O)CC3)cc21]

Compound 138, ¹H NMR (400 MHz, CDCl₃) δ 7.38-7.04 (m, 6H), 6.04-5.98 (m, 1H), 5.91-5.87 (m, 1H), 5.22-5.10 (m, 2H), 4.21-4.24 (m, 1H), 4.10-4.15 (m, 1H), 3.80-3.84 (m, 1H), 3.69 (t, 1H, J=6 Hz), 3.48 (t, 2H, J=6.8 Hz), 2.82-3.03 (m, 7H), 2.69-2.22 (m, 6H), 2.05-1.95 (m, 1H). ESI-MS m/z calcd for C₂₇H₃₀Cl₂N₂O₅S 564.13, found 587.1 [M+Na]⁺.

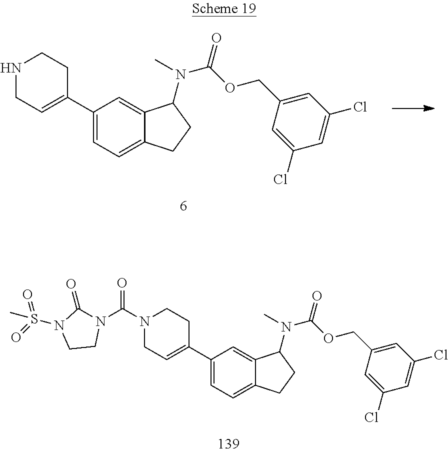 [SMILES: CN(C(=O)OCc1cc(Cl)cc(Cl)c1)C1CCc2ccc(C3=CCNCC3)cc21 ; CN(C(=O)OCc1cc(Cl)cc(Cl)c1)C1CCc2ccc(C3=CCN(C(=O)N4CCN(S(C)(=O)=O)C4=O)CC3)cc21]

A solution of Compound 6 (195 mg, 0.452 mmol) in dried CH₂Cl₂ (10 mL) was stirred at 0° C., followed by addition of Et₃N (0.13 mL, 0.942 mmol) and 3-chlorocarbonyl-1-methanesulfonyl-2-imidazolidinone (157 mg, 0.678 mmol). The reaction mixture was stirred at room temperature overnight and monitored by TLC. The mixture was added water and extracted with CH₂Cl₂. The organic phase was dried with MgSO₄ and concentrated under reduced pressure. The crude product was purified by silica gel column chromatography using CH₂Cl₂/ethyl acetate=3/1 as elution to yield the desired product of Compound 139 (97 mg, 35%) as a yellow oil.

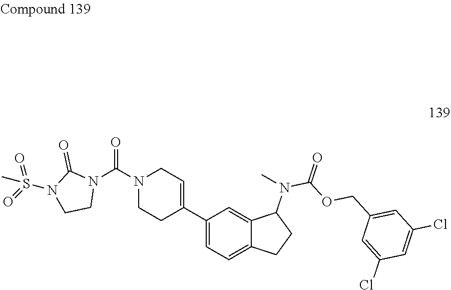 [SMILES: CN(C(=O)OCc1cc(Cl)cc(Cl)c1)C1CCc2ccc(C3=CCN(C(=O)N4CCN(S(C)(=O)=O)C4=O)CC3)cc21]

Compound 139, ¹H NMR (400 MHz, CDCl₃) δ 7.37-7.20 (m, 6H), 7.14-7.11 (m, 1H), 5.94-6.02 (m, 1H), 5.90-5.70 (m, 1H), 5.10-5.24 (m, 2H), 4.19-4.4.15 (m, 2H), 3.99-3.72 (m, 4H), 3.60-3.80 (m, 2H), 3.34 (s, 3H), 3.03-2.98 (m, 1H), 2.96-2.84 (m, 1H), 2.69-2.66 (m, 4H), 2.50-2.35 (m, 1H), 2.04-1.96 (m, 1H). ESI-MS m/z calcd for C₂₈H₃₀Cl₂N₄O₆S 620.13, found 643.1 [M+Na]⁺.

### Example II. Autotaxin Inhibitor Screening Assay

Autotaxin activity was measured by choline release from LPC in presence or absence of compound.

Twenty (20) ng autotaxin (10803, Cayman, MI, USA) was incubated with 100 μM 14:0 LPC (855575P, Avanti, AL, USA) in a final volume of 100 μL buffer containing 50 mM Tris pH 8.0, 0.01% Triton X-100, 50 mM CaCl₂, 1 unit/ml choline oxidase, 2 unit/ml HRP, 2 mM homovanilic acid (HVA). The relative amount of released choline was measured by HVA fluorescence in a 96-well plate. Fluorescent intensity was determined at λex/λem=320/450 nm every 60 seconds for 90 minutes with a SpectraMax i3 (Molecular Devices, CA, USA). Data analysis was performed using GraphPad Prism (GraphPad, La Jolla Calif., USA).

Inhibition %=[1−Slope_{TA}−Slope_{Blank})/(Slope_{vehicle}−Slope_{Blank})]×100%

The inhibition rates of the compounds of the present disclosure on the activity of autotaxin enzyme are shown in Table 1.

TABLE 1

| Compound | |
|---|---|
| Number | Inhibition |
| 8 | B |
| 9 | A |
| 10 | B |
| 11 | B |
| 12 | B |
| 13 | C |
| 14 | C |
| 15 | C |
| 16 | B |
| 17 | B |
| 18 | A |
| 19 | C |
| 20 | B |
| 21 | B |
| 22 | A |
| 23 | B |
| 24 | C |
| 25 | A |
| 26 | C |
| 27 | A |
| 28 | B |
| 29 | A |
| 30 | A |
| 33 | B |
| 34 | B |
| 36 | A |
| 37 | B |
| 38 | C |
| 43 | C |
| 44 | C |
| 50 | C |
| 51 | C |
| 52 | C |
| 53 | C |
| 54 | C |
| 58 | C |
| 59 | C |
| 60 | C |
| 61 | C |
| 66 | A |
| 69 | A |
| 73 | C |
| 74 | C |
| 82 | C |
| 85 | A |
| 92 | A |
| 93 | A |
| 94 | A |
| 95 | A |
| 96 | A |
| 97 | A |
| 102 | A |
| 103 | B |
| 105 | A |
| 109 | B |
| 112 | A |
| 113 | B |
| 117 | A |
| 119 | B |
| 120 | C |
| 121 | C |
| 127 | B |
| 128 | B |
| 129 | B |
| 130 | A |
| 131 | B |
| 132 | B |
| 133 | B |
| 137 | B |
| 138 | B |
| 139 | C |

A: above 80% inhibition at 1 μM;
B: 80% to 50% inhibition at 1 μM;
C: below 50% inhibition at 1 μM.

It will be apparent to those skilled in the art that various modifications and variations can be made to the disclosed embodiments. It is intended that the specification and examples be considered as exemplary only, with the true scope of the disclosure being indicated by the following claims and their equivalents.

1. A benzene fused heterocyclic compound of Formula (I), or a pharmaceutically acceptable salt, solvate, hydrate, geometric isomers, enantiomer, diastereoisomer or racemate thereof: 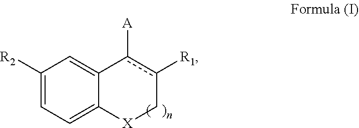
wherein [symbol] is a single or double bond;
n is 0 or 1;
X is —CH₂—, O, NR₁, or S;
A is —C(Rₐ₁)(Rₐ₂)(Rₐ₃) or —N(Rₐ₁)(Rₐ₂),
wherein Rₐ₁, Rₐ₂ and Rₐ₃ are independently selected from a group consisting of:
H, alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂ RₐₐR_{bb} and —SO₂ NRₐₐR_{bb} which are optionally substituted by at least one substituent independently selected from a group consisting of:
alkyl, cycloalkyl, heterocyclic alkyl, aryl, —Y_{bb}, —Ar_{bb}Y_{bb}, —OR_{cc}, and —OAr_{bb}Y_{bb},
wherein Rₐₐ, R_{bb} and R_{cc} independently are nil, H, halogen, alkyl, or aryl, Y_{bb} is CN or halogen, and Arₐₐ and Ar_{bb} independently are aryl or heteroaryl;
R₁ is H or alkyl;
R₂ is alkyl, cycloalkyl, heterocylic alkyl, aryl, heteroaryl, C₁-C₆ hydrocarbon optionally substituted by at least one substituent independently selected from a group consisting of:
R₂ₐOR_{2b}, —R₂ₐC(O)OR_{2b}R_{2c}, —R₂ₐC(O)R_{2b}R_{2c}, —R₂ₐC(O)NR_{2b}R_{2c}, —R₂ₐNR_{2b}C(O)NR_{2c}R_{2d}, —R₂ₐNR_{2b}C(O)R_{2c}R_{2d}, —R₂ₐNR_{2b}C(O)OR_{2c}R_{2d}, —R₂ₐSO₂R_{2b}R_{2c}, —R₂ₐNR_{2b}SO₂NR_{2c}R_{2d} and —R₂ₐSO₂NR_{2b}R_{2c}, optionally substituted by at least one substituent independently selected from a group consisting of alkyl, cycloalkyl, heterocyclic alkyl, heteroaryl, and aryl,
wherein R₂ₐ, R_{2b}, R_{2c} and R_{2d} are independently selected from nil, H, halogen, alkyl, cycloalkyl, heterocyclic alkyl, heteroaryl, aryl or C₁-C₆ hydrocarbons, optionally substituted by at least one substituent independently selected from a group consisting of —OR₂ₑ, ═O, ═S, —SO₂R₂ₑ, —SO₂NR₂ₑR_{2f}, —NR_{2g}SO₂NR₂ₑR_{2f}, —NR_{2g}C(O)NR₂ₑR_{2f}, —C(O)NHR₂ₑ, —NHC(O)R₂ₑ, —NHC(O)OR₂ₑ, —NO₂, —CO₂R₂ₑ and —C(O)R₂ₑ,
wherein R₂ₑ, R_{2f}, and R_{2g} independently are H or alkyl.

2. The benzene fused heterocyclic compound of claim 1, wherein the alkyl is C₁-C₁₀ alkyl.

3. The benzene fused heterocyclic compound of claim 1, wherein the aryl is C₆-C₁₀ aryl.

4. The benzene fused heterocyclic compound of claim 1, having Formula (II): 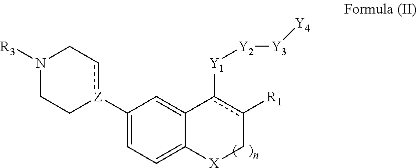
wherein [symbol] is a single or double bond;
n is 0 or 1;
X is —CH₂—, O, NR₁, or S;
Y₁ is —C(Rₐ₁)(Rₐ₂)— or —N(Rₐ₁)—, wherein Rₐ₁ and Rₐ₂ are independently selected from a group consisting of:
H, alkyl, cycloalkyl, heterocyclic alkyl, aryl, heteroaryl and C₁-C₃ hydrocarbons;
Y₂ is alkyl, cycloalkyl, heterocyclic alkyl, aryl, heteroaryl, C₁-C₃ hydrocarbon, —RₐₐOR_{bb}, —C(O)ORₐₐR_{bb}, —C(O)RₐₐR_{bb}, —C(O)NRₐₐR_{bb}, —SO₂RₐₐR_{bb} or —SO₂NRₐₐR_{bb}, wherein Rₐₐ and R_{bb} independently are nil, H, halogen, alkyl, or aryl;
Y₃ is nil, H, CN, halogen, alkyl, cycloalkyl, heterocyclic alkyl, aryl, heteroaryl or C₁-C₃ hydrocarbon, optionally substituted by at least one substituent independently selected from a group consisting of H, alkyl, and halogen;
Y₄ is nil, H, halogen, aryl or heteroaryl, optionally substituted by at least one substituent independently selected from a group consisting of H, alkyl, and halogen;
R₁ is H or alkyl;
Z is C or N;
R₃ is —R₃ₐOR_{3b}, —R₃ₐC(O)OR_{3b}R_{3c}, —R₃ₐC(O)R_{3b}R_{3c}, —R₃ₐC(O)NR_{3b}R_{3c}, —R₃ₐNR_{3b}C(O)NR_{3c}R_{3d}, —R₃ₐNR_{3b}C(O)R_{3c}R_{3d}, —R₃ₐNR_{3b}C(O)OR_{3c}R_{3d}, —R₃ₐSO₂ R_{3b}R_{3c}, —R₃ₐNR_{3b}SO₂NR_{3c}R_{3d}, or —R₃ₐSO₂NR_{3b}R_{3c}, optionally substituted by at least one substituent independently selected from a group consisting of alkyl, cycloalkyl, heterocyclic alkyl, heteroaryl, and aryl,
wherein R₃ₐ, R_{3b}, R_{3c} and R_{3d} are independently selected from a group consisting of nil, H, halogen, alkyl, cycloalkyl, heterocyclic alkyl, heteroaryl, aryl and C₁-C₆ hydrocarbon, optionally substituted by at least one substituent independently selected from a group consisting of —OR₃ₑ, ═O, ═S, —SO₂R₃ₑ, —SO₂NR₃ₑR_{3f}, —NR_{3g}SO₂NR₃ₑR_{3f}, —NR_{3g}C(O)NR₃ₑR_{3f}, —C(O)NHR₃ₑ, —NHC(O)R₃ₑ, —NHC(O)OR₃ₑ, —NO₂, —CO₂R₃ₑ and —C(O)R₃ₑ,
wherein R₃ₑ, R_{3f}, and R_{3g} independently are H or alkyl.

5. The benzene fused heterocyclic compound of claim 1, wherein the benzene fused heterocyclic compound is selected from the compounds delineated in Table A:
TABLE A

| Compound | |
|---|---|
| Number | Structure |
| 8 | 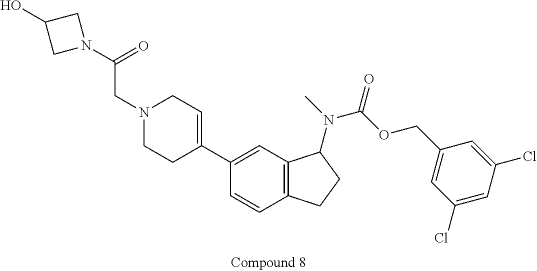 [SMILES: CN(C(=O)OCc1cc(Cl)cc(Cl)c1)C1CCc2ccc(C3=CCN(CC(=O)N4CC(O)C4)CC3)cc21] |
| 9 | 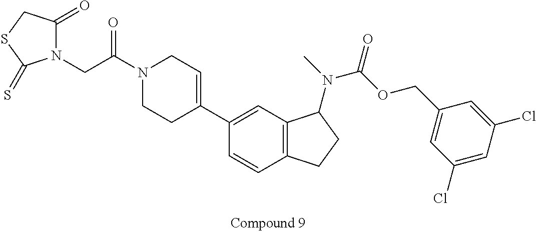 [SMILES: CN(C(=O)OCc1cc(Cl)cc(Cl)c1)C1CCc2ccc(C3=CCN(C(=O)CN4C(=O)CSC4=S)CC3)cc21] |
| 10 | 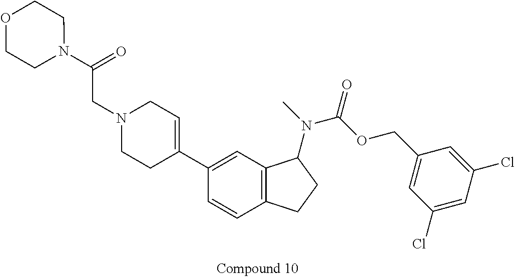 [SMILES: CN(C(=O)OCc1cc(Cl)cc(Cl)c1)C1CCc2ccc(C3=CCN(CC(=O)N4CCOCC4)CC3)cc21] |
| 11 | 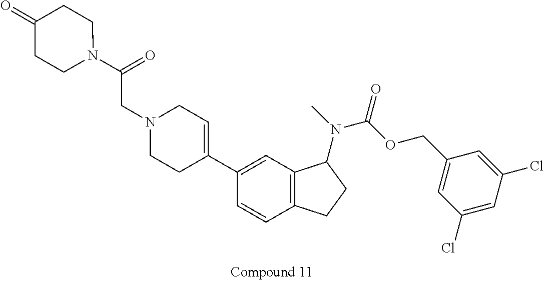 [SMILES: CN(C(=O)OCc1cc(Cl)cc(Cl)c1)C1CCc2ccc(C3=CCN(CC(=O)N4CCC(=O)CC4)CC3)cc21] |
| 12 | 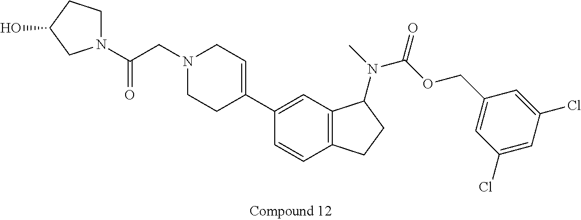 [SMILES: CN(C(=O)OCc1cc(Cl)cc(Cl)c1)C1CCc2ccc(C3=CCN(CC(=O)N4CC[C@@H](O)C4)CC3)cc21] |
| 13 | 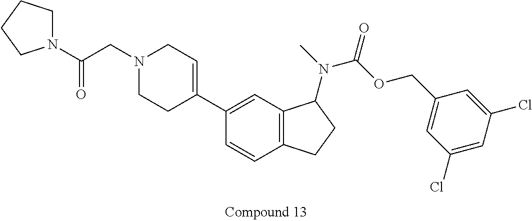 [SMILES: CN(C(=O)OCc1cc(Cl)cc(Cl)c1)C1CCc2ccc(C3=CCN(CC(=O)N4CCCC4)CC3)cc21] |
| 14 | 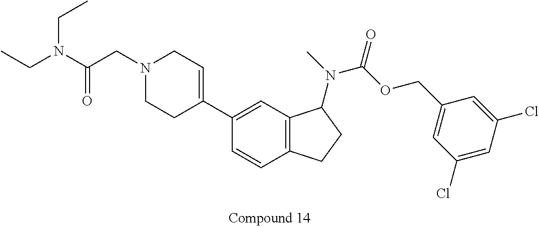 [SMILES: CCN(CC)C(=O)CN1CC=C(c2ccc3c(c2)C(N(C)C(=O)OCc2cc(Cl)cc(Cl)c2)CC3)CC1] |
| 15 | 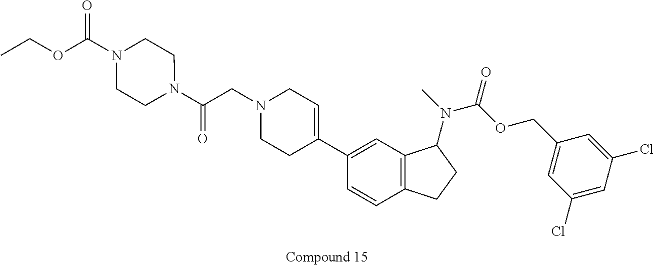 [SMILES: CCOC(=O)N1CCN(C(=O)CN2CC=C(c3ccc4c(c3)C(N(C)C(=O)OCc3cc(Cl)cc(Cl)c3)CC4)CC2)CC1] |
| 16 | 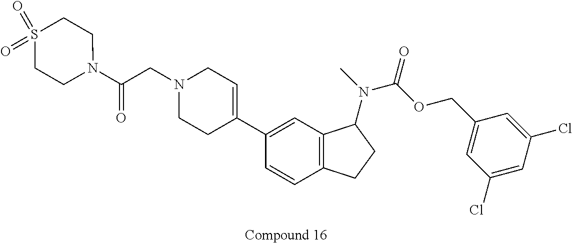 [SMILES: CN(C(=O)OCc1cc(Cl)cc(Cl)c1)C1CCc2ccc(C3=CCN(CC(=O)N4CCS(=O)(=O)CC4)CC3)cc21] |
| 17 | 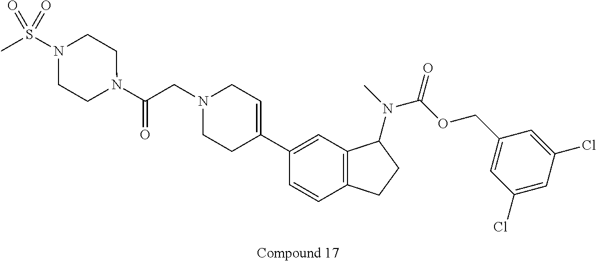 [SMILES: CN(C(=O)OCc1cc(Cl)cc(Cl)c1)C1CCc2ccc(C3=CCN(CC(=O)N4CCN(S(C)(=O)=O)CC4)CC3)cc21] |
| 18 | 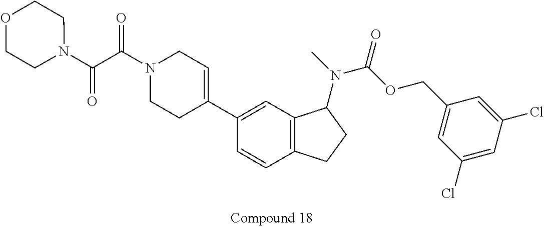 [SMILES: CN(C(=O)OCc1cc(Cl)cc(Cl)c1)C1CCc2ccc(C3=CCN(C(=O)C(=O)N4CCOCC4)CC3)cc21] |
| 19 | 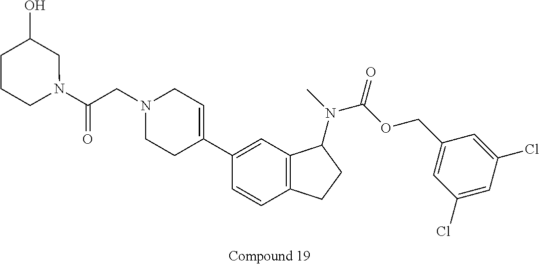 [SMILES: CN(C(=O)OCc1cc(Cl)cc(Cl)c1)C1CCc2ccc(C3=CCN(CC(=O)N4CCCC(O)C4)CC3)cc21] |
| 20 | 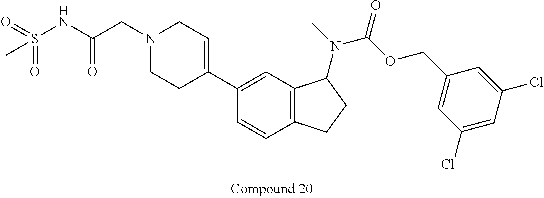 [SMILES: CN(C(=O)OCc1cc(Cl)cc(Cl)c1)C1CCc2ccc(C3=CCN(CC(=O)NS(C)(=O)=O)CC3)cc21] |
| 21 | 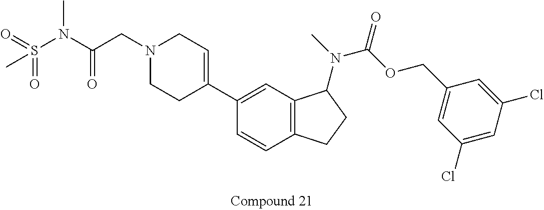 [SMILES: CN(C(=O)OCc1cc(Cl)cc(Cl)c1)C1CCc2ccc(C3=CCN(CC(=O)N(C)S(C)(=O)=O)CC3)cc21] |
| 22 | 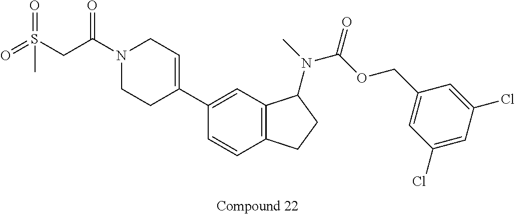 [SMILES: CN(C(=O)OCc1cc(Cl)cc(Cl)c1)C1CCc2ccc(C3=CCN(C(=O)CS(C)(=O)=O)CC3)cc21] |
| 23 | 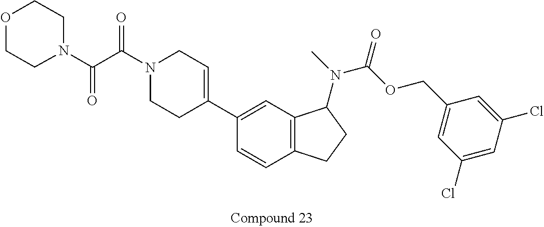 [SMILES: CN(C(=O)OCc1cc(Cl)cc(Cl)c1)C1CCc2ccc(C3=CCN(C(=O)C(=O)N4CCOCC4)CC3)cc21] |
| 24 | 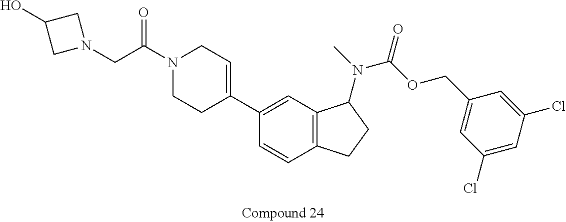 [SMILES: CN(C(=O)OCc1cc(Cl)cc(Cl)c1)C1CCc2ccc(C3=CCN(C(=O)CN4CC(O)C4)CC3)cc21] |
| 25 | 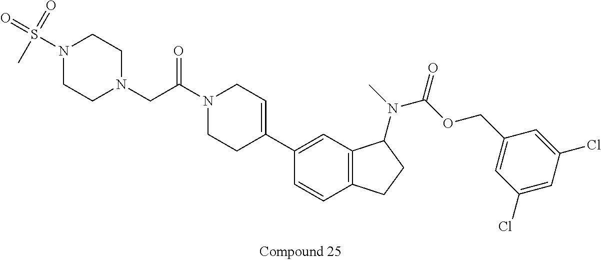 [SMILES: CN(C(=O)OCc1cc(Cl)cc(Cl)c1)C1CCc2ccc(C3=CCN(C(=O)CN4CCN(S(C)(=O)=O)CC4)CC3)cc21] |
| 26 | 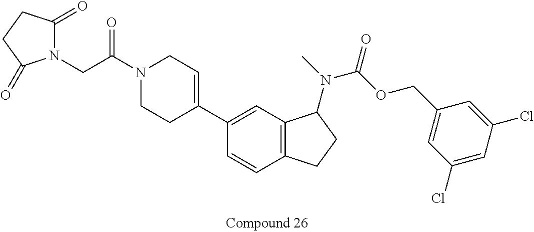 [SMILES: CN(C(=O)OCc1cc(Cl)cc(Cl)c1)C1CCc2ccc(C3=CCN(C(=O)CN4C(=O)CCC4=O)CC3)cc21] |
| 27 | 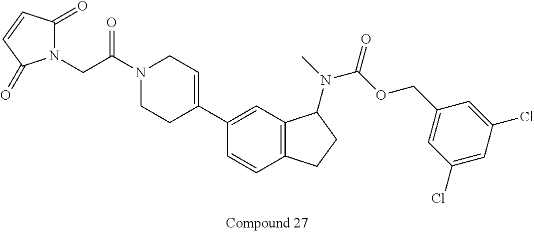 [SMILES: CN(C(=O)OCc1cc(Cl)cc(Cl)c1)C1CCc2ccc(C3=CCN(C(=O)CN4C(=O)C=CC4=O)CC3)cc21] |
| 28 | 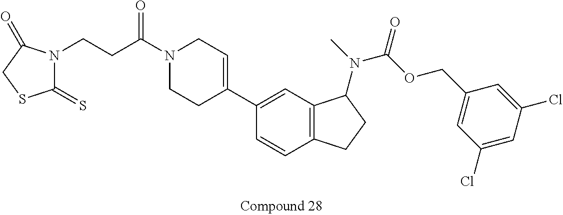 [SMILES: CN(C(=O)OCc1cc(Cl)cc(Cl)c1)C1CCc2ccc(C3=CCN(C(=O)CCN4C(=O)CSC4=S)CC3)cc21] |
| 29 | 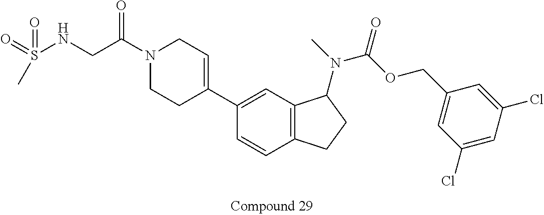 [SMILES: CN(C(=O)OCc1cc(Cl)cc(Cl)c1)C1CCc2ccc(C3=CCN(C(=O)CNS(C)(=O)=O)CC3)cc21] |
| 30 | 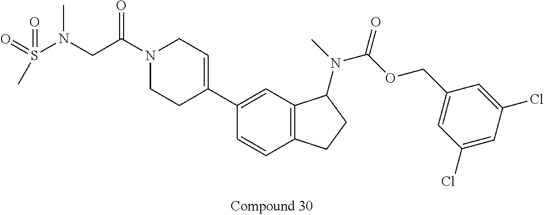 [SMILES: CN(C(=O)OCc1cc(Cl)cc(Cl)c1)C1CCc2ccc(C3=CCN(C(=O)CN(C)S(C)(=O)=O)CC3)cc21] |
| 33 | 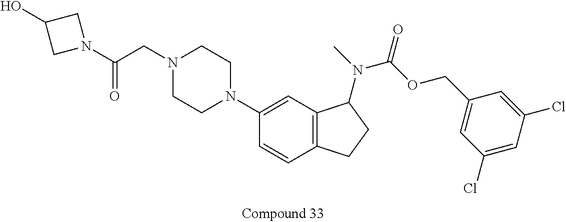 [SMILES: CN(C(=O)OCc1cc(Cl)cc(Cl)c1)C1CCc2ccc(N3CCN(CC(=O)N4CC(O)C4)CC3)cc21] |
| 34 | 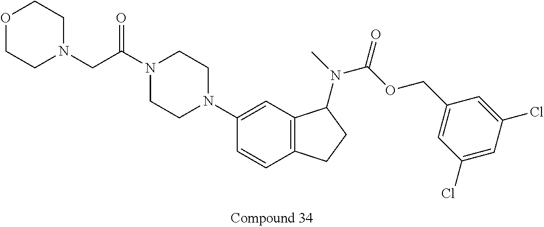 [SMILES: CN(C(=O)OCc1cc(Cl)cc(Cl)c1)C1CCc2ccc(N3CCN(C(=O)CN4CCOCC4)CC3)cc21] |
| 36 | 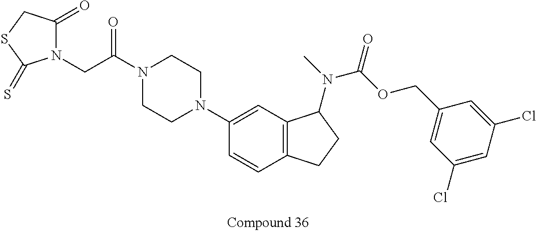 [SMILES: CN(C(=O)OCc1cc(Cl)cc(Cl)c1)C1CCc2ccc(N3CCN(C(=O)CN4C(=O)CSC4=S)CC3)cc21] |
| 37 | 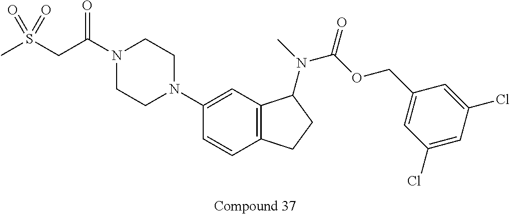 [SMILES: CN(C(=O)OCc1cc(Cl)cc(Cl)c1)C1CCc2ccc(N3CCN(C(=O)CS(C)(=O)=O)CC3)cc21] |
| 38 | 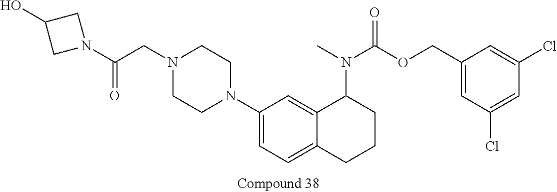 [SMILES: CN(C(=O)OCc1cc(Cl)cc(Cl)c1)C1CCCc2ccc(N3CCN(CC(=O)N4CC(O)C4)CC3)cc21] |
| 43 | 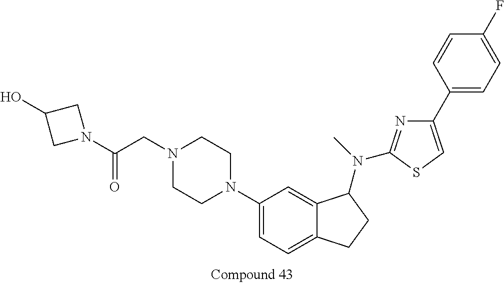 [SMILES: CN(c1nc(-c2ccc(F)cc2)cs1)C1CCc2ccc(N3CCN(CC(=O)N4CC(O)C4)CC3)cc21] |
| 44 | 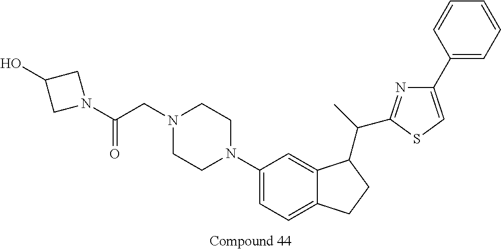 [SMILES: CC(c1nc(-c2ccccc2)cs1)C1CCc2ccc(N3CCN(CC(=O)N4CC(O)C4)CC3)cc21] |
| 50 | 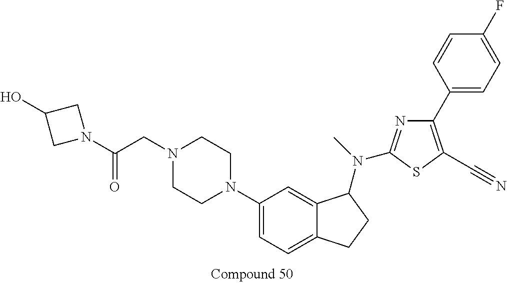 [SMILES: CN(c1nc(-c2ccc(F)cc2)c(C#N)s1)C1CCc2ccc(N3CCN(CC(=O)N4CC(O)C4)CC3)cc21] |
| 51 | 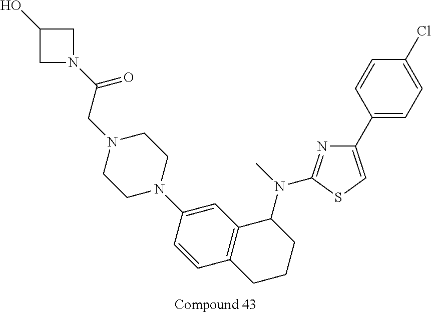 [SMILES: CN(c1nc(-c2ccc(Cl)cc2)cs1)C1CCCc2ccc(N3CCN(CC(=O)N4CC(O)C4)CC3)cc21] |
| 52 | 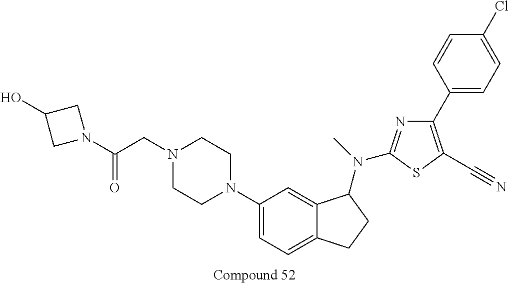 [SMILES: CN(c1nc(-c2ccc(Cl)cc2)c(C#N)s1)C1CCc2ccc(N3CCN(CC(=O)N4CC(O)C4)CC3)cc21] |
| 53 | 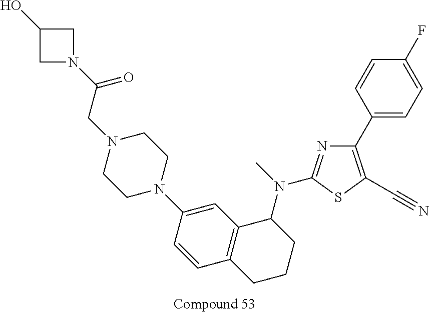 [SMILES: CN(c1nc(-c2ccc(F)cc2)c(C#N)s1)C1CCCc2ccc(N3CCN(CC(=O)N4CC(O)C4)CC3)cc21] |
| 54 | 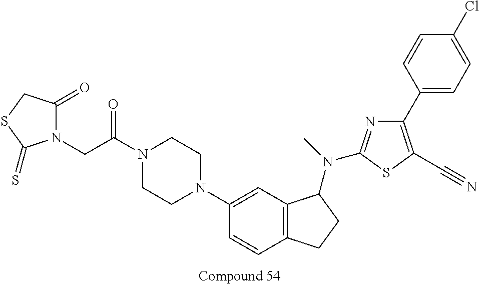 [SMILES: CN(c1nc(-c2ccc(Cl)cc2)c(C#N)s1)C1CCc2ccc(N3CCN(C(=O)CN4C(=O)CSC4=S)CC3)cc21] |
| 58 | 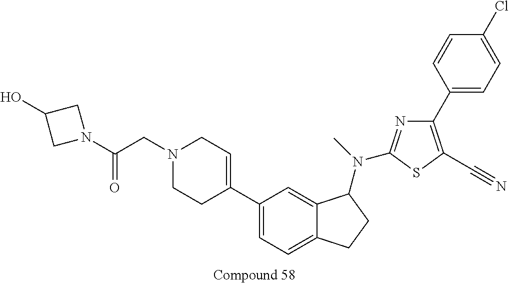 [SMILES: CN(c1nc(-c2ccc(Cl)cc2)c(C#N)s1)C1CCc2ccc(C3=CCN(CC(=O)N4CC(O)C4)CC3)cc21] |
| 59 | 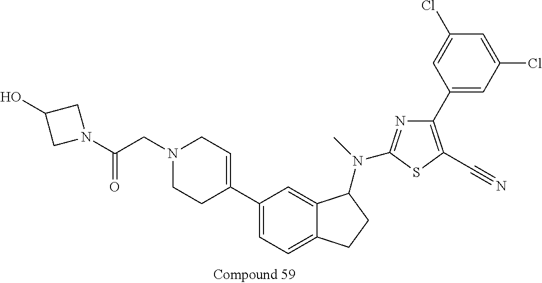 [SMILES: CN(c1nc(-c2cc(Cl)cc(Cl)c2)c(C#N)s1)C1CCc2ccc(C3=CCN(CC(=O)N4CC(O)C4)CC3)cc21] |
| 60 | 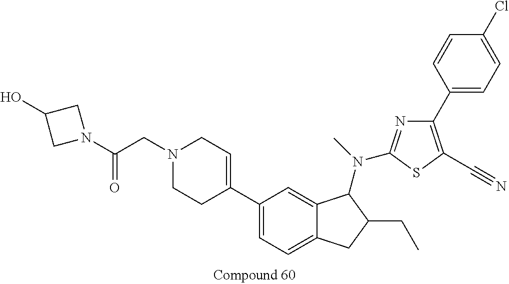 [SMILES: CCC1Cc2ccc(C3=CCN(CC(=O)N4CC(O)C4)CC3)cc2C1N(C)c1nc(-c2ccc(Cl)cc2)c(C#N)s1] |
| 61 | 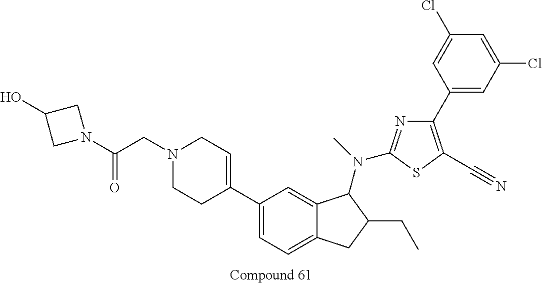 [SMILES: CCC1Cc2ccc(C3=CCN(CC(=O)N4CC(O)C4)CC3)cc2C1N(C)c1nc(-c2cc(Cl)cc(Cl)c2)c(C#N)s1] |
| 66 | 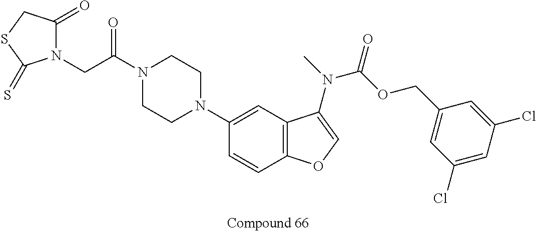 [SMILES: CN(C(=O)OCc1cc(Cl)cc(Cl)c1)c1coc2ccc(N3CCN(C(=O)CN4C(=O)CSC4=S)CC3)cc12] |
| 69 | 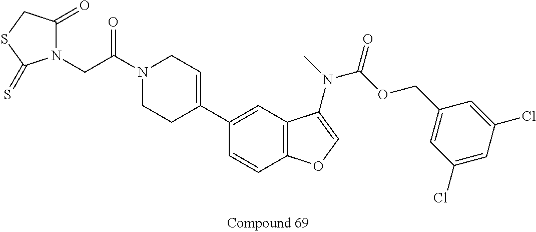 [SMILES: CN(C(=O)OCc1cc(Cl)cc(Cl)c1)c1coc2ccc(C3=CCN(C(=O)CN4C(=O)CSC4=S)CC3)cc12] |
| 73 | 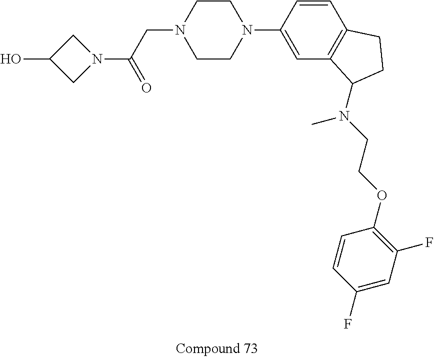 [SMILES: CN(CCOc1ccc(F)cc1F)C1CCc2ccc(N3CCN(CC(=O)N4CC(O)C4)CC3)cc21] |
| 74 | 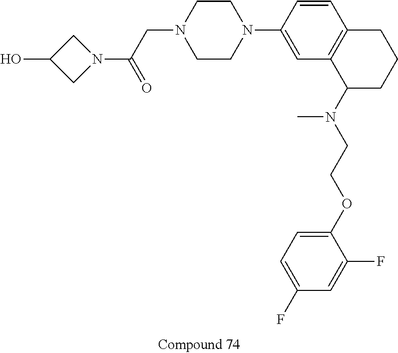 [SMILES: CN(CCOc1ccc(F)cc1F)C1CCCc2ccc(N3CCN(CC(=O)N4CC(O)C4)CC3)cc21] |
| 82 | 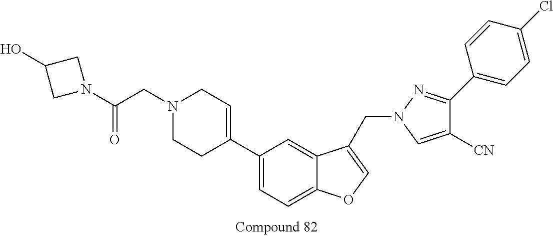 [SMILES: N#Cc1cn(Cc2coc3ccc(C4=CCN(CC(=O)N5CC(O)C5)CC4)cc23)nc1-c1ccc(Cl)cc1] |
| 85 | 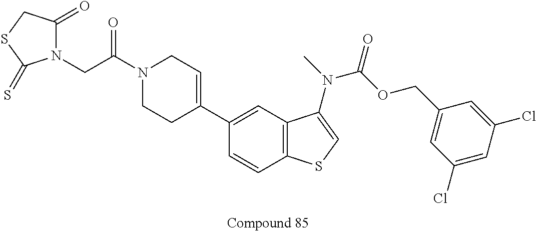 [SMILES: CN(C(=O)OCc1cc(Cl)cc(Cl)c1)c1csc2ccc(C3=CCN(C(=O)CN4C(=O)CSC4=S)CC3)cc12] |
| 92 | 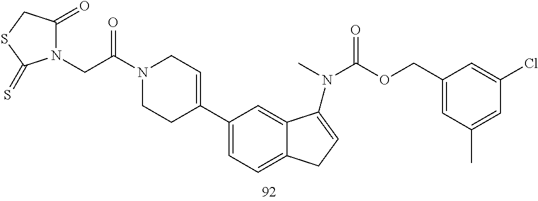 [SMILES: Cc1cc(Cl)cc(COC(=O)N(C)C2=CCc3ccc(C4=CCN(C(=O)CN5C(=O)CSC5=S)CC4)cc32)c1] |
| 93 | 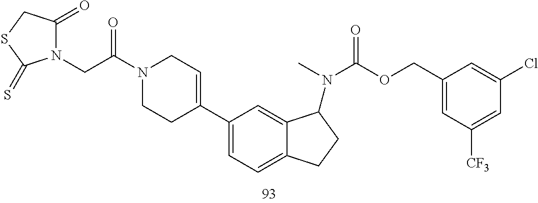 [SMILES: CN(C(=O)OCc1cc(Cl)cc(C(F)(F)F)c1)C1CCc2ccc(C3=CCN(C(=O)CN4C(=O)CSC4=S)CC3)cc21] |
| 94 | 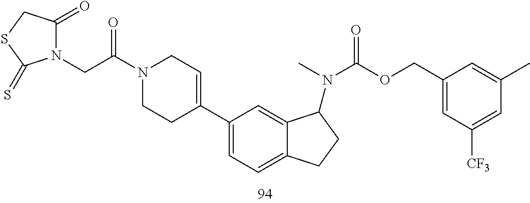 [SMILES: Cc1cc(COC(=O)N(C)C2CCc3ccc(C4=CCN(C(=O)CN5C(=O)CSC5=S)CC4)cc32)cc(C(F)(F)F)c1] |
| 95 | 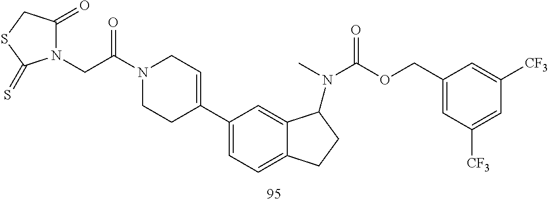 [SMILES: CN(C(=O)OCc1cc(C(F)(F)F)cc(C(F)(F)F)c1)C1CCc2ccc(C3=CCN(C(=O)CN4C(=O)CSC4=S)CC3)cc21] |
| 96 | 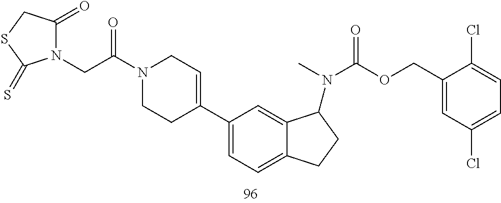 [SMILES: CN(C(=O)OCc1cc(Cl)ccc1Cl)C1CCc2ccc(C3=CCN(C(=O)CN4C(=O)CSC4=S)CC3)cc21] |
| 97 | 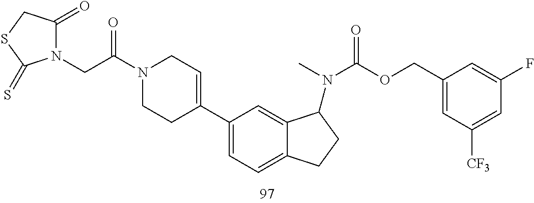 [SMILES: CN(C(=O)OCc1cc(F)cc(C(F)(F)F)c1)C1CCc2ccc(C3=CCN(C(=O)CN4C(=O)CSC4=S)CC3)cc21] |
| 102 | 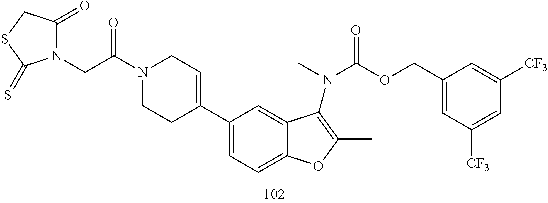 [SMILES: Cc1oc2ccc(C3=CCN(C(=O)CN4C(=O)CSC4=S)CC3)cc2c1N(C)C(=O)OCc1cc(C(F)(F)F)cc(C(F)(F)F)c1] |
| 103 | 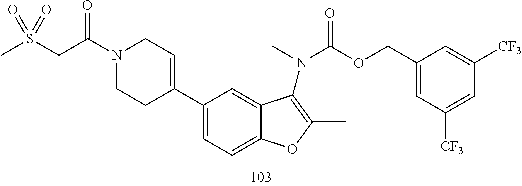 [SMILES: Cc1oc2ccc(C3=CCN(C(=O)CS(C)(=O)=O)CC3)cc2c1N(C)C(=O)OCc1cc(C(F)(F)F)cc(C(F)(F)F)c1] |
| 105 | 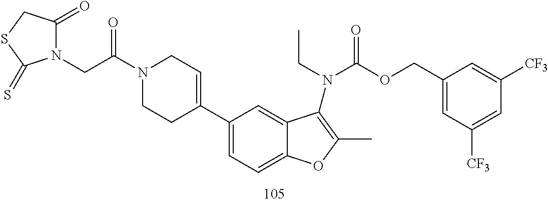 [SMILES: CCN(C(=O)OCc1cc(C(F)(F)F)cc(C(F)(F)F)c1)c1c(C)oc2ccc(C3=CCN(C(=O)CN4C(=O)CSC4=S)CC3)cc12] |
| 109 | 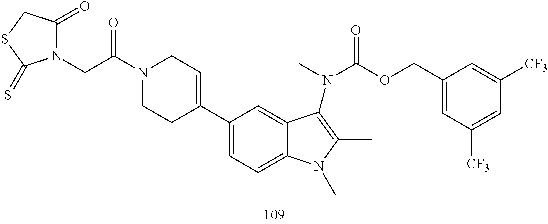 [SMILES: Cc1c(N(C)C(=O)OCc2cc(C(F)(F)F)cc(C(F)(F)F)c2)c2cc(C3=CCN(C(=O)CN4C(=O)CSC4=S)CC3)ccc2n1C] |
| 112 | 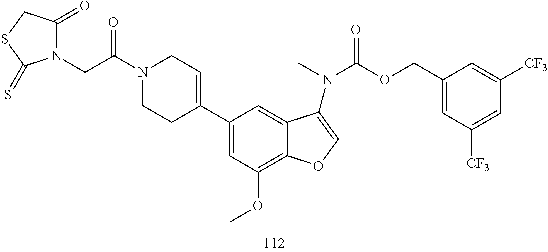 [SMILES: COc1cc(C2=CCN(C(=O)CN3C(=O)CSC3=S)CC2)cc2c(N(C)C(=O)OCc3cc(C(F)(F)F)cc(C(F)(F)F)c3)coc12] |
| 113 | 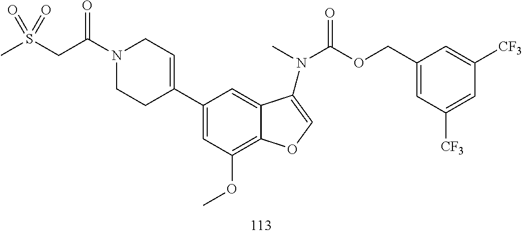 [SMILES: COc1cc(C2=CCN(C(=O)CS(C)(=O)=O)CC2)cc2c(N(C)C(=O)OCc3cc(C(F)(F)F)cc(C(F)(F)F)c3)coc12] |
| 117 | 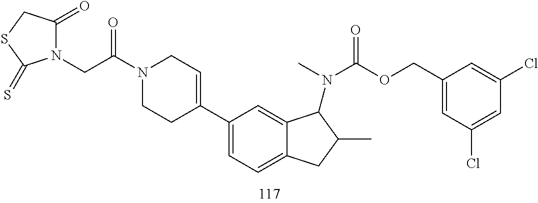 [SMILES: CC1Cc2ccc(C3=CCN(C(=O)CN4C(=O)CSC4=S)CC3)cc2C1N(C)C(=O)OCc1cc(Cl)cc(Cl)c1] |
| 119 | 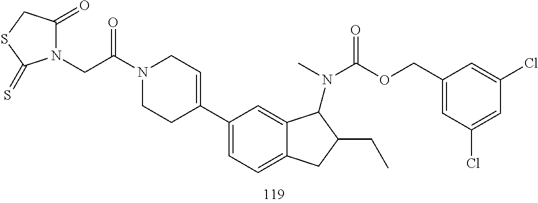 [SMILES: CCC1Cc2ccc(C3=CCN(C(=O)CN4C(=O)CSC4=S)CC3)cc2C1N(C)C(=O)OCc1cc(Cl)cc(Cl)c1] |
| 120 | 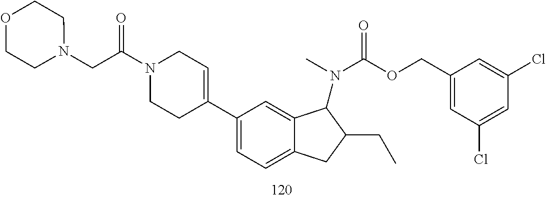 [SMILES: CCC1Cc2ccc(C3=CCN(C(=O)CN4CCOCC4)CC3)cc2C1N(C)C(=O)OCc1cc(Cl)cc(Cl)c1] |
| 121 | 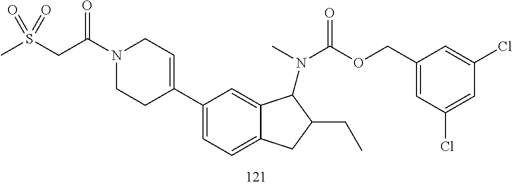 [SMILES: CCC1Cc2ccc(C3=CCN(C(=O)CS(C)(=O)=O)CC3)cc2C1N(C)C(=O)OCc1cc(Cl)cc(Cl)c1] |
| 127 | 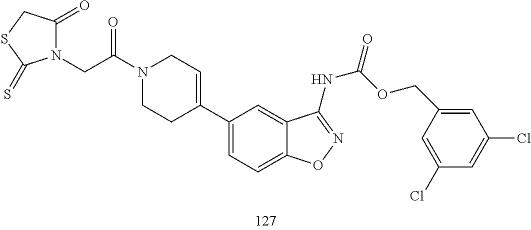 [SMILES: O=C(Nc1noc2ccc(C3=CCN(C(=O)CN4C(=O)CSC4=S)CC3)cc12)OCc1cc(Cl)cc(Cl)c1] |
| 128 | 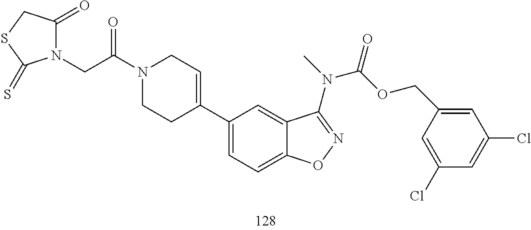 [SMILES: CN(C(=O)OCc1cc(Cl)cc(Cl)c1)c1noc2ccc(C3=CCN(C(=O)CN4C(=O)CSC4=S)CC3)cc12] |
| 129 | 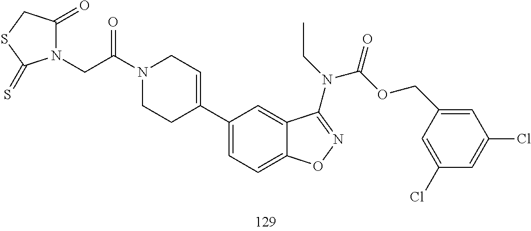 [SMILES: CCN(C(=O)OCc1cc(Cl)cc(Cl)c1)c1noc2ccc(C3=CCN(C(=O)CN4C(=O)CSC4=S)CC3)cc12] |
| 130 | 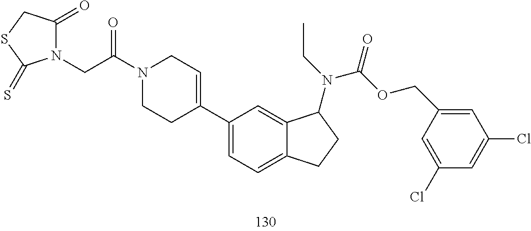 [SMILES: CCN(C(=O)OCc1cc(Cl)cc(Cl)c1)C1CCc2ccc(C3=CCN(C(=O)CN4C(=O)CSC4=S)CC3)cc21] |
| 131 | 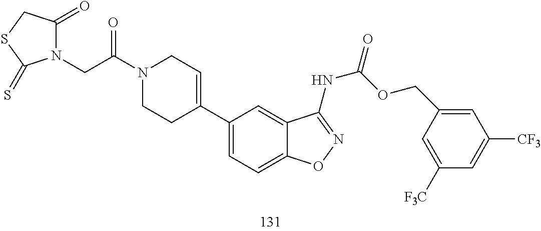 [SMILES: O=C(Nc1noc2ccc(C3=CCN(C(=O)CN4C(=O)CSC4=S)CC3)cc12)OCc1cc(C(F)(F)F)cc(C(F)(F)F)c1] |
| 132 | 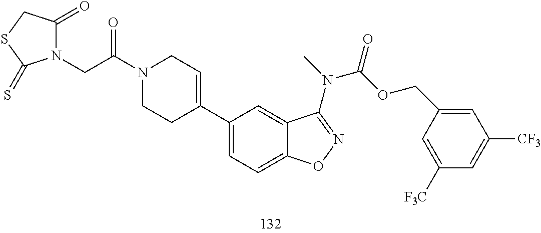 [SMILES: CN(C(=O)OCc1cc(C(F)(F)F)cc(C(F)(F)F)c1)c1noc2ccc(C3=CCN(C(=O)CN4C(=O)CSC4=S)CC3)cc12] |
| 133 | 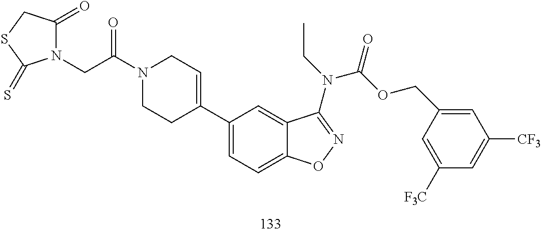 [SMILES: CCN(C(=O)OCc1cc(C(F)(F)F)cc(C(F)(F)F)c1)c1noc2ccc(C3=CCN(C(=O)CN4C(=O)CSC4=S)CC3)cc12] |
| 137 | 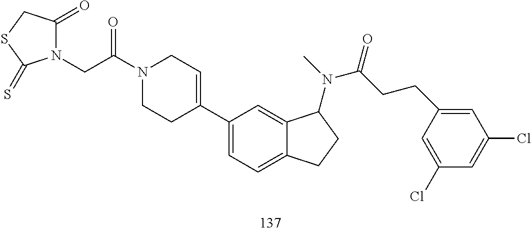 [SMILES: CN(C(=O)CCc1cc(Cl)cc(Cl)c1)C1CCc2ccc(C3=CCN(C(=O)CN4C(=O)CSC4=S)CC3)cc21] |
| 138 | 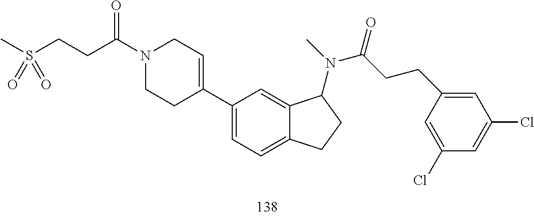 [SMILES: CN(C(=O)CCc1cc(Cl)cc(Cl)c1)C1CCc2ccc(C3=CCN(C(=O)CCS(C)(=O)=O)CC3)cc21] |
| 139 | 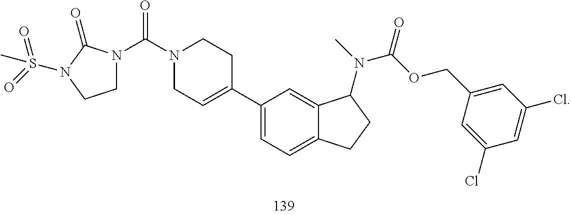 [SMILES: CN(C(=O)OCc1cc(Cl)cc(Cl)c1)C1CCc2ccc(C3=CCN(C(=O)N4CCN(S(C)(=O)=O)C4=O)CC3)cc21] |

6. A pharmaceutical composition, comprising:
a therapeutically effective amount of the benzene fused heterocyclic compound of claim 1; and
a pharmaceutically acceptable carrier.

7. The pharmaceutical composition of claim 6; wherein the pharmaceutically acceptable carrier is selected from the group consisting of inert diluents, dispersing and/or granulating agents, surface active agents and/or emulsifiers, disintegrating agents, binding agents, preservatives, buffering agents, lubricating agents, and oils.

8. A method for inhibiting activity of autotaxin in environment, comprising:
contacting the environment with an effective amount of the benzene fused heterocyclic compound of claim 1.

9. The method of claim 8, wherein the environment is a cell.

10. A method for inhibiting activity of autotaxin in environment, comprising:
contacting the environment with an effective amount of the pharmaceutical composition of claim 6.

11. The method of claim 10, wherein the environment is a cell.
